(19) United States
(12) Patent Application Publication
(10) Pub. No.: US 2026/0226035 A1
(43) Pub. Date: Aug. 6, 2026

(54) THIOCHROMAN DERIVATIVE AND PREPARATION METHOD THEREFOR AND USE THEREOF

(71) Applicant: SHANGHAI INSTITUTE OF MATERIA MEDICA, CHINESE ACADEMY OF SCIENCES, Shanghai (CN)

(72) Inventors: Yushe YANG, Shanghai (CN); Dan ZHANG, Shanghai (CN); Zhengyu LU, Shanghai (CN)

(21) Appl. No.: 19/120,940

(22) PCT Filed: Oct. 10, 2023
(86) PCT No.: PCT/CN2023/123758

§ 371 Date: Apr. 14, 2025

(30) Foreign Application Priority Data
Oct. 14, 2022 (CN) 202211262160.4

(51) Int. Cl. C07D 409/12; A61K 31/397; A61K 31/4155; A61K 31/4162; A61K 31/4433; A61K 31/453; A61K 31/69; A61P 35/00; C07D 405/14; C07D 495/04; C07F 5/02

(52) U.S. Cl. CPC C07D 409/12; A61K 31/397; A61K 31/4155; A61K 31/4162; A61K 31/4433; A61K 31/453; A61K 31/69; A61P 35/00; C07D 405/14; C07D 495/04; C07F 5/025

(57) ABSTRACT

A thiochroman derivative and a preparation method therefor and a use thereof are provided. The structure of the thiochroman derivative is as shown in general formula (I), and the definition of each substituent is as described in the description and the claims. The thiochroman derivative can be used as a selective estrogen receptor degrader and antagonist for treating estrogen receptor positive diseases.

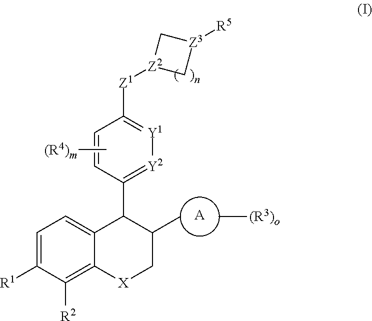

### TECHNICAL FIELD

The present invention belongs to the field of medicine, and relates to a class of thiochroman compounds, the preparation method and medical application thereof. In particular, the present invention relates to thiochroman derivatives represented by general formula (I), the racemate, enantiomer, diastereomer and mixtures thereof, the pharmaceutically acceptable salt thereof and a pharmaceutical composition containing the compounds, and use of the compound for treating, preventing or diagnosing estrogen receptor-sensitive, estrogen receptor-mediated or dependent disease or condition, and the diseases is particularly preferably estrogen receptor-positive breast cancer.

### BACKGROUND TECHNIQUE

Breast cancer is the world's leading cancer, with 2.26 million new cases worldwide in 2020. Although the five-year survival rate of early breast cancer exceeds 90%, it still causes nearly 700,000 deaths each year due to tumor metastasis and spread. More than 70% of breast cancer cases highly express estrogen receptors (ER). ER binds to estrogen, activates signaling pathways, and thus promotes the occurrence and development of breast cancer. It is the main driving factor for the occurrence and development of breast cancer.

Endocrine therapy is the first choice for the treatment of ER-positive breast cancer. Standard treatment drugs include: (1) aromatase/estrogen synthase inhibitors, whose main mechanism is to inhibit the biosynthesis of endogenous estrogen. Representative drugs include letrozole and anastrozole. The main side effects of this type of drug are accelerated bone density loss and widespread acquired drug resistance; (2) selective estrogen receptor modulators (SERM) that antagonize ER activity, namely tamoxifen. The disadvantage is that long-term use can lead to hormone-independent drug resistance, and the drug has a partial agonist effect, which increases the risk of endometrial hyperplasia, polyp and endometrial cancer. Although endocrine therapy has made great progress, 30-50% of patients will develop drug resistance within 5 years of endocrine therapy and then develop disease progression and metastasis. The 5-year survival rate is only about 20%, and the median overall survival time is only 2 to 3 years. Acquired drug resistance dominated by ESR1-LBD and distant metastasis represented by bone, brain, liver, lung and lymph node metastasis are important reasons for the increasing number of patients with treatment resistance and drug resistance, especially brain metastasis (10-15% of patients with brain metastasis). There is currently no ideal treatment drug and treatment method, and the prognosis of patients is very poor. The median survival time after clinical drug treatment is only 2-9 months. In these resistant breast cancers, ER signaling is still a key driver. Therefore, using new methods to target ER and block its signaling pathway remains the key to develop new drugs for the treatment of breast cancer.

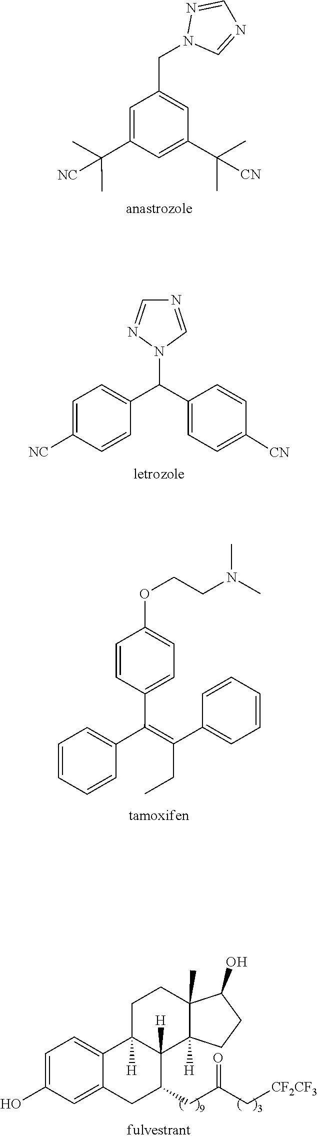

Selective Estrogen Receptor Degraders (SERD) are a novel therapeutic option. SERDs inhibit tumor cell proliferation by completely blocking ER signaling through binding to ER, resulting in the inability of ER to activate the transcription of target genes and prompting ER ubiquitination degradation, and this effect is not affected by the mutation status of common mutation ESR1, so it can overcome acquired resistance and side effects produced by endocrine therapy, and is expected to become the pillar therapy for ER-positive breast cancer. Fulvestrant is the only SERD drug currently on the market. It is used to treat patients with ER+ locally advanced or metastatic postmenopausal breast cancer who have relapsed or progressed after endocrine therapy, or postmenopausal patients with HR⁺ (progesterone receptor positive)/HER2⁻ (human epidermal growth factor receptor-2 negative) locally advanced/metastatic breast cancer who have not received endocrine therapy before. The excellent anti-tumor activity of Fulvestrant (superior to estrogen receptor modulators) has been widely recognized clinically, but due to the characteristics of its steroidal structure, it has poor solubility, slow absorption, low in vivo exposure, extremely low bioavailability, and metabolic instability. As a result, it cannot be administered orally and can only be injected intramuscularly. Intramuscular injection (a single intramuscular injection can only reach a maximum of 500 mg) is difficult to achieve the in vivo drug concentration required for optimal efficacy. It takes 3-6 months after administration to achieve a stable plasma concentration, and severe pain, swelling, redness and other reactions at the injection site also make the patient's compliance very poor, greatly limiting its clinical application. In addition, Fulvestrant cannot pass through the blood-brain barrier and cannot be used to treat brain metastases, which is extremely difficult in the treatment of breast cancer. Therefore, the development of oral small molecule SERDs with good oral absorption, oral effectiveness, more ideal pharmacokinetic properties, and the ability to pass through the blood-brain barrier is a huge unmet clinical need.

Currently, there are multiple candidate SERDs drug molecules in different stages of clinical research, and SERDs/antagonists with public structures include LSZ-102 (U.S. Pat. No. 9,322,746), ZB716 (WO2016004166), RAD1901 (WO2016176666), SAR439859 (WO2018091153), AZD9833 (WO2019002442), and GDC-9545 (WO2016097072). So far, no orally effective small molecule SERDs have been approved for marketing.

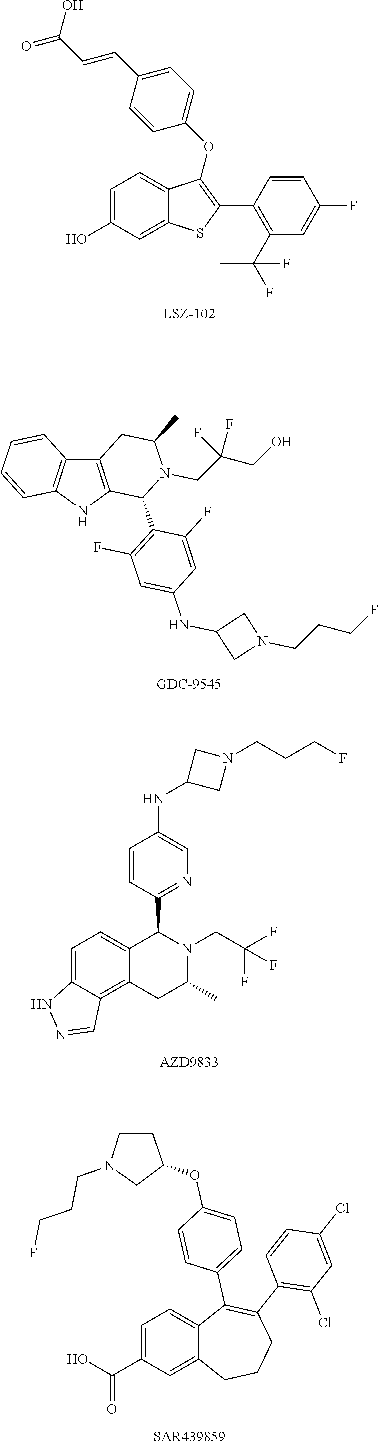

Currently, the treatment options for patients with estrogen-positive breast cancer that is resistant to endocrine therapy are very limited. The only available drug, fulvestrant, has many problems, especially for patients having breast cancer with brain metastases, which is almost helpless.

### SUMMARY OF THE INVENTION

The object of the present invention is to provide a SERD with novel structure, excellent ER antagonism and degradation activity, oral efficacy and good brain distribution.

The first aspect of the present invention provides a compound represented by general formula (I), or a racemate, enantiomer, diastereomer, or a mixture thereof, or a pharmaceutically acceptable salt thereof:

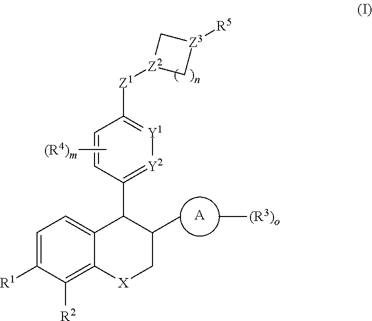

wherein: R¹ is selected from OH, COOH, B(OH)₂, halogen, C₁-C₆ alkyl, halogenated C₁-C₆ alkyl or C₁-C₆ alkoxy; R² is selected from H, OH, COOH, halogen, cyano, C₁-C₆ alkyl, C₁-C₆ alkoxy, halogenated C₁-C₆ alkyl or hydroxy-substituted C₁-C₆ alkyl; or R¹, R² together with connected benzene ring form a benzo 5-6 membered heteroaryl;
X is selected from S, S(O)₂ or O;
ring A is selected from C₃-C₆ cycloalkyl, 5-8 membered heterocyclyl, C₆-C₁₀ aryl or 5-8 membered heteroaryl;
each R³ is independently selected from hydrogen, halogen, cyano, C₁-C₆ alkylthio, C₁-C₆ alkylCO—, C₁-C₆ alkylSO₂—, amino, —NH(C₁-C₆ alkyl), —N(C₁-C₆ alkyl)(C₁-C₆ alkyl), —SO₂NH₂, —C(O)NH₂, C₁-C₆ alkyl, C₁-C₆ alkoxy, halogenated C₁-C₆ alkyl, halogenated C₁-C₆ alkoxy or halogenated C₁-C₆ alkylthio; o is 0, 1, 2, 3 or 4;
Y¹, Y² are independently selected from CR⁴ or N;
each R⁴ is independently selected from hydrogen, halogen, cyano, C₁-C₆ alkylthio, C₁-C₆ alkylCO—, C₁-C₆ alkylSO₂—, amino, —NH(C₁-C₆ alkyl), —N(C₁-C₆ alkyl)(C₁-C₆ alkyl), —SO₂NH₂, —C(O)NH₂, C₁-C₆ alkyl, C₁-C₆ alkoxy, halogenated C₁-C₆ alkyl, halogenated C₁-C₆ alkoxy or halogenated C₁-C₆ alkylthio;
m is 0, 1, 2, 3 or 4;
Z¹-Z² is selected from O—Z², NH—Z², S—Z², S(O)—Z², S(O)₂—Z², O—(C₁-C₆ alkylene)-Z², O-(halogenated C₁-C₆ alkylene)-Z², NH—(C₁-C₆ alkylene)-Z² or NH-(halogenated C₁-C₆ alkylene)-Z²;
Z², Z³ are independently selected from CH or N; n is 1, 2 or 3;
R⁵ is C₁-C₆ alkyl, optionally substituted by one or more substituents selected from the group consisting of halogen, cyano, hydroxyl, carboxyl, amino, methoxy or —SO₂CH₃;
the premise is that when X is S or S(O)₂, ring A is selected from C₃-C₆ cycloalkyl, 5-8 membered heterocyclyl, C₆-C₁₀ aryl or 5-8 membered heteroaryl; when X is O, ring A is selected from C₃-C₆ cycloalkyl, 5-8 membered heterocyclyl or 5-8 membered heteroaryl.

In another preferred embodiment, each R³ is independently selected from hydrogen, halogen, cyano, C₁-C₄ alkylthio, C₁-C₄ alkylCO—, C₁-C₄ alkylSO₂—, amino, —NH(C₁-C₄ alkyl), —N(C₁-C₄ alkyl)(C₁-C₄ alkyl), —SO₂NH₂, —C(O)NH₂, C₁-C₄ alkyl, C₁-C₄ alkoxy, halogenated C₁-C₄ alkyl, halogenated C₁-C₄ alkoxy or halogenated C₁-C₄ alkylthio;
o is 0, 1, 2, 3, or 4;
when X is S or S(O)₂, ring A is selected from C₃-C₆ cycloalkyl, 5-7 membered heterocyclyl, C₆-C₁₀ aryl or 5-7 membered heteroaryl; when X is O, ring A is selected from C₃-C₆ cycloalkyl, 5-7 membered heterocyclyl or 5-7 membered heteroaryl.

In another preferred embodiment, Z¹-Z² is selected from O—Z², NH—Z², S—Z², S(O)—Z², S(O)₂—Z², O—(C₁-C₄ alkylene)-Z², O-(halogenated C₁-C₄ alkylene)-Z², NH—(C₁-C₄ alkylene)-Z² or NH-(halogenated C₁-C₄ alkylene)-Z²;
Z², Z³ are independently selected from CH or N;
n is 1, 2 or 3;
R⁵ is C₁-C₄ alkyl, optionally substituted by one or more substituents selected from the group consisting of fluorine, chlorine, bromine, cyano, hydroxyl or carboxyl.

In another preferred embodiment, R¹ is selected from OH, COOH, B(OH)₂, R² is selected from H; or R¹, R² together with the connected benzene ring form

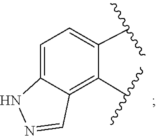 [SMILES: *c1ccc2[nH]ncc2c1*]

X is selected from S or S(O)₂;
ring A is selected from C₃-C₆ cycloalkyl, 5-6 membered heterocyclyl, phenyl or 5-6 membered heteroaryl;
each R³ is independently selected from hydrogen, fluorine, chlorine, bromine, cyano, C₁-C₄ alkylthio, C₁-C₂ alkylCO—, C₁-C₂ alkylSO₂—, amino, —NH(C₁-C₂ alkyl), —N(C₁-C₂ alkyl)(C₁-C₂ alkyl), —SO₂NH₂, —C(O)NH₂, C₁-C₄ alkyl, C₁-C₄ alkoxy, halogenated C₁-C₄ alkyl, halogenated C₁-C₄ alkoxy or halogenated C₁-C₄ alkylthio;
o is 0, 1, 2, 3, or 4;
Y¹, Y² are independently selected from CR⁴ or N;
each R⁴ is independently selected from hydrogen, fluorine, chlorine, bromine, cyano, C₁-C₂ alkylthio, C₁-C₂ alkylCO—, C₁-C₂ alkylSO₂—, amino, —NH(C₁-C₂ alkyl), —N(C₁-C₂ alkyl)(C₁-C₂ alkyl), —SO₂NH₂, —C(O)NH₂, C₁-C₄ alkyl, C₁-C₄ alkoxy, halogenated C₁-C₄ alkyl, halogenated C₁-C₄ alkoxy or halogenated C₁-C₄ alkylthio;
m is 0, 1, 2 or 3;
Z¹-Z² is selected from O—Z², NH—Z², O—(C₁-C₄ alkylene)-Z², O-(halogenated C₁-C₄ alkylene)-Z², NH—(C₁-C₄ alkylene)-Z² or NH-(halogenated C₁-C₄ alkylene)-Z²;
Z², Z³ are independently selected from CH or N;
n is 1 or 2;
R⁵ is C₁-C₄ alkyl, optionally substituted by one or more substituents selected from the group consisting of fluorine, chlorine, bromine, cyano, hydroxyl or carboxyl.

In another preferred embodiment, R¹ is selected from OH, COOH, B(OH)₂, R² is selected from H; or R¹, R² together with the connected benzene ring form

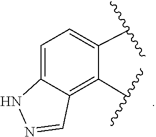 [SMILES: *c1ccc2[nH]ncc2c1*]

In another preferred embodiment, when X is S or S(O)₂, ring A is selected from C₃-C₆ cycloalkyl, 5-7 membered heterocyclyl, phenyl or 5-7 membered heteroaryl; when X is O, ring A is selected from C₃-C₆ cycloalkyl, 5-7 membered heterocyclyl or 5-7 membered heteroaryl.

In another preferred embodiment, when X is S or S(O)₂, ring A is selected from C₃-C₆ cycloalkyl, 5-7 membered heterocyclyl, C₆-C₁₀ aryl or 5-7 membered heteroaryl; in another preferred embodiment, ring A is selected from: phenyl, 5-6 membered heterocyclyl (having 1 or 2 heteroatoms selected from the following group: N, O) or 5-6 membered heteroaryl (having 1 or 2 N); in another preferred embodiment, ring A is selected from: phenyl, piperidinyl, pyridinyl, pyranyl, tetrahydropyranyl, pyrazolyl, pyrrolyl, imidazolyl, pyridazinyl, pyrimidinyl, pyrazinyl.

In another preferred embodiment, X is S or S(O)₂; and ring A is phenyl.

In another preferred embodiment, X is O; ring A is a 5-6 membered heterocyclyl (having 1 or 2 heteroatoms selected from the group consisting of N and O) or 5-6 membered heteroaryl (having 1 or 2N).

In another preferred embodiment, X is O; and ring A is piperidinyl, pyridinyl, pyrazolyl, tetrahydropyranyl, pyranyl, pyrrolyl, imidazolyl, pyridazinyl, or pyrimidinyl.

In another preferred embodiment, each R³ is independently selected from hydrogen, halogen, cyano, C₁-C₄ alkylthio, C₁-C₄ alkylCO—, C₁-C₄ alkylSO₂—, amino, —NH(C₁-C₄ alkyl), —N(C₁-C₄ alkyl)(C₁-C₄ alkyl), —SO₂NH₂, —C(O)NH₂, C₁-C₄ alkyl, C₁-C₄ alkoxy, halogenated C₁-C₄ alkyl, halogenated C₁-C₄ alkoxy or halogenated C₁-C₄ alkylthio; o is 0, 1, 2, 3 or 4.

In another preferred embodiment, R³ is selected from hydrogen, halogen, cyano, thiomethyl, acetyl, methanesulfonyl, —NMe₂, —SO₂NH₂, —C(O)NH₂, C₁-C₄ alkyl, C₁-C₄ alkoxy, halogenated C₁-C₄ alkyl, halogenated C₁-C₄ alkoxy or halogenated C₁-C₄ alkylthio.

In another preferred embodiment, each R³ is independently selected from hydrogen, fluorine, chlorine, bromine, CH₃CO—, CH₃SO₂—, CH₃CH₂CO—, CH₃CH₂SO₂—, amino, cyano, —NHCH₃, —N(CH₃)₂, —SO₂NH₂, —C(O)NH₂, methyl, ethyl, n-propyl, isopropyl, n-butyl, isobutyl, methoxy, ethoxy, propoxy, butoxy, halogenated C₁-C₂ alkyl, halogenated C₁-C₄ alkoxy or halogenated C₁-C₄ alkylthio; o is 0, 1, 2, 3 or 4.

In another preferred embodiment, Y¹ is CH, CF or N.

In another preferred embodiment, Y² is CH or N.

In another preferred embodiment, the structural unit

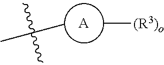

is selected from

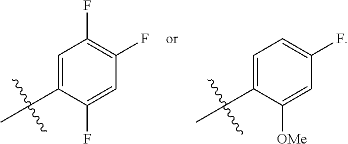 [SMILES: *c1cc(F)c(F)cc1F ; *c1ccc(F)cc1OC]

In another preferred embodiment, the halogenated C₁-C₂ alkyl is selected from —CH₂F, —CHF₂, —CF₃, —CH₂CF₃, and —CH₂CH₂F.

In another preferred embodiment, R⁴ is selected from hydrogen, halogen, cyano, thiomethyl, acetyl, methanesulfonyl, —NMe₂, —SO₂NH₂, —C(O)NH₂, C₁-C₄ alkyl, C₁-C₄ alkoxy, halogenated C₁-C₄ alkyl, halogenated C₁-C₄ alkoxy or halogenated C₁-C₄ alkylthio; m is 0, 1, 2 or 3.

In another preferred embodiment, each R⁴ is independently selected from hydrogen, fluorine, chlorine, and bromine; m is 0, 1, 2 or 3.

In another preferred embodiment, Z¹-Z² is selected from O—Z², NH—Z², S—Z², S(O)—Z², S(O)₂—Z², O—(C₁-C₄ alkylene)-Z², O-(halogenated C₁-C₄ alkylene)-Z², NH—(C₁-C₄ alkylene)-Z² or NH-(halogenated C₁-C₄ alkylene)-Z². In another preferred embodiment, the above-mentioned halogenated refers to fluoro, chloro or bromo.

In another preferred embodiment, Z¹-Z² is selected from O—Z², NH—Z², —OCH₂—Z², —OCH₂CH₂—Z², —OCH₂CH₂CH₂—Z², O-(fluoro-C₁-C₂ alkylene)-Z², NH—(C₁-C₂ alkylene)-Z² or NH-(fluoro-C₁-C₂ alkylene)-Z², O-(chloro-C₁-C₂ alkylene)-Z², NH—(C₁-C₂ alkylene)-Z² or NH-(chloro-C₁-C₂ alkylene)-Z².

In another preferred embodiment, Z² is CH, Z³ is N; Z² is N, and Z³ is CH.

In another preferred embodiment, n is 1, 2 or 3.

In another preferred embodiment, R⁵ is —CH₂F, —CHF₂, —CF₃, —CH₂CN or —CH₂CH₂CH₂F. In another preferred embodiment, R⁵ is —CH₂F.

In another preferred embodiment, the structural unit

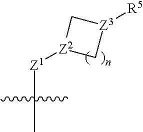

is selected from

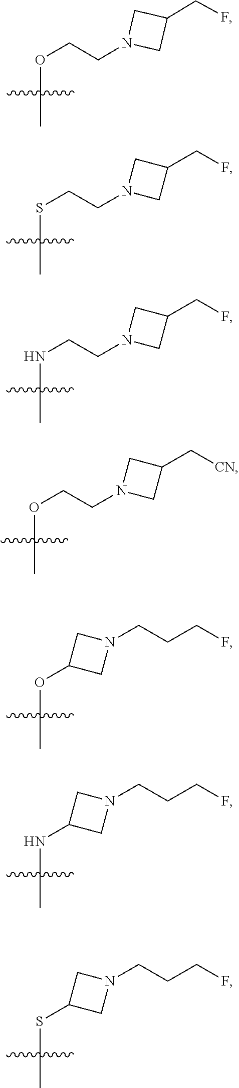

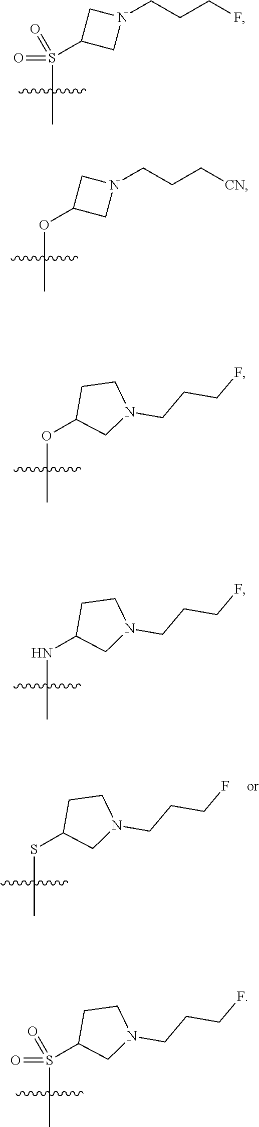 [SMILES: *S(=O)(=O)C1CN(CCCF)C1 ; *OC1CN(CCCC#N)C1 ; *OC1CCN(CCCF)C1 ; *NC1CCN(CCCF)C1 ; *SC1CCN(CCCF)C1 ; *S(=O)(=O)C1CCN(CCCF)C1]

In another preferred embodiment, the structural unit

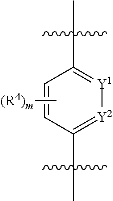

is selected from

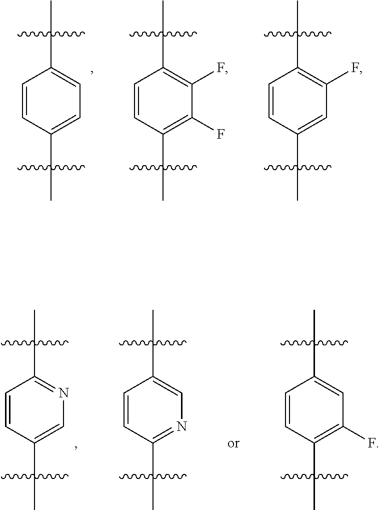 [SMILES: *c1ccc(*)cc1 ; *c1ccc(*)c(F)c1F ; *c1ccc(*)c(F)c1 ; *c1ccc(*)nc1 ; *c1ccc(*)nc1 ; *c1ccc(*)c(F)c1]

In another preferred embodiment, when X is O, ring A is selected from 5-6 membered cycloalkyl, 5-6 membered heterocycloalkyl or 5-6 membered heteroaryl; preferably

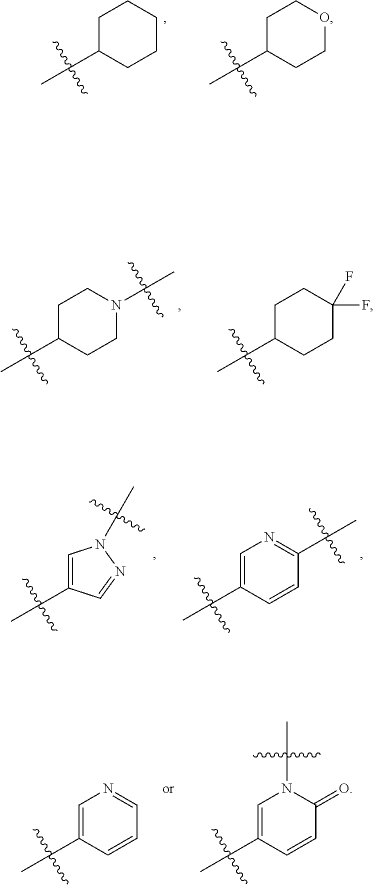

In another preferred embodiment, the compound represented by the general formula (I) is a compound represented by the general formula (Ia):

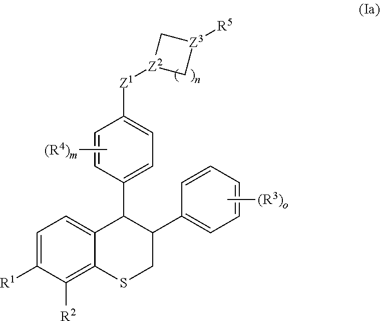

R¹, R², R³, o, R⁴, m, Z¹, Z², n, Z³, and R⁵ are defined as described above.

In another preferred embodiment, the compound represented by the general formula (I) has a compound represented by the general formula (Ib):

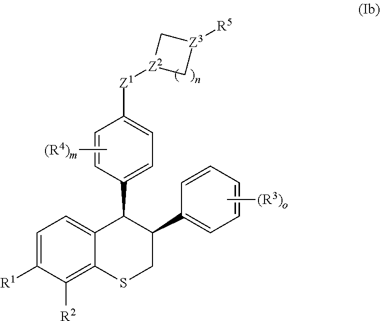

wherein, R¹, R², R³, o, R⁴, m, Z¹, Z², n, Z³, and R⁵ are defined as described above.

In another preferred embodiment, the compound is a compound prepared in Examples.

In another preferred embodiment, the pharmaceutically acceptable salt is a salt formed by a compound and an organic acid or an inorganic acid, wherein the organic acid is selected from L-tartaric acid, fumaric acid, L-malic acid, D-malic acid, citric acid, L-pyroglutamic acid, acetic acid, p-toluenesulfonate, benzenesulfonic acid, methanesulfonic acid, benzoic acid, lactic acid, mandelic acid, maleic acid, oxalic acid, and succinic acid; and the inorganic acid is selected from hydrochloric acid, phosphoric acid, sulfuric acid, and hydrobromic acid.

The second aspect of the present invention provides a method for preparing the compound represented by the general formula (I) described in the first aspect.

When R¹ is OH, R² is hydrogen, Z¹ is OCH₂CH₂, and Z² is N, the preparation method comprises the following steps:

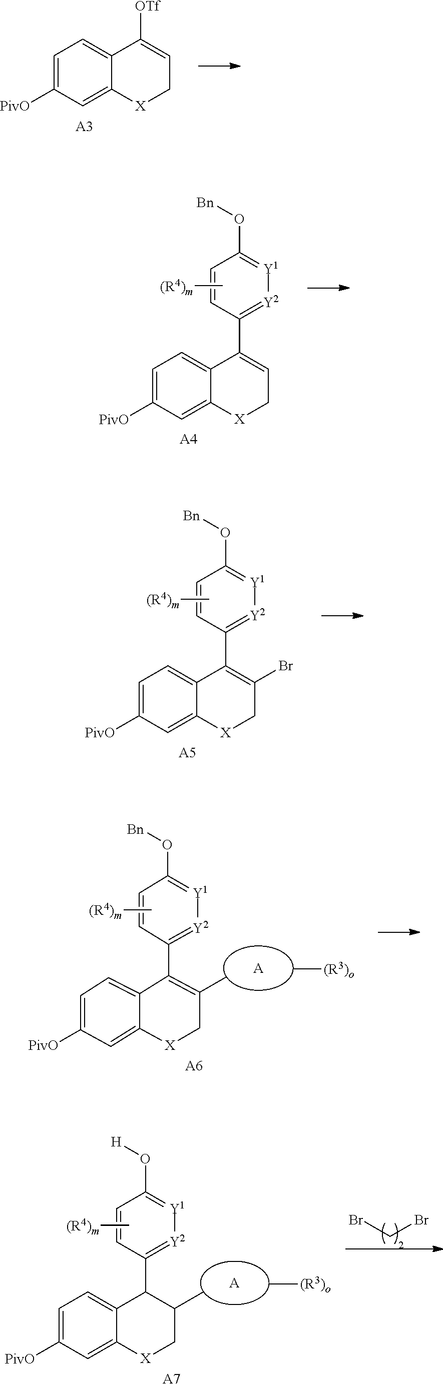

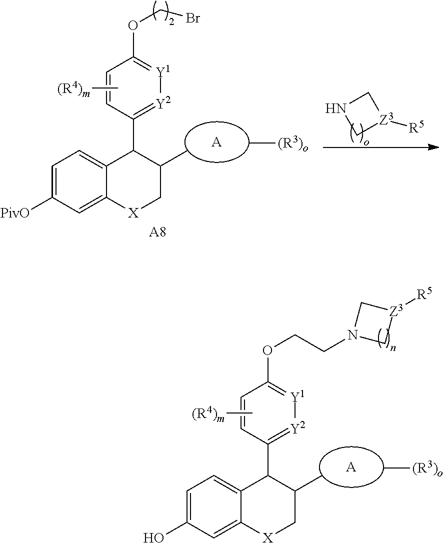

(i1) reacting A3 with an organic boron reagent through Suziki coupling reaction to obtain A4, wherein the organic boron reagent is selected from: boric acid

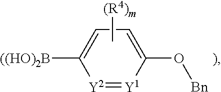

boric acid pinacol ester

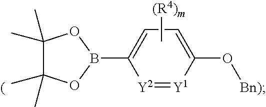

(i2) reacting A4 with a bromination reagent through bromination reaction to obtain an alkenyl bromide A5, wherein the bromination reagent is selected from: pyridinium tribromide, N-bromosuccinimide;
(i3) reacting A5 with an organic boron reagent through Suziki coupling reaction to obtain A6, wherein the organic boron reagent is selected from: boric acid

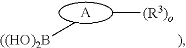

boric acid pinacol ester

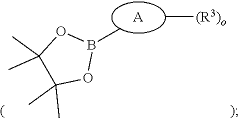

(i4) subjecting A6 to hydrogenation and hydrogenolysis reaction under the action of a palladium catalyst and hydrogen to obtain A7, wherein the palladium catalyst is selected from Pd/C and Pd(OH)₂/C;
(i5) subjecting A7 to nucleophilic substitution reaction to obtain A8;
(i6) subjecting A8 to nucleophilic substitution reaction and hydrolysis under alkaline condition to obtain a compound of formula (I), wherein the base is selected from triethylamine, N,N-diisopropylethylamine, pyridine, carbonate, NaH, sodium hydroxide, potassium hydroxide, lithium hydroxide, sodium methoxide, and sodium ethoxide;
X, R⁴, Y², Y¹, ring A, R³, o, m, Z³, and R⁵ are as defined above;
or the preparation method comprises the following steps:

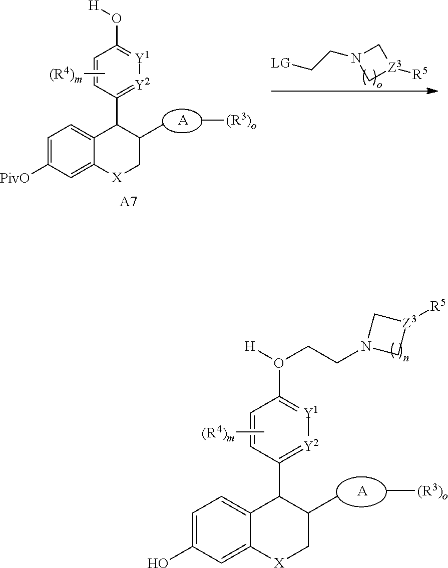

wherein, reacting A7 with

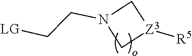

to obtain a compound of formula (I),
LG is a leaving group selected from Br, Cl, OTf, OTs or OMs;
X, R⁴, Y², Y¹, ring A, R³, o, m, Z³, and R⁵ are as defined above;
or R¹ is —COOH, R² is hydrogen, and the preparation method comprises the following steps:

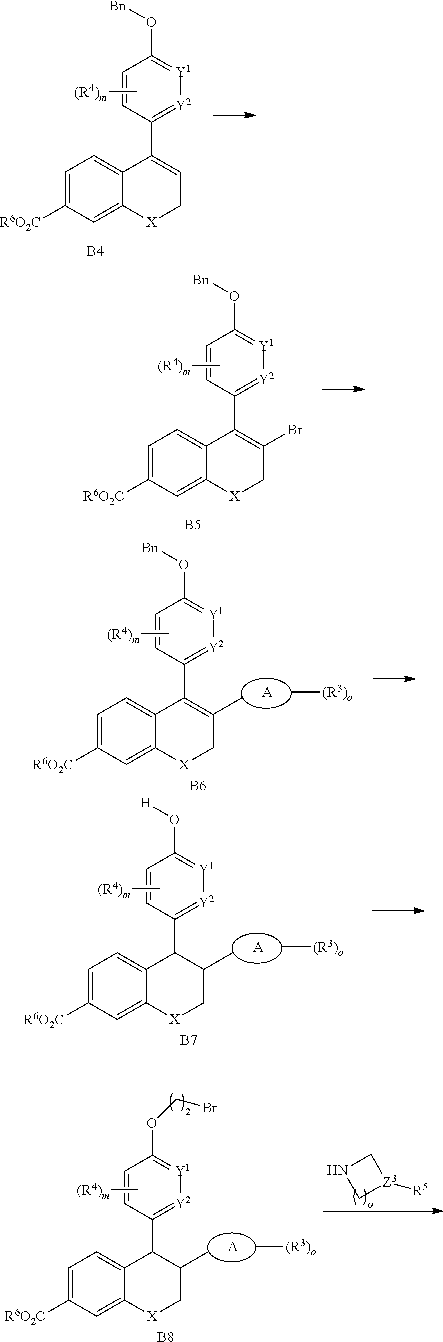

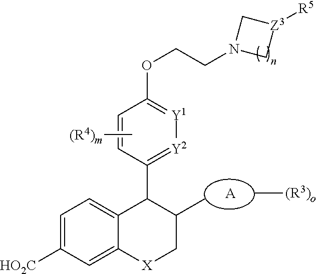

(ii1) reacting B4 with a bromination reagent through bromination reaction to obtain an alkenyl bromide B5, wherein the bromination reagent is selected from: pyridinium tribromide, N-bromosuccinimide;
(ii2) reacting B5 with an organic boron reagent through Suziki coupling reaction to obtain B6, wherein the organic boron reagent is selected from boric acid

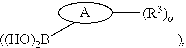

boric acid pinacol ester

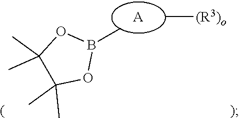

(ii3) subjecting B6 to hydrogenation and hydrogenolysis reaction under the action of a palladium catalyst and hydrogen to obtain B7, wherein the palladium catalyst is selected from Pd/C and Pd(OH)₂/C;
(ii4) subjecting B7 to nucleophilic substitution reaction to obtain B8;
(ii5) subjecting B8 to nucleophilic substitution reaction and hydrolysis under alkaline conditions to obtain a compound of formula (I), wherein the base is selected from triethylamine, N,N-diisopropylethylamine, pyridine, carbonate, NaH, sodium hydroxide, potassium hydroxide, lithium hydroxide, sodium methoxide, and sodium ethoxide;
X, R⁴, Y², Y¹, ring A, R³, o, m, Z³, R⁵ are as defined above, and R⁶ is C₁-C₆ alkyl;
or when R¹, R² and the connected benzene ring form

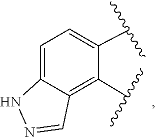 [SMILES: *c1ccc2[nH]ncc2c1*]

the preparation method comprises the following steps:

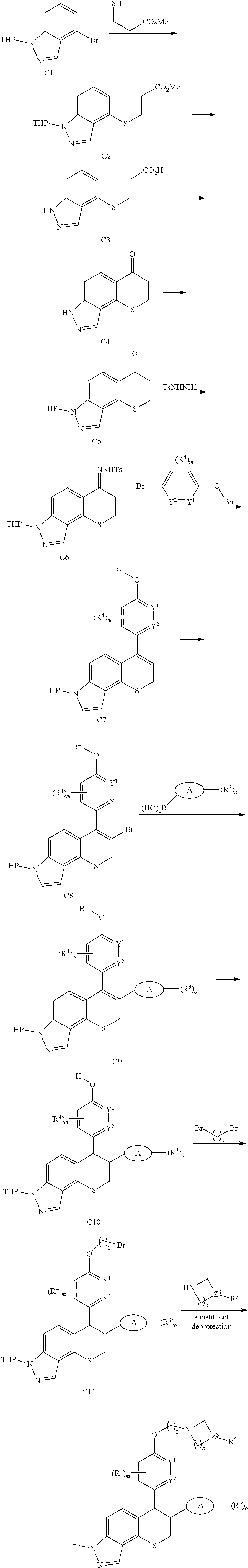

(iii1) reacting C1 and methyl 3-mercaptopropionate through C—S coupling under the action of a palladium catalyst to obtain C2, wherein the palladium catalyst is selected from [1,1′-bis(diphenylphosphino)ferrocene]palladium dichloride, tetrakis(triphenylphosphine) palladium, tris(dibenzylideneacetone)dipalladium, bis(dibenzylideneacetone)palladium, bistriphenylphosphinepalladium dichloride, di(tri-tert-butylphosphine)palladium, bis(tricyclohexylphosphine)palladium, and palladium acetate;
(iii2) hydrolyzing C2 under acidic conditions to obtain C3, wherein the acid is selected from sulfuric acid, hydrochloric acid, phosphoric acid, methanesulfonic acid, trifluoroacetic acid, acetic acid, and trifluoromethanesulfonic acid;
(iii3) subjecting C3 to Friedel-Crafts reaction under the action of an acid to obtain C4, wherein the acid is selected from the group consisting of trifluoromethanesulfonic acid, trifluoroacetic acid, Eaton's reagent, polyphosphoric acid, sulfuric acid, and hydrochloric acid;
(iii4) reacting C4 with dihydropyran in the presence of an acid to obtain C5, wherein the acid includes p-toluenesulfonic acid, methanesulfonic acid, and p-toluenesulfonic acid pyridinium salt;
(iii5) reacting C5 with p-toluenesulfonyl hydrazide to obtain hydrazone C6;
(iii6) reacting C6 with an aromatic bromide in the presence of a palladium catalyst to obtain C7, wherein the palladium catalyst is selected from [1,1′-bis(diphenylphosphino)ferrocene]palladium dichloride, tetrakis(triphenylphosphine)palladium, tris(dibenzylideneacetone)dipalladium, bis(dibenzylideneacetone)palladium, bistriphenylphosphinepalladium dichloride, di(tri-tert-butylphosphine)palladium, bis(tricyclohexylphosphine)palladium, and palladium acetate;
(iii7) reacting C7 with a bromination reagent through bromination reaction to obtain an alkenyl bromide C8, wherein the bromination reagent is selected from: pyridinium tribromide, N-bromosuccinimide;
(iii8) reacting C8 with an organic boron reagent through Suziki coupling reaction to obtain C9, wherein the organic boron reagent is selected from: boric acid

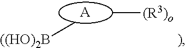

boric acid pinacol ester

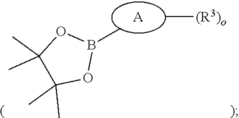

(iii9) subjecting C9 to hydrogenation and hydrogenolysis reaction under the action of a palladium catalyst and hydrogen to obtain C10, wherein the palladium catalyst is selected from Pd/C and Pd(OH)₂/C;
(iii10) subjecting C10 to nucleophilic substitution reaction to obtain C11;
(iii11) subjecting C11 to nucleophilic substitution reaction and hydrolysis under acidic conditions to obtain a compound of formula (I), wherein the acid is selected from trifluoromethanesulfonic acid, trifluoroacetic acid, sulfuric acid, hydrochloric acid, p-toluenesulfonic acid, and a solution of hydrogen chloride in an organic solvent (methanol, ethanol, isopropanol, ethyl acetate, diethyl ether or 1,4-dioxane);
X, R⁴, Y², Y¹, ring A, R³, o, m, Z³, and R⁵ are defined as above.

The third aspect of the present invention provides a pharmaceutical composition comprising:
the compound represented by the general formula (I) described in the first aspect, or the racemate, enantiomer, diastereomer, or mixture thereof, or pharmaceutically acceptable salt thereof; and a pharmaceutically acceptable carrier.

In another preferred embodiment, the pharmaceutical composition further comprises a glidant or a diluent.

The fourth aspect of the present invention provides use of the compound represented by the general formula (I) described in the first aspect, or the racemate, enantiomer, diastereoisomer, or mixture thereof, or pharmaceutically acceptable salt thereof, or the pharmaceutical composition described in the third aspect, for preparing a drug for treating, preventing or diagnosing estrogen receptor-related diseases, preferably, for preparing a drug for treating, preventing or diagnosing breast cancer, endometrial cancer, cervical cancer, skin cancer, prostate cancer, ovarian cancer, fallopian tube tumor, lung cancer, leukemia, osteoporosis, neurodegenerative disease, cardiovascular disease, lupus erythematosus, endometriosis and obesity.

In another preferred embodiment, the disease associated with estrogen receptor is an estrogen receptor-sensitive, estrogen receptor-mediated or dependent disease or condition.

In another preferred embodiment, the disease associated with estrogen receptor is selected from cancer, osteoporosis, neurodegenerative disease, cardiovascular disease, lupus erythematosus, endometriosis and obesity.

In another preferred embodiment, the disease associated with estrogen receptor is selected from breast cancer, endometrial cancer, cervical cancer, skin cancer, prostate cancer, ovarian cancer, fallopian tube tumor, lung cancer and leukemia.

In another preferred embodiment, the disease associated with estrogen receptor is selected from ER-positive breast cancer.

In another preferred embodiment, the disease associated with estrogen receptor is selected from ER-positive breast cancer with brain metastasis.

The present invention provides a SERD with novel structure, excellent ER antagonism and degradation activity, and oral efficacy, which can treat, prevent or diagnose estrogen receptor-sensitive, estrogen receptor-mediated or dependent diseases or conditions, especially estrogen receptor-positive breast cancer. In addition, the compound of the present invention has a good brain exposure and can be used for the treatment of patients with brain metastatic breast cancer, meeting the huge unmet clinical need.

It should be understood that within the scope of the present invention, the above-mentioned technical features of the present invention and the technical features specifically described below (such as examples) can be combined with each other to form a new or preferred technical solution. Each feature disclosed in the specification can be replaced by any alternative feature that provides the same, equal or similar purpose. Due to space limitations, they will not be described one by one here.

### BRIEF DESCRIPTION OF THE DRAWINGS

FIG. 1 is a graph showing the results of the uterine wet weight experiment, wherein A shows the average ratio of uterine weight to body weight in different experimental groups, *p<0.05, **p<0.01; B is a histological image of the cross-section of the uterus of each experimental group, and the height of the endometrial surface epithelium is marked with a black line.

FIG. 2 is a graph showing the results of growth inhibition experiments of compounds on subcutaneous tumor models in MCF-7 mice, **p<0.01.

### DETAILED DESCRIPTION OF THE INVENTION

The inventors of the present application have developed a thiochroman derivative through extensive and in-depth research, having a structure shown in the general formula (I), as a selective estrogen receptor degrader and antagonist, for the treatment of estrogen receptor positive diseases, particularly estrogen receptor positive breast cancer. In addition, the compound of the present invention has a good brain exposure and can be used for the treatment of patients with brain metastatic breast cancer. On this basis, the present invention was completed.

### Term

Unless stated to the contrary, the following terms used in the specification and claims have the following meanings.

In the present invention, the term “C₁-C₆” refers to having 1, 2, 3, 4, 5 or 6 carbon atoms, “C₁-C₄” refers to having 1, 2, 3 or 4 carbon atoms, and so on. “3-6 membered” refers to having 3, 4, 5 or 6 ring atoms, “5-8 membered” refers to having 5, 6, 7 or 8 ring atoms, and so on.

“Hydrogen”, “carbon” and “oxygen” in the compounds of the present invention include all isotopes thereof. Isotopes should be understood to include atoms having the same atomic number but different mass numbers. For example, hydrogen isotopes include tritium and deuterium, carbon isotopes include ¹³C and ¹⁴C, oxygen isotopes include ¹⁶O and ¹⁸O, and so on.

“Halogen” refers to fluorine, chlorine, bromine or iodine; “halo” refers to fluoro, chloro, bromo or iodo.

“Cyano” refers to —CN.

“Carboxy” refers to —C(═O)OH.

“Alkyl” means the group formed by a saturated hydrocarbon consisting of only two elements, C and H, after the loss of a hydrogen atom at any carbon atom, including straight and branched aliphatic hydrocarbons, non-limiting examples of which include methyl, ethyl, n-propyl, isopropyl, n-butyl, isobutyl, tert-butyl, sec-butyl.

“Alkylene” refers to a straight or branched saturated aliphatic group having a specified number of carbon atoms and connecting at least two other groups, i.e., a divalent hydrocarbon group. The two groups connected to the alkylene group can be connected to the same or different atoms on the alkylene group. For example, a straight chain alkylene can be a divalent group of —(CH₂)ₙ—, where n is 1, 2, 3, 4, 5 or 6. Representative alkylene includes, but is not limited to, methylene, ethylene, propylene, isopropylene, butylene, isobutylene, sec-butylene, pentylene and hexylene. The alkylene can be substituted or unsubstituted.

“Alkenyl” refers to an alkyl group as defined above consisting of at least two carbon atoms and at least one carbon-carbon double bond, for example, ethenyl, 1-propenyl, 2-propenyl, and the like.

“Cycloalkyl” refers to a saturated or partially unsaturated monocyclic or polycyclic hydrocarbyl. Polycyclic cycloalkanes include spiro, fused and bridged cycloalkyls.

“Alkoxy” refers to —O-(alkyl) and —O-(cycloalkyl), wherein alkyl and cycloalkyl are as described above.

“Alkylthio” refers to —S-(alkyl) and —S-(cycloalkyl) wherein alkyl and cycloalkyl are as previously described.

In the present invention, the term “aryl” means a hydrocarbyl comprising one or more aromatic rings. For example, the term “C6-C12 aryl” refers to an aromatic ring radical having 6 to 12 carbon atoms without heteroatoms on the ring, such as phenyl, naphthyl, etc. The term “C6-C10 aryl” has a similar meaning. Examples of aryl include, but are not limited to, phenyl (Ph), naphthyl, pyrenyl, anthracenyl, and phenanthrenyl.

In the present invention, the term “heterocyclyl” refers to a saturated or unsaturated, non-aromatic cyclic group containing at least one (such as 1, 2, 3 or 4) ring heteroatom (such as N, O or S), for example piperidinyl, tetrahydropyranyl, tetrahydropyridinyl, pyrrolinyl, dihydropyridinyl, dihydrofuranyl, dihydrothiophenyl, morpholinyl.

In the present invention, the term “heteroaryl” refers to an aromatic cyclic group containing at least one (such as 1, 2, 3 or 4) ring heteroatoms (such as N, O or S), for example furanyl, pyrrolyl, thienyl, oxazolyl, imidazolyl, thiazolyl, pyridyl, quinolyl, isoquinolyl, indolyl, pyrimidinyl, pyranyl.

“Substituted” means that one or more hydrogen atoms in a group are independently replaced by a corresponding number of substituents; the substituted is mono-substituted or poly-substituted, preferably, the poly-substituted is di-substituted, tri-substituted, tetra-substituted, or penta-substituted. The di-substituted means having two substituents, and so on.

“Independently” means that when there are more than one substituent, these substituents may be the same or different.

“Isomers” of the present invention refer to compounds having the same molecular formula but differing in nature or in the bond sequence of their atoms or in the spatial arrangement of their atoms. Stereoisomers are isomers whose atoms differ in spatial arrangement. Stereoisomers that are not mirror images of each other are diastereomers and stereoisomers that are non-superimposable mirror images of each other are enantiomers. Chiral compounds can exist as a single enantiomer or as a mixture thereof. A mixture containing equal proportions of enantiomers is called a “racemic mixture”.

The “medicinal salt” and “pharmaceutically acceptable salt” of the present invention refer to the salts of the compounds of the present invention, which are safe and effective when used in mammals and have the desired biological activity. In another preferred embodiment, “medicinal salt” and “pharmaceutically acceptable salt” refer to salts formed by the compound of formula I with an acid selected from the group consisting of hydrofluoric acid, hydrochloric acid, hydrobromic acid, phosphoric acid, acetic acid, oxalic acid, sulfuric acid, nitric acid, methanesulfonic acid, aminosulfonic acid, salicylic acid, trifluoromethanesulfonic acid, naphthalenesulfonic acid, maleic acid, citric acid, acetic acid, lactic acid, tartaric acid, succinic acid, oxalic acid, pyruvic acid, malic acid, glutamic acid, p-toluenesulfonic acid, naphthalenesulfonic acid, ethanesulfonic acid, naphthalenedisulfonic acid, malonic acid, fumaric acid, propionic acid, oxalic acid, trifluoroacetic acid, stearic acid, pamoic acid, hydroxymaleic acid, phenylacetic acid, benzoic acid, glutamic acid, ascorbic acid, p-aminobenzenesulfonic acid, 2-acetoxybenzoic acid and isethionic acid; or sodium salts, potassium salts, calcium salts, aluminum salts or ammonium salts formed by the compound of formula I with an inorganic base; or methylamine salts, ethylamine salts or ethanolamine salts formed by the compound of general formula I with an organic base.

### Detailed Embodiment

The present invention is completed by conducting extensive compound design, synthesis, in vitro and in vivo biological activity testing, metabolism and other druggability studies. The following examples are only specific descriptions of the present invention and do not constitute any limitation to the present invention. Those skilled in the art use the techniques, methods, or permutations and combinations of related methods known in the art, and minor changes made to the present invention are all within the scope of protection of the present invention.

### Synthesis Method of the Compound of the Present Invention

The structure of the compound of the present invention is determined by nuclear magnetic resonance (NMR) or mass spectrometry (MS). NMR data are collected on a BRUKER AVANCE III 400 or BRUKER AVANCE III 500 or BRUKER AVANCE III 600 nuclear magnetic resonance spectrometer, and the measuring solvent is deuterated dimethyl sulfoxide (DMSO-d₆), deuterated chloroform (CDCl₃) or deuterated methanol (CD₃OD). The chemical shift is expressed in δ (ppm), and the abbreviations used to describe the peak signal are as follows: br=broad signal, s=single peak, d=double peak, dd=double double peak peak, t=triplet, q=quartet, m=multiple peak. The mass spectrum is measured using a Finnigan LTQ linear ion trap mass spectrometer. The silica gel used for column chromatography separation is 200-300 mesh, and the ratio of the eluent is all volume ratio. The commercial raw materials, reagents (such as acids, bases, metal catalysts, bromination reagents, acylating reagents, arylboronic acids, etc.), solvents (petroleum ether, ethyl acetate, methanol, dichloromethane, 1,4-dioxane, acetonitrile, etc.) used in the synthesis were purchased from reagent companies and used directly in the reaction without additional purification.

In a preferred example of the compound of the general formula (I) of the present invention, X is S, R¹ is OH, R² is hydrogen, Z¹ is —OCH₂CH₂—, and Z² is N; the compound represented by the general formula (I), the racemate, enantiomer, diastereomer and mixture thereof, and the pharmaceutically acceptable salt thereof can be prepared by the following route 1:

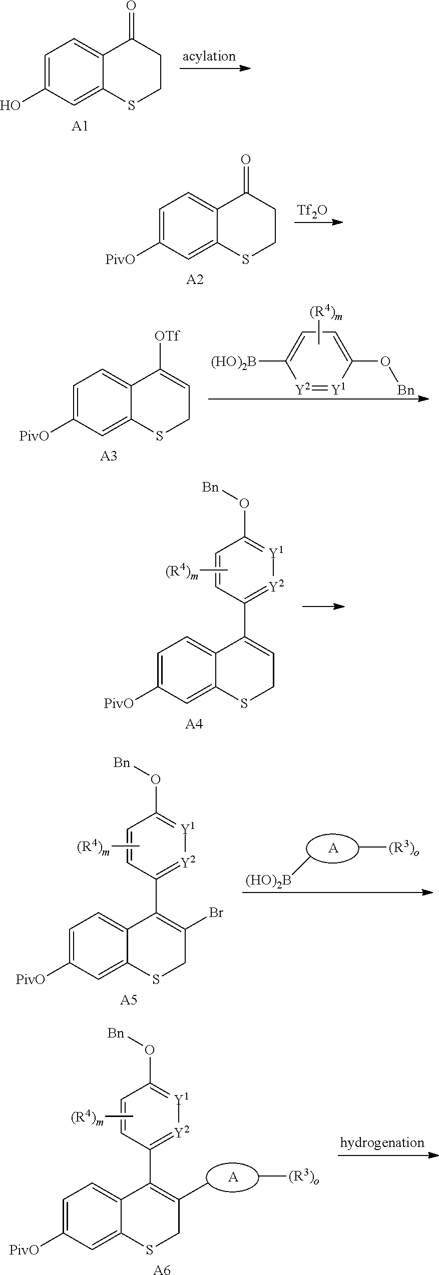

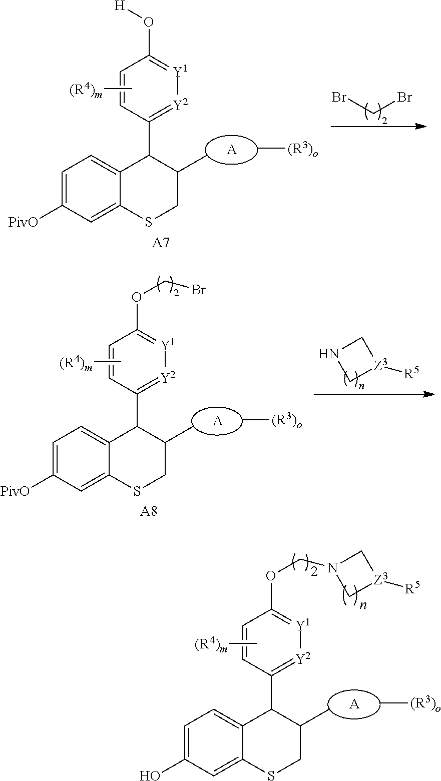

wherein R³, R⁴, R⁵, Z³, m, n, o are as described above;
step 1: A1 is reacted with PivCl in the presence of a base to obtain A2, wherein the base includes triethylamine, N,N-diisopropylethylamine, pyridine, or carbonate, and the solvent includes chlorinated hydrocarbons, ethers, esters, or acetonitrile, and the temperature is 0-50° C.;
step 2: A2 is reacted with trifluoromethanesulfonic anhydride under the action of pyridine to obtain trifluoromethanesulfonate A3, the solvent includes chlorinated hydrocarbons, ethers, and the temperature is 0° C. to room temperature;
step 3: A3 is reacted with an aryl boron reagent through Suzuki coupling in the presence of a palladium catalyst and a base to obtain A4, wherein the palladium catalyst includes but is not limited to [1,1′-bis(diphenylphosphino)ferrocene]palladium dichloride, tetrakis(triphenylphosphine)palladium, tri(dibenzylideneacetone)dipalladium, bis(dibenzylideneacetone)palladium, bistriphenylphosphinepalladium dichloride, di(tri-tert-butylphosphine)palladium, bis(tricyclohexylphosphine)palladium, palladium acetate, the aryl boron reagent includes but is not limited to boric acid, boric acid pinacol ester, boric acid neopentyl glycol ester, the base includes but is not limited to potassium acetate, sodium carbonate, potassium carbonate, cesium carbonate, 1,8-diazabicyclo[5.4.0]undec-7-ene, triethylamine, N,N-diisopropylethylamine, and the solvent includes water, 1,4-dioxane, toluene, N,N-dimethylformamide, acetonitrile, ethanol, and methanol;
step 4: A4 is reacted with a brominating agent to obtain an alkenyl bromide A5, wherein the brominating agent includes pyridinium tribromide, N-bromosuccinimide, and bromine, and the solvent includes a halogenated hydrocarbon, acetonitrile, acetic acid, and methanol;
step 5: A5 is reacted with an organic boron reagent through Suzuki coupling in the presence of a palladium catalyst and a base to obtain A6, wherein the palladium catalyst includes but is not limited to [1,1′-bis(diphenylphosphino)ferrocene]palladium dichloride, tetrakis(triphenylphosphine)palladium, tri(dibenzylideneacetone)dipalladium, bis(dibenzylideneacetone)palladium, bistriphenylphosphinepalladium dichloride, di(tri-tert-butylphosphine)palladium, bis(tricyclohexylphosphine)palladium, palladium acetate, the aryl boron reagent includes but is not limited to boric acid, boric acid pinacol ester, boric acid neopentyl glycol ester, the base includes but is not limited to potassium acetate, sodium carbonate, potassium carbonate, cesium carbonate, 1,8-diazabicyclo[5.4.0]undec-7-ene, triethylamine, N,N-diisopropylethylamine, and the solvent includes water, 1,4-dioxane, toluene, N,N-dimethylformamide, acetonitrile, ethanol, and methanol;
step 6: A6 is hydrogenated and hydrogenolyzed under the action of Pd/C or Pd(OH)₂/C and hydrogen to obtain A7, wherein the solvent includes dioxane, tetrahydrofuran, toluene, N,N-dimethylformamide, dimethyl sulfoxide, dichloromethane, dichloroethane, ethanol, methanol, 1-50 atmospheres, and the temperature is 0-50° C.;
step 7: A7 is reacted with dibromoethane in the presence of a base to obtain A8, wherein the base includes triethylamine, N,N-diisopropylethylamine, pyridine, carbonate, NaH, sodium hydroxide, potassium hydroxide, lithium hydroxide, and the solvent includes dioxane, tetrahydrofuran, toluene, acetonitrile, acetone, dichloromethane, dichloroethane, ethanol, methanol, and the temperature is 20-80° C.;
step 8: A8 is reacted with a corresponding amine through an amination reaction in the presence of a base and the protecting group is removed to obtain a compound of formula (I), wherein the base includes triethylamine, N,N-diisopropylethylamine, pyridine, carbonate, NaH, sodium hydroxide, potassium hydroxide, lithium hydroxide, and the solvent includes 1,4-dioxane, tetrahydrofuran, toluene, acetonitrile, acetone, dichloromethane, dichloroethane, ethanol, methanol, and the temperature is 20-80° C.

In a preferred example of the compound of the general formula (I) of the present invention, X is S, R¹ is COOH, R² is hydrogen, Z¹ is —OCH₂CH₂—, and Z² is N; the compound represented by the general formula (I), the racemate, enantiomer, diastereomer and mixture thereof, and the pharmaceutically acceptable salt thereof can be prepared by the following route 2:

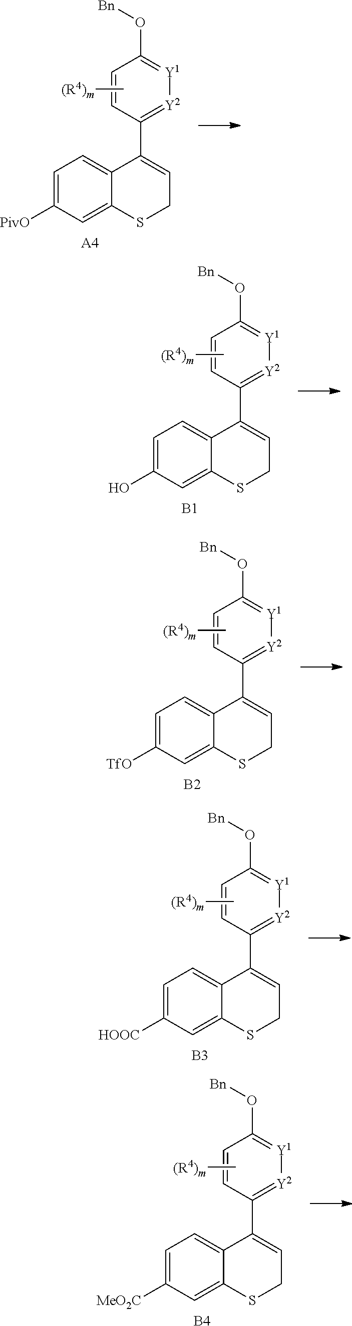

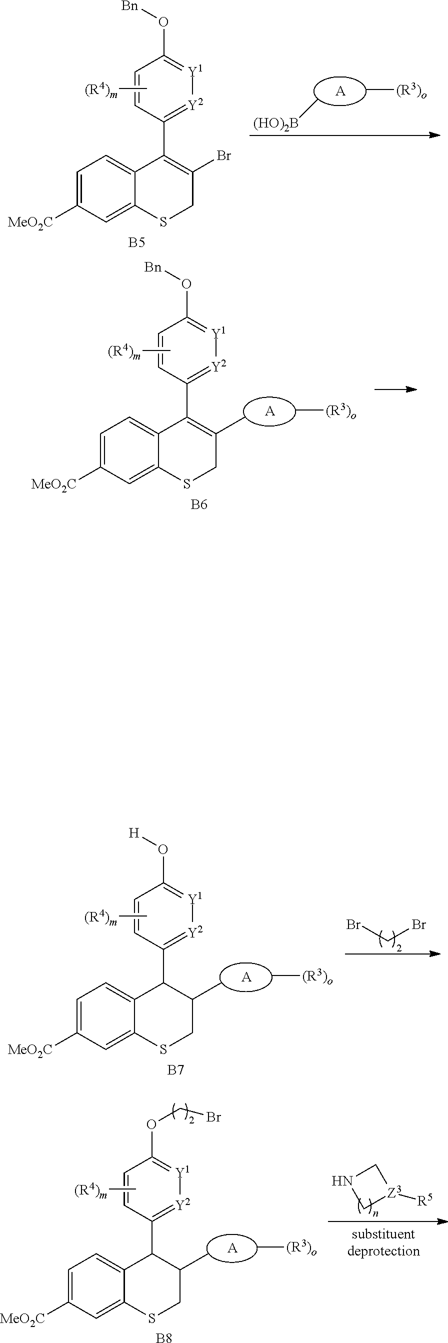

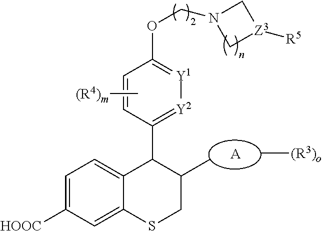

wherein R³, R⁴, R⁵, Z³, m, n, and o are as described above;
step 1: A1 is deprotected under the action of a base to obtain B2, wherein the base includes sodium hydroxide, potassium hydroxide, lithium hydroxide, sodium methoxide, sodium ethoxide, and the solvent includes water, 1,4-dioxane, tetrahydrofuran, ethanol, methanol, and the temperature is 20-80° C.;
step 2: B1 is reacted with trifluoromethanesulfonic anhydride under the action of a base to obtain trifluoromethanesulfonate B2, wherein the base includes triethylamine, N,N-diisopropylethylamine, pyridine, carbonate, NaH, and the solvent includes 1,4-dioxane, tetrahydrofuran, dichloromethane, dichloroethane, ethanol, methanol, and the temperature is 0-50° C.;
step 3: B2 is reacted with a carboxylating agent through carboxylation reaction under the action of a palladium catalyst to obtain B3, wherein the palladium catalyst includes but is not limited to [1,1′-bis(diphenylphosphino)ferrocene]palladium dichloride, tetrakis(triphenylphosphine)palladium, tris(dibenzylideneacetone)dipalladium, bis(dibenzylideneacetone)palladium, bistriphenylphosphinepalladium dichloride, di(tri-tert-butylphosphine)palladium, bis(tricyclohexylphosphine)palladium, palladium acetate, the carboxylating agent includes formic acid, sodium formate, potassium formate, lithium formate; the solvent includes 1,4-dioxane, tetrahydrofuran, toluene, N,N-dimethylformamide, dimethyl sulfoxide, acetonitrile, and the temperature is 50-100° C.;
step 4: B3 is subjected to esterification reaction to obtain B4, wherein the conditions include methanol/concentrated sulfuric acid, iodomethane/base, TMSCHN₂/methanol, wherein the base includes NaH, carbonate, sodium hydroxide, potassium hydroxide;
step 5-8: the compound of formula (I) is prepared from B4 by referring to the method for preparing the compound of formula (I) from A4 in route 1.

In another preferred embodiment of the compound of the general formula (I) of the present invention, R¹ and R² in the compound of the general formula (I) form pyrazole

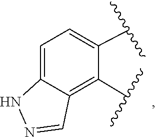

the compound of the general formula (I) and the racemate, enantiomer, diastereomer and mixture thereof, and pharmaceutically acceptable salt thereof can be prepared by route 3:

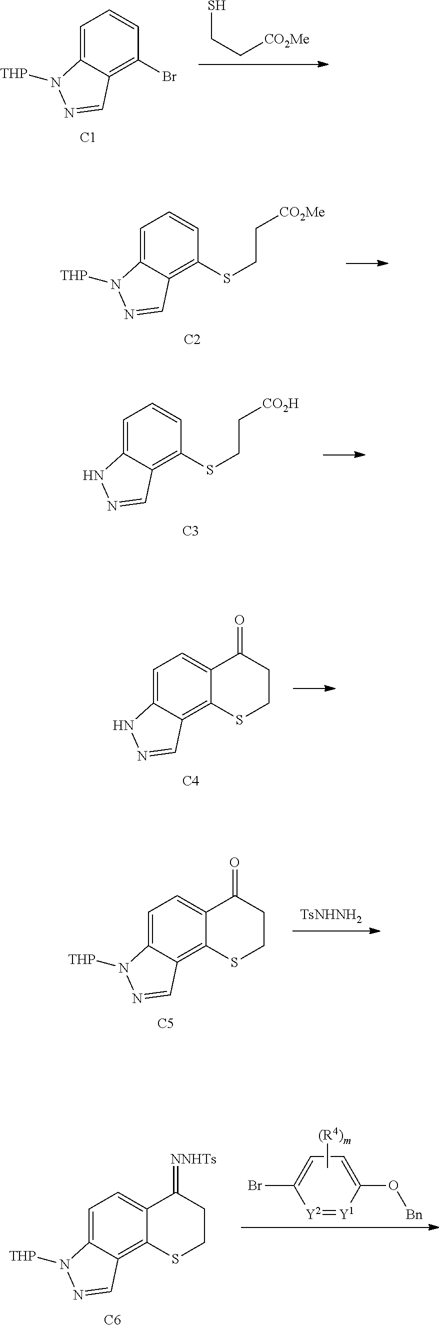

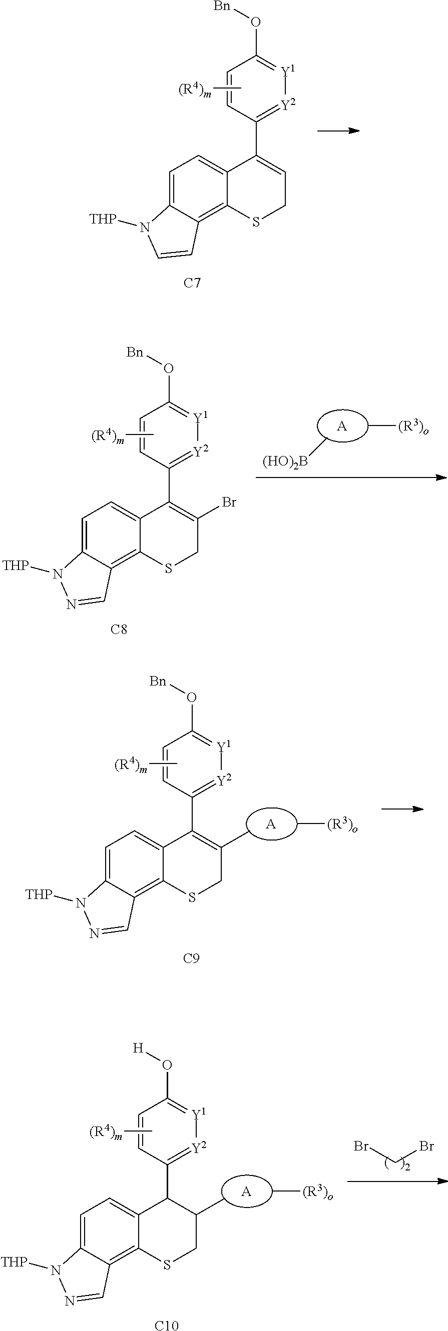

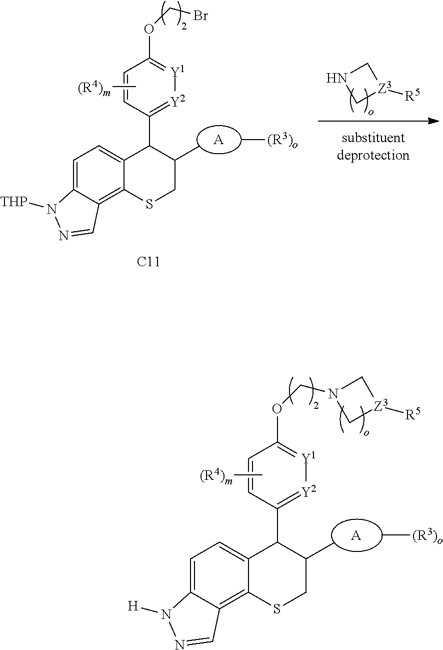

wherein R³, R⁴, R⁵, Z³, m, n, and o are as described above;
step 1: C1 is reacted with methyl 3-mercaptopropionate under the action of a palladium catalyst and a base to obtain C2, wherein the palladium catalyst includes but is not limited to [1,1′-bis(diphenylphosphino)ferrocene]dichloropalladium, tetrakis(triphenylphosphine)palladium, tri(dibenzylideneacetone)dipalladium, bis(dibenzylideneacetone)palladium, bistriphenylphosphinepalladium dichloropalladium, di(tri-tert-butylphosphine)palladium, bis(tricyclohexylphosphine)palladium, palladium acetate; the aryl boron reagent includes but is not limited to boric acid, boric acid pinacol ester, boric acid neopentyl glycol ester, and the base includes but is not limited to potassium acetate, sodium carbonate, potassium carbonate, cesium carbonate, 1,8-diazabicyclo[5.4.0]undec-7-ene, triethylamine, N,N-diisopropylethylamine;
step 2: C2 is hydrolyzed under an acidic condition to obtain C3, wherein the acid includes sulfuric acid, hydrochloric acid, phosphoric acid, methanesulfonic acid, trifluoroacetic acid, acetic acid, trifluoromethanesulfonic acid, and the solvent includes water, 1,4-dioxane, tetrahydrofuran, toluene, acetonitrile, acetone, and the temperature is room temperature −100° C.;
step 3: C3 undergoes Friedel-Crafts reaction under the action of a protonic acid to obtain C4, wherein the protonic acid includes but is not limited to polyphosphoric acid, Eaton's reagent, and trifluoromethanesulfonic acid;
step 4: C4 is reacted with 2,3-dihydropyran in the presence of p-toluenesulfonic acid or p-toluenesulfonic acid-pyridinium salt to obtain C5, wherein the solvent comprises halogenated hydrocarbon, toluene or acetonitrile;
step 5: C5 is reacted with p-toluenesulfonyl hydrazide to obtain hydrazone C6, wherein the solvent includes methanol, ethanol, toluene, and acetonitrile;
step 6: C6 is reacted with an aromatic bromide through a carbene migration insertion reaction under the action of a palladium catalyst to obtain C7, wherein the palladium catalyst includes but is not limited to [1,1′-bis(diphenylphosphino)ferrocene]palladium dichloride, tetrakis(triphenylphosphine)palladium, tris(dibenzylideneacetone)dipalladium, bis(dibenzylideneacetone)palladium, bistriphenylphosphinepalladium dichloride, di(tri-tert-butylphosphine)palladium, bis(tricyclohexylphosphine)palladium, palladium acetate, the base includes but is not limited to potassium acetate, sodium carbonate, potassium carbonate, cesium carbonate, potassium tert-butoxide, sodium tert-butoxide, lithium tert-butoxide, NaH, and the solvent includes 1,4-dioxane, toluene, N,N-dimethylformamide, acetonitrile;
steps 7-11: The subsequent route is similar to route 1.

### Preparation of Pharmaceutically Acceptable Salts of the Compounds of the Present Invention

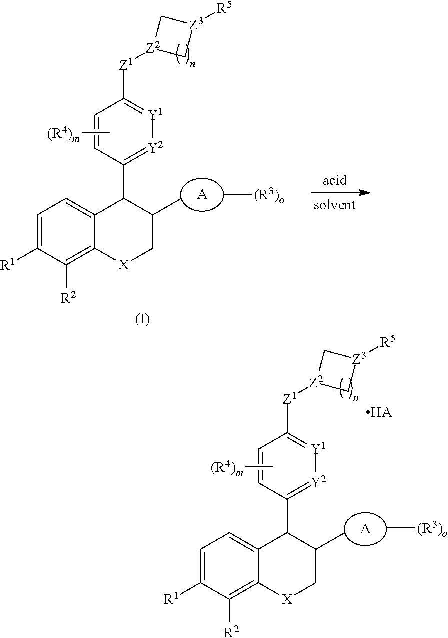

The compound of general formula (I) is dissolved in an appropriate solvent, and 1 equivalent of acid is added, or 1 equivalent of acid is dissolved in the same solvent and then the acid solution is added dropwise to the solution of the compound of general formula (I), and stirred for 1 hour. The powder is obtained after concentration (or freeze-dried), and the obtained powder is suspended in methyl tert-butyl ether, slurried for 30 minutes, filtered, and dried to obtain the corresponding salt.

The acid used is an organic acid or an inorganic acid, wherein the inorganic acid includes but is not limited to hydrochloric acid, phosphoric acid, sulfuric acid, hydrobromic acid, and nitric acid; the organic acid includes but is not limited to L-tartaric acid, fumaric acid, L-malic acid, D-malic acid, citric acid, L-pyroglutamic acid, acetic acid, p-toluenesulfonic acid, benzenesulfonic acid, methanesulfonic acid, benzoic acid, lactic acid, mandelic acid, maleic acid, oxalic acid, and succinic acid.

The solvent includes, but is not limited to, acetonitrile, acetone, ethyl acetate, dichloromethane, tetrahydrofuran, methanol, ethanol, isopropanol, 1,4-dioxane, chloroform, and ether.

The present invention is further illustrated below by means of specific preparation examples and test examples.

### Example 1

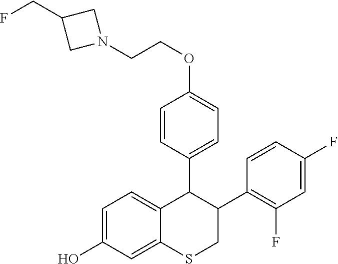 [SMILES: Oc1ccc2c(c1)SCC(c1ccc(F)cc1F)C2c1ccc(OCCN2CC(CF)C2)cc1]

### Cis-3-(2,4-difluorophenyl)-4-(4-(2-(3-(fluoromethyl)azetidin-1-yl)ethoxy)phenyl)-7-hydroxythiochroman

### Synthetic Route:

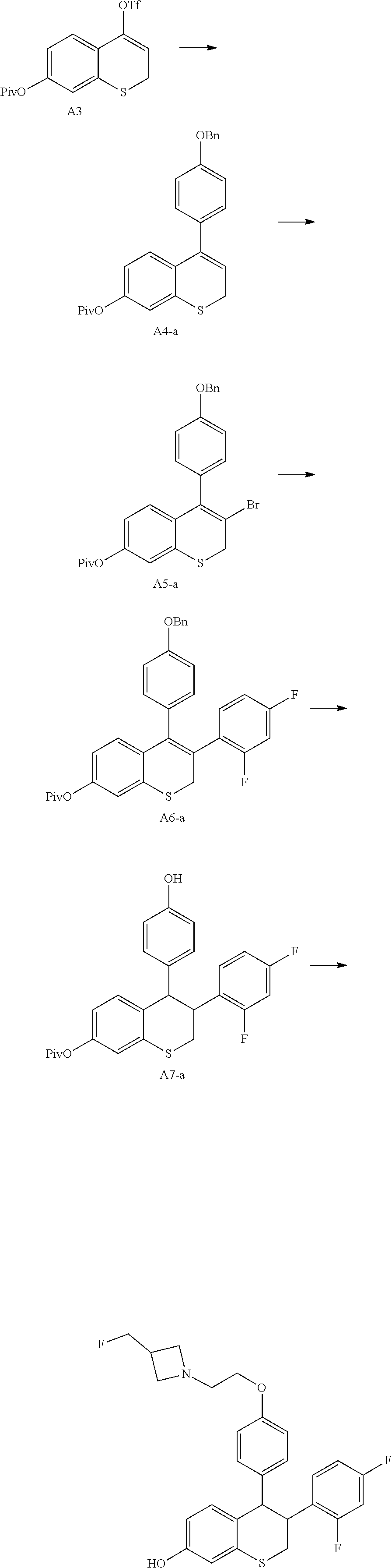

### Step 1

A3 (6.2 g, 15.7 mmol) (reference patent: WO2018091153), 4-benzyloxyphenylboronic acid (4.3 g, 18.8 mmol), Pd(dppf)Cl₂ (0.65 g, 0.8 mmol) and cesium carbonate (10.1 g, 31.4 mmol) were weighed into a reaction flask, 1,4-dioxane-water mixture (4:1 v/v, 100 mL) was added, and the mixture was reacted in a 50° C. oil bath under nitrogen protection for 1 hour. The mixture was diluted with water (50 mL) and extract with ethyl acetate (50 mL×3). The combined organic phases was washed with saturated sodium chloride solution (100 mL), dried over anhydrous sodium sulfate, filtered, concentrated under reduced pressure to remove the solvent, and separated by column chromatography (petroleum ether/ethyl acetate/dichloromethane 10:1:1) to obtain a white solid A4-a (5.9 g, 88%). ¹H NMR (400 MHz, CDCl₃) δ 7.44 (ddd, J=24.9, 17.9, 7.0 Hz, 5H), 7.24 (d, J=8.7 Hz, 2H), 7.15 (d, J=2.5 Hz, 1H), 7.09 (d, J=8.5 Hz, 1H), 7.01 (d, J=8.4 Hz, 2H), 6.84-6.74 (m, 1H), 6.02 (t, J=5.7 Hz, 1H), 5.13 (s, 2H), 3.46 (d, J=5.7 Hz, 2H), 1.40 (s, 9H).

### Step 4

A4-a (5.9 g, 13.7 mmol) was dissolved in dichloromethane (200 mL), cooled in an ice-water bath, and tribromopyridinium salt (5.4 g, 13.7 mmol, 85% content) was added. After stirring in an ice-water bath for 1 hour, the mixture was quenched with a saturated sodium bicarbonate solution (120 mL) and then separated. The aqueous phase was extracted with dichloromethane (100 mL), the organic phases were combined, washed with a saturated sodium chloride solution (100 mL), dried over anhydrous sodium sulfate, filtered, and concentrated under reduced pressure to remove the solvent to obtain a crude product, which was separated by column chromatography (petroleum ether/ethyl acetate/dichloromethane 10:1:1) to obtain a light yellow solid A5-a (5.4 g, 77%). ¹H NMR (400 MHz, CDCl₃) δ 7.53-7.47 (m, 2H), 7.47-7.41 (m, 2H), 7.41-7.35 (m, 1H), 7.19-7.13 (m, 2H), 7.06 (dt, J=6.7, 2.2 Hz, 3H), 6.74 (d, J=8.6 Hz, 1H), 6.69 (dd, J=8.6, 2.3 Hz, 1H), 5.13 (s, 2H), 3.97 (s, 2H), 1.36 (s, 9H).

### Step 5

A5-a (0.8 g, 1.57 mmol), 2,4-difluorophenylboronic acid (0.3 g, 1.88 mmol), Pd(dppf)Cl₂ (128 mg, 0.16 mmol) and cesium carbonate (1.0 g, 3.14 mmol) were added to a reaction flask, and a mixture of 1,4-dioxane-water (4:1 v/v, 20 mL) was added, and the mixture was reacted in an oil bath at 80° C. for 4 hours under nitrogen protection. After the reaction solution was cooled, it was diluted with water (50 mL), extracted with ethyl acetate (50 mL×2), the organic phases were combined, washed with saturated sodium chloride solution (40 mL), dried over anhydrous sodium sulfate, filtered, concentrated under reduced pressure to remove the solvent, and separated by column chromatography (petroleum ether/ethyl acetate/dichloromethane 15:1) to obtain a white solid A6-a (0.75 g, 88%). ¹H NMR (400 MHz, CDCl₃) δ 7.44-7.30 (m, 5H), 7.12 (d, J=2.5 Hz, 1H), 6.95-6.82 (m, 4H), 6.82-6.77 (m, 2H), 6.77-6.69 (m, 2H), 6.57 (td, J=8.5, 2.5 Hz, 1H), 5.01 (s, 2H), 3.65 (s, 2H), 1.35 (s, 9H).

### Step 6

A6-a (0.65 g) was dissolved in ethanol (15 mL), and 20% Pd(OH)₂/C (0.5 g) was added, and the mixture was reacted under a hydrogen atmosphere (1 atm) at 60° C. for 8 h. The crude product was filtered through diatomaceous earth and concentrated under reduced pressure to remove the solvent, and then separated by column chromatography (petroleum ether/ethyl acetate 3:1) to obtain a white solid A7-a (0.44 g, 81%). ¹H NMR (400 MHz, CDCl₃) δ 6.99-6.92 (m, 2H), 6.89-6.80 (m, 1H), 6.70-6.60 (m, 2H), 6.55 (d, J=8.5 Hz, 2H), 6.45 (d, J=8.5 Hz, 2H), 6.31-6.23 (m, 1H), 5.01 (s, 1H), 4.23 (d, J=3.4 Hz, 1H), 3.88 (ddd, J=12.8, 12.8, 2.8 Hz, 1H), 3.41 (dd, J=12.4, 12.4 Hz, 1H), 2.73 (d, J=11.6 Hz, 1H), 1.35 (s, 9H).

### Step 7

A7-a (0.25 g, 0.55 mmol) was dissolved in acetonitrile (15 mL), 1,2-dibromoethane (2 mL) and potassium carbonate (380 mg, 2.75 mmol) were added, and the mixture was refluxed in an oil bath for 12 h. Insoluble matter was removed by filtration, and the solvent was removed by concentration under reduced pressure. The crude product was used directly in the next Step without purification.

The crude product obtained in the previous step was dissolved in acetonitrile (15 mL), 3-fluoromethyl-azetidine hydrochloride (103 mg, 0.83 mmol) and potassium carbonate (380 mg, 2.75 mmol) were added, and after refluxing in an oil bath for 4 h, the insoluble matter was filtered off, and the solvent was removed by concentration under reduced pressure. The crude product was dissolved in methanol (5 mL), sodium methoxide (0.5 mL) was added, and the mixture was stirred at room temperature for 10 min, and then concentrated under reduced pressure to remove the solvent. The mixture was neutralized with 1N HCl to pH 7-8, and extracted with dichloromethane (15 mL×3). The organic phases were combined, and then dried over anhydrous sodium sulfate, filtered, and concentrated under reduced pressure to remove the solvent to obtain a crude product. The crude product was separated by column chromatography (dichloromethane/methanol 30:1-10:1) to obtain Example 1 (0.11 g, total yield of three steps was 42%). ¹H NMR (500 MHz, CDCl₃) δ 6.89-6.82 (m, 1H), 6.76 (d, J=8.4 Hz, 1H), 6.69 (d, J=2.1 Hz, 1H), 6.65 (t, J=7.5 Hz, 1H), 6.54-6.40 (m, 5H), 6.30-6.24 (m, 1H), 4.52 (dd, J=47.4, 5.1 Hz, 2H), 4.19 (d, J=3.0 Hz, 1H), 3.97-3.84 (m, 3H), 3.62 (t, J=7.7 Hz, 2H), 3.39 (dd, J=12.4, 12.4 Hz, 1H), 3.28 (q, J=7.2 Hz, 2H), 3.03-2.87 (m, 3H), 2.67 (d, J=11.8 Hz, 1H).

### Example 2

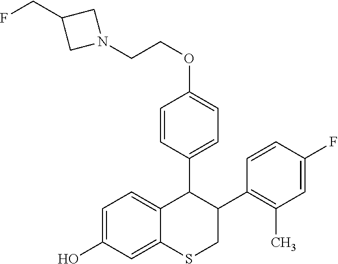 [SMILES: Cc1cc(F)ccc1C1CSc2cc(O)ccc2C1c1ccc(OCCN2CC(CF)C2)cc1]

### Cis-3-(4-fluoro-2-methylphenyl)-4-(4-(2-(3-(fluoromethyl)azetidin-1-yl)ethoxy)phenyl)-7-hydroxythiochroman

### Synthetic Route:

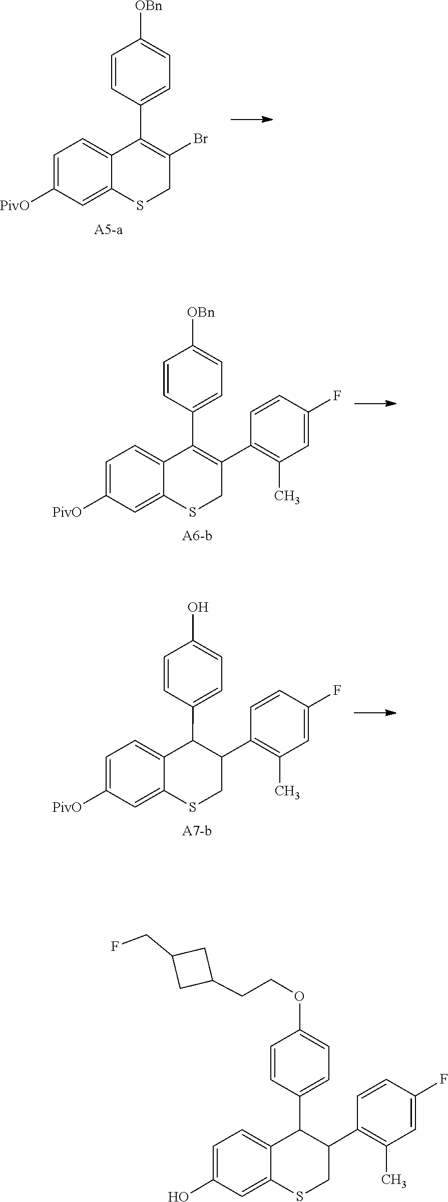

### Step 1

A5-a (0.9 g, 1.77 mmol), 4-fluoro-2-methylphenylboronic acid (0.33 g, 2.12 mmol), Pd(PPh₃)₄ (204 mg, 0.17 mmol) and cesium carbonate (1.2 g, 3.54 mmol) were added to a reaction flask, and a mixture of 1,4-dioxane-water (4:1 v/v, 20 mL) was added, and the mixture was reacted in an oil bath at 80° C. for 4 hours under nitrogen protection. After the reaction solution was cooled, it was diluted with water (50 mL), extracted with ethyl acetate (50 mL×2), the organic phases were combined, washed with saturated sodium chloride solution (40 mL), dried over anhydrous sodium sulfate, filtered, concentrated under reduced pressure to remove the solvent, and separated by column chromatography (petroleum ether/ethyl acetate/dichloromethane 15:1) to obtain a white solid A6-b (0.70 g, 74%). ¹H NMR (400 MHz, CDCl₃) δ 7.46-7.34 (m, 5H), 7.18 (d, J=2.4 Hz, 1H), 7.01-6.88 (m, 4H), 6.84-6.70 (m, 5H), 5.01 (s, 2H), 3.63 (d, J=14.3 Hz, 1H), 3.57 (d, J=14.3 Hz, 1H), 2.26 (s, 3H), 1.41 (s, 9H).

### Step 2

A6-b (0.65 g) was dissolved in ethanol (15 mL), and 20% Pd(OH)₂/C (0.5 g) was added, and the mixture was reacted under a hydrogen atmosphere (1 atm) at 60° C. for 48 h, then filtered through diatomaceous earth and concentrated under reduced pressure to remove the solvent to obtain a crude product, which was then separated by column chromatography (petroleum ether/ethyl acetate 3:1) to obtain a white solid A7-b (0.23 g, 42%). ¹H NMR (400 MHz, CDCl₃) δ 7.28 (s, 1H), 7.04-6.91 (m, 3H), 6.70 (dd, J=8.4, 2.4 Hz, 1H), 6.66-6.51 (m, 3H), 6.41 (d, J=8.3 Hz, 2H), 6.09 (dd, J=8.6, 5.9 Hz, 1H), 4.75 (s, 1H), 4.14 (d, J=2.8 Hz, 2H), 3.78-3.69 (m, 1H), 3.45 (dd, J=12.3, 12.3 Hz, 1H), 2.73 (d, J=11.7 Hz, 1H), 2.44 (s, 3H), 1.37 (s, 9H).

### Step 3

A7-b (0.22 g, 0.49 mmol) was dissolved in acetonitrile (15 mL), and 1,2-dibromoethane (2 mL) and potassium carbonate (340 mg, 2.4 mmol) were added. After the reactants were refluxed in an oil bath for 12 h, the insoluble matter was removed by filtration, and the solvent was removed by concentration under reduced pressure. The crude product was used directly in the next step without purification.

The crude product obtained in the previous step was dissolved in acetonitrile (15 mL), 3-fluoromethyl-azetidine hydrochloride (100 mg, 0.78 mmol) and potassium carbonate (340 mg, 2.4 mmol) were added, and after refluxing in an oil bath for 4 h, the insoluble matter was filtered off, and the solvent was removed by concentration under reduced pressure. The crude product was dissolved in methanol (5 mL), sodium methoxide (0.5 mL) was added, and the mixture was stirred at room temperature for 10 min, and then concentrated under reduced pressure to remove the solvent. The mixture was neutralized with 1N HCl to pH 7-8, and extracted with dichloromethane (15 mL×3). The organic phases were combined, and then dried over anhydrous sodium sulfate, filtered, and concentrated under reduced pressure to remove the solvent to obtain a crude product. The crude product was separated by column chromatography (dichloromethane/methanol 30:1-10:1) to obtain Example 2 (137 mg, total yield of three steps was 59%). ¹H NMR (500 MHz, CDCl₃) δ 6.94 (dd, J=9.7, 2.4 Hz, 1H), 6.77 (d, J=8.4 Hz, 1H), 6.70 (d, J=2.3 Hz, 1H), 6.62 (td, J=8.4, 2.4 Hz, 1H), 6.51 (d, J=8.6 Hz, 2H), 6.45 (dd, J=8.3, 2.4 Hz, 1H), 6.40 (d, J=8.4 Hz, 2H), 6.08 (dd, J=8.4, 6.0 Hz, 1H), 4.52 (dd, J=47.3, 5.3 Hz, 2H), 4.08 (d, J=3.6 Hz, 1H), 3.96-3.85 (m, 2H), 3.77-3.69 (m, 1H), 3.66-3.57 (m, 2H), 3.42 (dd, J=12.3, 12.3 Hz, 1H), 3.32-3.25 (m, 2H), 3.02-2.86 (m, 3H), 2.65 (d, J=11.7 Hz, 1H), 2.44 (s, 3H).

### Example 3

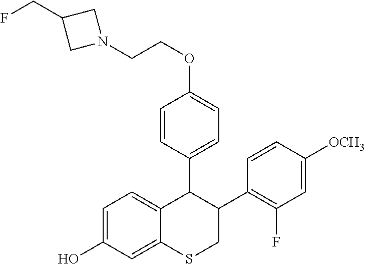 [SMILES: COc1ccc(C2CSc3cc(O)ccc3C2c2ccc(OCCN3CC(CF)C3)cc2)c(F)c1]

### Cis-3-(2-fluoro-4-methoxyphenyl)-4-(4-(2-(3-(fluoromethyl)azetidin-1-yl)ethoxy)phenyl)-7-hydroxythiochroman

### Synthetic Route:

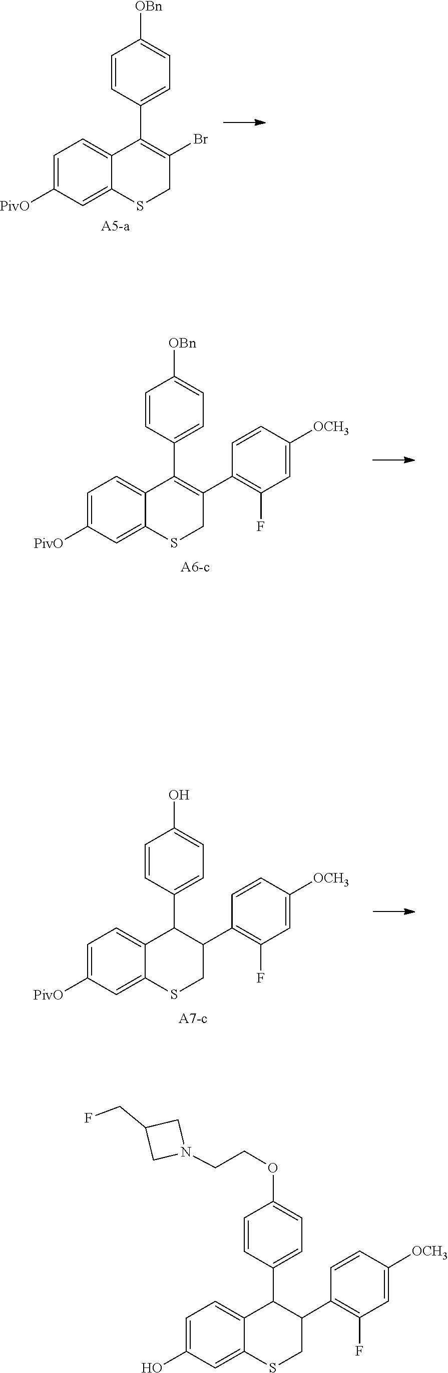

### Step 1

A5-a (0.9 g, 1.77 mmol), 2-fluoro-4-methoxyphenylboronic acid (0.36 g, 2.12 mmol), Pd(PPh₃)₄ (204 mg, 0.17 mmol) and cesium carbonate (1.2 g, 3.54 mmol) were added to a reaction flask, and a mixture of 1,4-dioxane-water (4:1 v/v, 20 mL) was added, and the mixture was reacted in an oil bath at 80° C. for 4 hours under nitrogen protection. After the reaction solution was cooled, it was diluted with water (50 mL), extracted with ethyl acetate (50 mL×2), the organic phases were combined, washed with saturated sodium chloride solution (40 mL), dried over anhydrous sodium sulfate, filtered, concentrated under reduced pressure to remove the solvent, and separated by column chromatography (petroleum ether/ethyl acetate/dichloromethane 15:1) to obtain a white solid A6-c (0.57 g, 58%). ¹H NMR (400 MHz, CDCl₃) δ 7.47-7.33 (m, 5H), 7.16 (d, J=2.4 Hz, 1H), 6.98 (d, J=8.6 Hz, 2H), 6.92 (d, J=8.6 Hz, 1H), 6.86-6.79 (m, 3H), 6.74 (dd, J=8.6, 2.4 Hz, 1H), 6.57 (dd, J=12.0, 2.4 Hz, 1H), 6.42 (dd, J=8.6, 2.4 Hz, 1H), 5.03 (s, 2H), 3.76 (s, 3H), 3.68 (s, 2H), 1.39 (s, 9H).

### Step 2

A6-c (0.55 g) was dissolved in ethanol (15 mL), and 20% Pd(OH)₂/C (0.5 g) was added, and the mixture was reacted under a hydrogen atmosphere (1 atm) at 60° C. for 24 h. The mixture was filtered through diatomaceous earth and concentrated under reduced pressure to remove the solvent to obtain a crude product, which was then separated by column chromatography (petroleum ether/ethyl acetate 3:1) to obtain a white solid A7-c (0.31 g, 67%). ¹H NMR (400 MHz, CDCl₃) δ 7.01-6.95 (m, 2H), 6.72-6.64 (m, 2H), 6.60-6.55 (m, 2H), 6.52-6.44 (m, 3H), 6.24 (t, J=8.6 Hz, 1H), 5.01 (s, 1H), 4.25 (d, J=3.3 Hz, 1H), 3.86 (ddd, J=12.8, 2.9, 2.9 Hz, 1H), 3.81 (s, 3H), 3.41 (t, J=12.4 Hz, 1H), 2.79-2.71 (m, 1H), 1.37 (s, 9H).

### Step 3

A7-c (0.30 g, 0.49 mmol) was dissolved in acetonitrile (15 mL), and 1,2-dibromoethane (2 mL) and potassium carbonate (340 mg, 2.4 mmol) were added. After the reactants were refluxed in an oil bath for 12 h, the insoluble matter was removed by filtration, and the solvent was removed by concentration under reduced pressure. The crude product was used directly in the next step without purification.

The crude product obtained in the previous step was dissolved in acetonitrile (15 mL), 3-fluoromethyl-azetidine hydrochloride (100 mg, 0.78 mmol) and potassium carbonate (340 mg, 2.4 mmol) were added, and after refluxing in an oil bath for 4 h, the insoluble matter was filtered off, and the solvent was removed by concentration under reduced pressure. The crude product was dissolved in methanol (5 mL), sodium methoxide (0.5 mL) was added, and the mixture was stirred at room temperature for 10 min, and then concentrated under reduced pressure to remove the solvent. The mixture was neutralized with 1N HCl to pH 7-8, and extracted with dichloromethane (15 mL×3). The organic phases were combined, and then dried over anhydrous sodium sulfate, filtered, and concentrated under reduced pressure to remove the solvent to obtain a crude product. The crude product was separated by column chromatography (dichloromethane/methanol 30:1-10:1) to obtain Example 3 (71 mg, the total yield of the three steps was 22%). ¹H NMR (500 MHz, CDCl₃) δ 6.74 (d, J=8.3 Hz, 1H), 6.67 (d, J=2.4 Hz, 1H), 6.64 (dd, J=12.0, 2.5 Hz, 1H), 6.51 (d, J=8.4 Hz, 2H), 6.48-6.38 (m, 4H), 6.19 (t, J=8.6 Hz, 1H), 4.49 (dd, J=47.4, 5.3 Hz, 2H), 4.17 (d, J=3.4 Hz, 1H), 3.95-3.80 (m, 3H), 3.78 (s, 3H), 3.58 (t, J=8.0, 8.0 Hz, 1H), 3.35 (t, J=12.4 Hz, 1H), 3.25 (q, J=6.8 Hz, 2H), 2.97-2.82 (m, 3H), 2.66 (d, J=11.6 Hz, 1H).

### Example 4

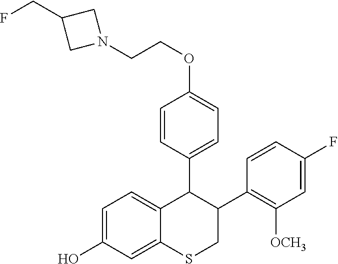 [SMILES: COc1cc(F)ccc1C1CSc2cc(O)ccc2C1c1ccc(OCCN2CC(CF)C2)cc1]

### Cis-3-(4-fluoro-2-methoxyphenyl)-4-(4-(2-(3-(fluoromethyl)azetidin-1-yl)ethoxy)phenyl)-7-hydroxythiochroman

### Synthetic Route:

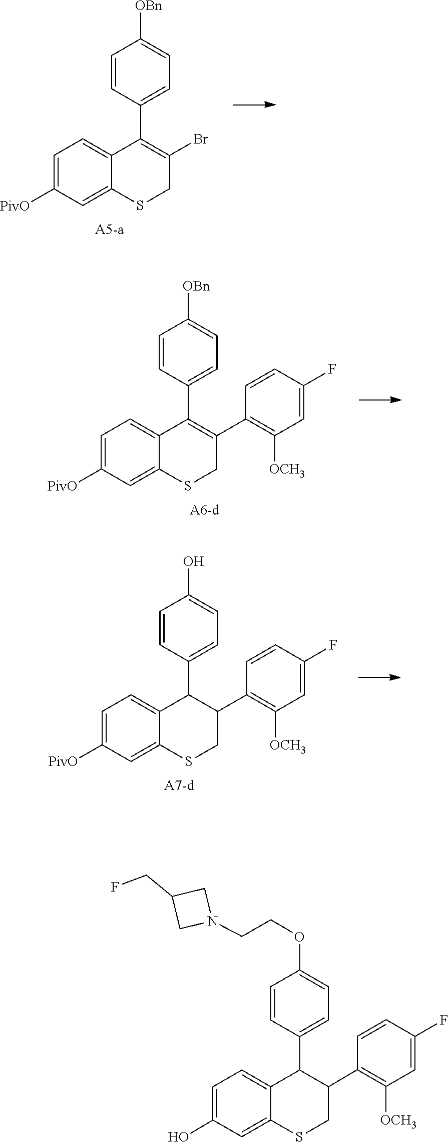

### Step 1

A5-a (0.62 g, 1.2 mmol), 4-fluoro-2-methylphenylboronic acid (0.31 g, 1.8 mmol), Pd(PPh₃)₄ (138 mg, 0.12 mmol) and cesium carbonate (0.8 g, 2.4 mmol) were added to a reaction flask, and a mixture of 1,4-dioxane-water (4:1 v/v, 15 mL) was added, and the mixture was reacted in an oil bath at 80° C. for 4 hours under nitrogen protection. After the reaction solution was cooled, it was diluted with water (50 mL), extracted with ethyl acetate (50 mL×2), the organic phases were combined, washed with saturated sodium chloride solution (40 mL), dried over anhydrous sodium sulfate, filtered, concentrated under reduced pressure to remove the solvent, and separated by column chromatography (petroleum ether/ethyl acetate/dichloromethane 10:1) to obtain a white solid A6-d (0.54 g, 80%). ¹H NMR (400 MHz, CDCl₃) δ 7.46-7.33 (m, 5H), 7.14 (d, J=2.5 Hz, 1H), 6.95-6.88 (m, 3H), 6.83-6.76 (m, 3H), 6.76-6.70 (m, 1H), 6.57 (dd, J=10.8, 2.5 Hz, 1H), 6.37 (td, J=8.4, 2.4 Hz, 1H), 5.02 (s, 2H), 4.00 (br, 1H), 3.82 (s, 3H), 3.25 (br, 1H), 1.38 (s, 9H).

### Step 2

A6-d (0.52 g) was dissolved in a mixed solvent of ethanol/ethyl acetate (1:1 v/v, 15 mL), and 20% Pd(OH)₂/C (0.4 g) was added. The mixture was reacted under a hydrogen atmosphere (1 atm) at 60° C. for 8 h. The mixture was filtered through diatomaceous earth and concentrated under reduced pressure to remove the solvent, and the crude product was separated by column chromatography (petroleum ether/ethyl acetate 3:1) to obtain a white solid A7-d (0.37 g, 86%). ¹H NMR (400 MHz, CDCl₃) δ 7.00-6.95 (m, 2H), 6.71-6.64 (m, 2H), 6.55 (d, J=8.5 Hz, 2H), 6.41 (d, J=8.5 Hz, 3H), 6.16 (dd, J=8.5, 6.6 Hz, 1H), 5.54 (s, 1H), 4.28 (d, J=3.3 Hz, 1H), 3.93 (ddd, J=12.9, 2.8, 2.8 Hz, 1H), 3.87 (s, 3H), 3.38 (dd, J=12.5, 12.5 Hz, 1H), 2.69 (d, J=11.1 Hz, 1H), 1.38 (s, 9H).

### Step 3

A7-d (0.18 g, 0.39 mmol) was dissolved in acetonitrile (10 mL), and 1,2-dibromoethane (2 mL) and potassium carbonate (340 mg, 2.4 mmol) were added. After the reactants were refluxed in an oil bath for 12 h, the insoluble matter was removed by filtration, and the solvent was removed by concentration under reduced pressure. The crude product was used directly in the next step without purification.

The crude product obtained in the previous step was dissolved in acetonitrile (15 mL), 3-fluoromethyl-azetidine hydrochloride (65 mg, 0.52 mmol) and potassium carbonate (269 mg, 1.95 mmol) were added, and after refluxing in an oil bath for 4 h, the insoluble matter was filtered off, and the solvent was removed by concentration under reduced pressure. The crude product was dissolved in methanol (5 mL), sodium methoxide (0.5 mL) was added, and the mixture was stirred at room temperature for 10 min, and then concentrated under reduced pressure to remove the solvent. The mixture was neutralized with 1N HCl to pH 7-8, and extracted with dichloromethane (15 mL×3). The organic phases were combined, and then dried over anhydrous sodium sulfate, filtered, and concentrated under reduced pressure to remove the solvent to obtain a crude product, which was then separated by column chromatography (dichloromethane/methanol 30:1-10:1) to obtain Example 4 (85 mg, total yield of three steps was 45%). ¹H NMR (600 MHz, CDCl₃) δ 6.75 (d, J=8.4 Hz, 1H), 6.66 (d, J=2.4 Hz, 1H), 6.63 (dd, J=10.9, 2.4 Hz, 1H), 6.47 (d, J=8.7 Hz, 2H), 6.42 (dd, J=8.3, 2.5 Hz, 1H), 6.41-6.34 (m, 3H), 6.13-6.07 (m, 1H), 4.49 (dd, J=47.4, 5.3 Hz, 2H), 4.19 (d, J=3.7 Hz, 1H), 3.95-3.80 (m, 6H), 3.60-3.53 (m, 2H), 3.31 (dd, J=12.4, 12.4 Hz, 1H), 3.26-3.20 (m, 2H), 2.96-2.82 (m, 3H), 2.58 (d, J=11.2 Hz, 1H).

### Example 5

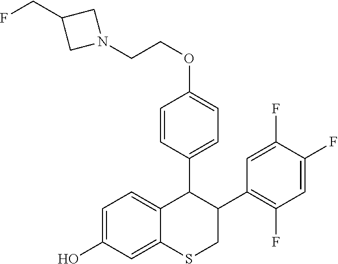 [SMILES: Oc1ccc2c(c1)SCC(c1cc(F)c(F)cc1F)C2c1ccc(OCCN2CC(CF)C2)cc1]

### Cis-4-(4-(2-(3-(fluoromethyl)azetidin-1-yl)ethoxy)phenyl)-3-(2,4,5-trifluorophenyl)-7-hydroxythiochroman

### Synthetic Route:

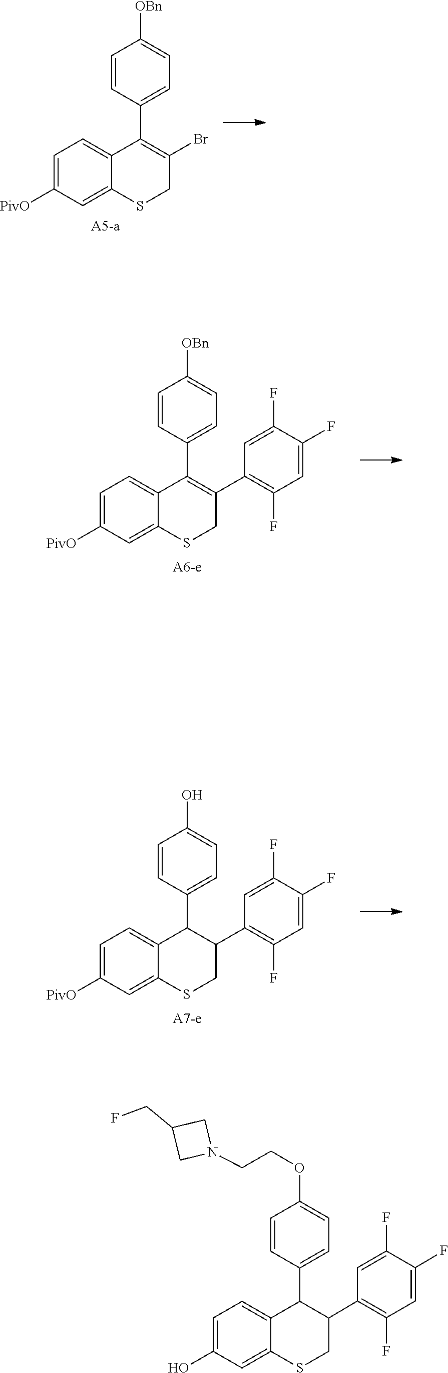

### Step 1

A5-a (0.9 g, 1.77 mmol), 2,4-5-trifluorophenylboronic acid (0.37 g, 2.12 mmol), Pd(PPh₃)₄ (204 mg, 0.17 mmol) and cesium carbonate (1.2 g, 3.54 mmol) were added to a reaction flask, and a mixture of 1,4-dioxane-water (4:1 v/v, 20 mL) was added, and the mixture was reacted in an oil bath at 80° C. for 4 hours under nitrogen protection. After the reaction solution was cooled, it was diluted with water (50 mL), extracted with ethyl acetate (50 mL×2), the organic phases were combined, washed with saturated sodium chloride solution (40 mL), dried over anhydrous sodium sulfate, filtered, concentrated under reduced pressure to remove the solvent, and separated by column chromatography (petroleum ether/ethyl acetate/dichloromethane 15:1) to obtain a white solid A6-e (0.83 g, 84%). ¹H NMR (400 MHz, CDCl₃) δ 7.48-7.34 (m, 5H), 7.17 (d, J=2.4 Hz, 1H), 6.98-6.84 (m, 6H), 6.80-6.71 (m, 2H), 5.06 (s, 2H), 3.66 (s, 2H), 1.39 (s, 9H).

### Step 2

A6-e (0.8 g) was dissolved in ethanol (15 mL), and 20% Pd(OH)₂/C (0.8 g) was added, and the mixture was reacted under a hydrogen atmosphere (1 atm) at 60° C. for 24 h. The mixture was filtered through diatomaceous earth and concentrated under reduced pressure to remove the solvent, and then the obtained crude product was separated by column chromatography (petroleum ether/ethyl acetate 3:1) to obtain a white solid A7-e (0.45 g, 67%). ¹H NMR (400 MHz, CDCl₃) δ 7.04-6.92 (m, 3H), 6.71 (dd, J=8.4, 2.4 Hz, 1H), 6.62 (d, J=8.5 Hz, 2H), 6.49 (d, J=8.5 Hz, 2H), 6.20-6.10 (m, 1H), 5.11 (s, 1H), 4.25 (d, J=3.4 Hz, 1H), 3.93-3.84 (m, 1H), 3.37 (dd, J=12.4, 12.4 Hz, 1H), 2.77-2.68 (m, 1H), 1.37 (s, 9H).

### Step 3

A7-e (0.40 g, 0.85 mmol) was dissolved in acetonitrile (15 mL), and 1,2-dibromoethane (2 mL) and potassium carbonate (280 mg, 4.2 mmol) were added. After the reactants were refluxed in an oil bath for 12 h, the insoluble matter was removed by filtration, and the solvent was removed by concentration under reduced pressure. After the residue was separated by column chromatography (petroleum ether/ethyl acetate 10:1), 270 mg of a colorless oil was obtained.

The crude product obtained in the previous step was dissolved in acetonitrile (15 mL), 3-fluoromethyl-azetidine hydrochloride (180 mg, 1.44 mmol) and potassium carbonate (550 mg, 4.0 mmol) were added, and after refluxing in an oil bath for 4 h, the insoluble matter was filtered off, and the solvent was removed by concentration under reduced pressure. The crude product was dissolved in methanol (5 mL), sodium methoxide (0.5 mL) was added, and the mixture was stirred at room temperature for 10 min, and then concentrated under reduced pressure to remove the solvent. The mixture was neutralized with 1N HCl to pH 7-8, and extracted with dichloromethane (15 mL×3). The organic phases were combined, and then dried over anhydrous sodium sulfate, filtered, and concentrated under reduced pressure to remove the solvent to obtain a crude product. The crude product was separated by column chromatography (dichloromethane/methanol 30:1-10:1) to obtain Example 5 (158 mg, total yield of 2 steps was 68%). ¹H NMR (500 MHz, CDCl₃) δ 6.99-6.92 (m, 1H), 6.74 (d, J=8.4 Hz, 1H), 6.67 (d, J=2.4 Hz, 1H), 6.52 (d, J=8.7 Hz, 2H), 6.48-6.41 (m, 3H), 6.15-6.05 (m, 1H), 4.50 (dd, J=47.4, 5.2 Hz, 2H), 4.17 (d, J=3.5 Hz, 1H), 3.95-3.83 (m, 3H), 3.58 (t, J=7.8 Hz, 2H), 3.35-3.22 (m, 3H), 2.99-2.83 (m, 3H), 2.63 (d, J=11.1 Hz, 1H).

### Example 6

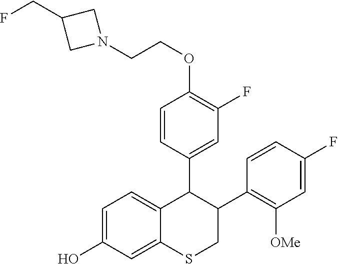 [SMILES: COc1cc(F)ccc1C1CSc2cc(O)ccc2C1c1ccc(OCCN2CC(CF)C2)c(F)c1]

### Cis-3-(4-fluoro-2-methoxyphenyl)-4-(3-fluoro-4-(2-(3-(fluoromethyl)azetidin-1-yl)ethoxy)phenyl)-7-hydroxythiochroman

### Synthetic Route:

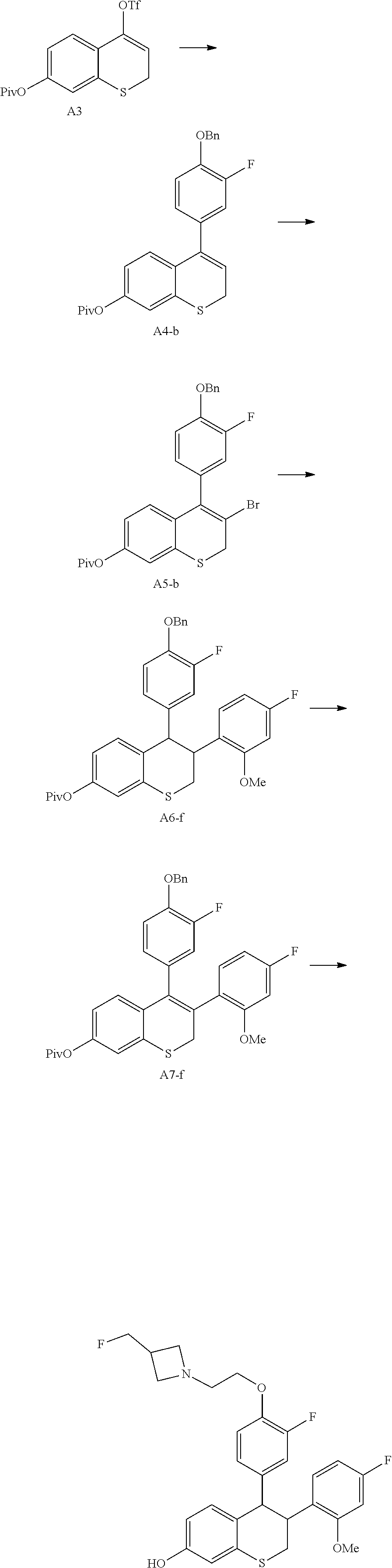

### Step 1

A3 (1.0 g, 2.53 mmol), 3-fluoro-4-benzyloxyphenylboronic acid (0.6 g, 2.42 mmol), Pd(dppf)Cl₂ (0.25 g, 0.31 mmol) and cesium carbonate (1.8 g, 5.5 mmol) were added into a reaction bottle, 1,4-dioxane-water mixture (4:1 v/v, 100 mL) was added, and the mixture was reacted in an oil bath at 50° C. for 1 hour under nitrogen protection. Water (50 mL) was added for dilution, and the mixture was extracted with ethyl acetate (50 mL×3). The organic phases were combined, washed with saturated sodium chloride solution (100 mL), dried over anhydrous sodium sulfate, filtered, and concentrated under reduced pressure to remove the solvent. A4-b (0.97 g, 89%) was obtained as a white solid by column chromatography (petroleum ether/ethyl acetate 10:1). ¹H NMR (400 MHz, CDCl₃) δ 7.52-7.33 (m, 5H), 7.16-6.95 (m, 5H), 6.79 (dd, J=8.5, 2.5 Hz, 1H), 6.02 (t, J=5.7 Hz, 1H), 5.19 (s, 2H), 3.44 (d, J=5.8 Hz, 2H), 1.38 (s, 9H).

### Step 2

A4-b (0.95 g, 2.12 mmol) was dissolved in dichloromethane (100 mL), cooled in an ice-water bath, and tribromopyridinium salt (0.83 g, 2.12 mmol, 85% content) was added. After stirring in an ice-water bath for 1 hour, the mixture was quenched with a saturated sodium bicarbonate solution (50 mL), separated, the aqueous phase was extracted with dichloromethane (50 mL), the organic phases were combined, washed with a saturated sodium chloride solution (50 mL), dried over anhydrous sodium sulfate, filtered, and concentrated under reduced pressure to remove the solvent to obtain a crude product, which was separated by column chromatography (petroleum ether/ethyl acetate 10:1) to obtain a light yellow solid A5-b (0.85 g, 76%). ¹H NMR (400 MHz, CDCl3) δ 7.53-7.48 (m, 2H), 7.47-7.41 (m, 2H), 7.41-7.35 (m, 1H), 7.11-7.05 (m, 2H), 7.00 (dd, J=11.7, 2.1 Hz, 1H), 6.93-6.88 (m, 1H), 6.72 (s, 2H), 5.21 (s, 2H), 3.95 (s, 2H), 1.36 (s, 9H).

### Step 3

A5-b (0.85 g, 1.62 mmol), 4-fluoro-2-methoxyphenylboronic acid (0.33 g, 1.94 mmol), Pd(PPh₃)₄ (128 mg, 0.16 mmol) and cesium carbonate (1.0 g, 3.14 mmol) were added to a reaction flask, and a mixture of 1,4-dioxane-water (4:1 v/v, 20 mL) was added, and the mixture was reacted in an oil bath at 80° C. for 4 hours under nitrogen protection. After the reaction solution was cooled, it was diluted with water (50 mL), extracted with ethyl acetate (50 mL×2), the organic phases were combined, washed with saturated sodium chloride solution (40 mL), dried over anhydrous sodium sulfate, filtered, concentrated under reduced pressure to remove the solvent, and separated by column chromatography (petroleum ether/ethyl acetate/dichloromethane 15:1) to obtain a white solid A6-f (0.71 g, 79%). ¹H NMR (400 MHz, CDCl₃) δ 7.45-7.35 (m, 5H), 7.14 (d, J=2.4 Hz, 1H), 6.87 (d, J=8.6 Hz, 1H), 6.83-6.72 (m, 4H), 6.69-6.64 (m, 1H), 6.56 (dd, J=10.8, 2.4 Hz, 1H), 6.38 (td, J=8.3, 2.4 Hz, 1H), 5.10 (s, 2H), 3.81 (s, 3H), 1.38 (s, 9H).

### Step 4

A6-a (0.32 g) was dissolved in ethanol (15 mL), and 20% Pd(OH)₂/C (0.35 g) was added, and the mixture was reacted under a hydrogen atmosphere (1 atm) at 60° C. for 8 h. The crude product was filtered through diatomaceous earth and concentrated under reduced pressure to remove the solvent, and then separated by column chromatography (petroleum ether/ethyl acetate 3:1) to obtain a white solid A7-f (0.17 g, 63%). ¹H NMR (400 MHz, CDCl₃) δ 7.02-6.94 (m, 2H), 6.76-6.65 (m, 3H), 6.45 (td, J=8.3, 2.5 Hz, 1H), 6.31 (dd, J=11.7, 1.8 Hz, 1H), 6.25-6.17 (m, 2H), 5.22 (s, 1H), 4.28 (d, J=3.7 Hz, 1H), 3.99-3.91 (m, 1H), 3.88 (s, 3H), 3.37 (dd, J=12.5, 12.5 Hz, 1H), 2.72 (d, J=12.0 Hz, 1H), 1.38 (s, 9H).

### Step 5

A7-a (165 mg, 0.55 mmol) was dissolved in acetonitrile (15 mL), 1,2-dibromoethane (2 mL) and potassium carbonate (380 mg, 2.75 mmol) were added, and refluxed in an oil bath for 12 h. The insoluble matter was filtered out and the solvent was removed by concentration under reduced pressure. The crude product was used directly in the next step without purification.

The crude product obtained in the previous step was dissolved in acetonitrile (15 mL), and 3-fluoromethyl-azetidine hydrochloride (103 mg, 0.83 mmol) and potassium carbonate (380 mg, 2.75 mmol) were added. After refluxing in an oil bath for 4 h, the insoluble matter was filtered out and the solvent was removed by concentration under reduced pressure. The crude product was dissolved in methanol (5 mL), sodium methoxide (0.5 mL) was added, and the mixture was stirred at room temperature for 10 min, then concentrated under reduced pressure to remove the solvent, neutralized with 1N HCl to pH 7-8, and extracted with dichloromethane (15 mL×3). The organic phases were combined, then dried over anhydrous sodium sulfate, filtered, and concentrated under reduced pressure to remove the solvent to obtain a crude product, which was separated by column chromatography (dichloromethane/methanol 30:1-10:1) to obtain Example 6 (98 mg, total yield of three steps was 56%). ¹H NMR (500 MHz, CDCl₃) δ 6.74 (d, J=8.3 Hz, 1H), 6.70-6.58 (m, 3H), 6.49-6.39 (m, 2H), 6.29-6.14 (m, 3H), 4.52 (dd, J=47.4, 5.1 Hz, 2H), 4.20 (s, 1H), 4.06-3.81 (m, 6H), 3.68-3.55 (m, 2H), 3.36-3.27 (m, 3H), 3.01-2.86 (m, 3H), 2.64 (d, J=11.6 Hz, 1H).

### Example 7

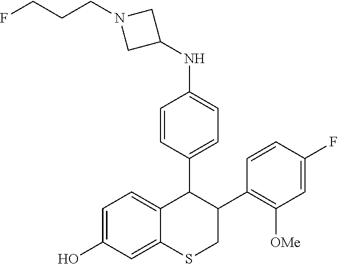 [SMILES: COc1cc(F)ccc1C1CSc2cc(O)ccc2C1c1ccc(NC2CN(CCCF)C2)cc1]

### Cis-3-(4-fluoro-2-methoxyphenyl)-4-(4-(1-(3-fluoropropyl)azetidin-3-yl)amino)phenyl)-7-hydroxythiochroman

### Synthetic Route:

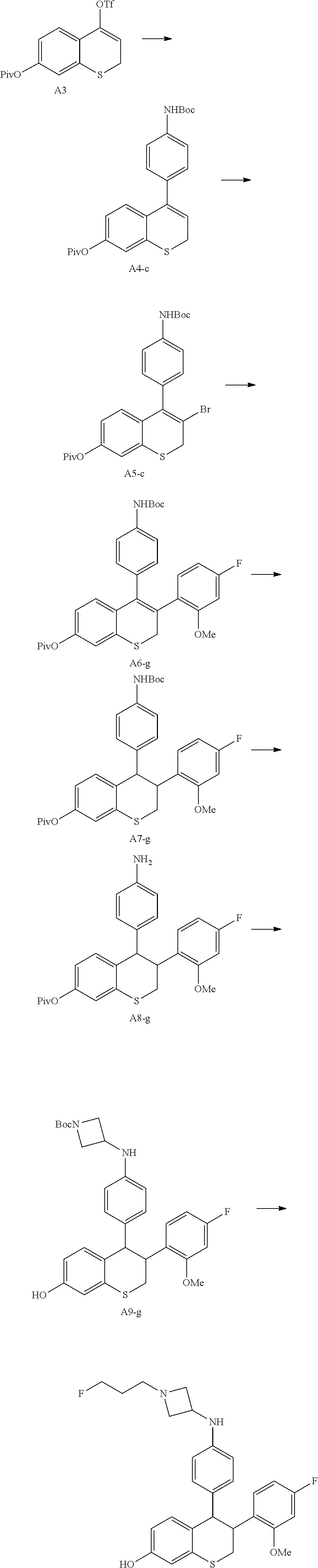

### Step 1

A3 (3.0 g, 7.6 mmol), 4-(tert-butoxycarbonylamino)-phenylboronic acid (1.94 g, 9.1 mmol), Pd(dppf)Cl₂ (0.62 g, 0.76 mmol) and cesium carbonate (4.9 g, 14.2 mmol) were added into a reaction bottle, 1,4-dioxane-water mixture (4:1 v/v, 30 mL) was added, and the mixture was reacted in an oil bath at 50° C. for 1 hour under nitrogen protection. Water (50 mL) was added for dilution, and ethyl acetate (50 mL×3) was used for extraction. The organic phases were combined, washed with saturated sodium chloride solution (100 mL), dried over anhydrous sodium sulfate, filtered, and concentrated under reduced pressure to remove the solvent. A4-c (3.1 g, 93%) was obtained as a white solid by column chromatography separation (petroleum ether/ethyl acetate 10:1). ¹H NMR (400 MHz, CDCl₃) δ7.36 (d, J=8.5 Hz, 2H), 7.21 (d, J=8.5 Hz, 2H), 7.11 (d, J=2.4 Hz, 1H), 7.04 (d, J=8.5 Hz, 1H), 6.75 (dd, J=8.5, 2.4 Hz, 1H), 6.60 (d, J=6.1 Hz, 1H), 6.01 (t, J=5.7 Hz, 1H), 3.44 (d, J=5.7 Hz, 2H), 1.55 (s, 9H), 1.37 (s, 9H).

### Step 2

A4-b (2.8 g, 6.4 mmol) was dissolved in dichloromethane (100 mL), pyridine (1.0 g, 12.8 mmol) was added, and the mixture was cooled in an ice-water bath, and tribromopyridinium salt (2.5 g, 6.4 mmol, 85% content) was added. After stirring in an ice-water bath for 1 hour, the mixture was quenched with a saturated sodium bicarbonate solution (100 mL), and separated. The aqueous phase was extracted with dichloromethane (50 mL), and the organic phases were combined, washed with a saturated sodium chloride solution (50 mL), dried over anhydrous sodium sulfate, filtered, and concentrated under reduced pressure to remove the solvent to obtain a crude product, which was separated by column chromatography (petroleum ether/ethyl acetate 10:1) to obtain a light yellow foam solid A5-c (2.0 g, 60%). ¹H NMR (400 MHz, CDCl₃) δ7.43 (d, J=8.5 Hz, 2H), 7.13 (d, J=8.6 Hz, 2H), 7.04 (d, J=2.3 Hz, 1H), 6.74-6.63 (m, 3H), 3.95 (s, 2H), 1.55 (s, 9H), 1.35 (s, 9H).

### Step 3

A5-c (1.0 g, 1.93 mmol), 4-fluoro-2-methoxyphenylboronic acid (0.39 g, 2.31 mmol), Pd(PPh₃)₄ (210 mg, 0.19 mmol) and cesium carbonate (1.3 g, 3.8 mmol) were added to a reaction flask, and a mixture of 1,4-dioxane-water (4:1 v/v, 20 mL) was added, and the mixture was reacted in an oil bath at 80° C. for 4 hours under nitrogen protection. After the reaction solution was cooled, it was diluted with water (50 mL), extracted with ethyl acetate (50 mL×2), the organic phases were combined, washed with saturated sodium chloride solution (40 mL), dried over anhydrous sodium sulfate, filtered, and concentrated under reduced pressure to remove the solvent, and separated by column chromatography (petroleum ether/ethyl acetate 10:1) to obtain a white solid A6-g (0.91 g, 84%). ¹H NMR (400 MHz, CDCl₃) δ 7.18 (d, J=7.9 Hz, 2H), 7.13 (d, J=2.4 Hz, 1H), 6.92 (d, J=8.0 Hz, 2H), 6.88 (d, J=8.6 Hz, 1H), 6.79 (dd, J=8.4, 6.9 Hz, 1H), 6.70 (dd, J=8.6, 2.4 Hz, 1H), 6.55 (dd, J=10.8, 2.4 Hz, 1H), 6.51 (s, 1H), 6.35 (td, J=8.4, 2.4 Hz, 1H), 3.99 (br, 1H), 3.81 (s, 3H), 3.25 (br, 1H), 1.52 (s, 9H), 1.37 (s, 9H).

### Step 4

A6-g (0.9 g) was dissolved in ethanol (15 mL), and 20% Pd(OH)₂/C (0.85 g) was added, and the mixture was reacted under a hydrogen atmosphere (1 atm) at 60° C. for 8 h. The crude product was filtered through diatomaceous earth and concentrated under reduced pressure to remove the solvent, and the crude product was separated by column chromatography (petroleum ether/ethyl acetate 5:1) to obtain a white solid A7-g (0.8 g, 89%). ¹H NMR (400 MHz, CDCl₃) δ 7.09 (d, J=8.2 Hz, 2H), 7.00-6.93 (m, 2H), 6.71-6.64 (m, 2H), 6.51-6.46 (m, 3H), 6.42 (td, J=8.3, 2.4 Hz, 1H), 6.17 (dd, J=8.4, 6.8 Hz, 1H), 4.30 (d, J=3.3 Hz, 1H), 3.95 (dt, J=12.9, 2.7 Hz, 1H), 3.87 (s, 2H), 3.38 (t, J=12.5 Hz, 1H), 2.70 (d, J=11.1 Hz, 3H), 1.51 (s, 9H), 1.38 (s, 9H).

### Step 5

A7-g (0.76 g) was dissolved in 1,4-dioxane (10 mL), and hydrogen chloride (4M 1,4-dioxane solution, 4 mL) was added, and the mixture was reacted at room temperature for 2 h. The solvent was removed by concentration under reduced pressure to obtain a crude product, which was then dissolved in dichloromethane (20 mL), then washed with a saturated sodium bicarbonate solution, separated, and the organic phase was dried over anhydrous sodium sulfate, filtered, and concentrated to obtain a crude product, which was separated by column chromatography (petroleum ether/ethyl acetate 5:1) to obtain a white solid A8-g (0.58 g, 93%). ¹H NMR (400 MHz, CDCl₃) δ 7.02-6.93 (m, 2H), 6.71-6.63 (m, 2H), 6.46-6.40 (m, 3H), 6.34 (d, J=8.3 Hz, 2H), 6.20 (dd, J=8.5, 6.7 Hz, 1H), 4.23 (d, J=3.2 Hz, 1H), 3.95-3.90 (m, 1H), 3.87 (s, 3H), 3.56 (br, 2H), 3.40 (dd, J=12.4, 12.4 Hz, 1H), 2.68 (d, J=11.8 Hz, 1H), 1.37 (s, 9H).

### Step 6

A8-g (0.56 g, 1.2 mmol) and N-tert-butyloxycarbonyl-3-azetidinone (0.25 g, 1.44 mmol) were dissolved in 1,2-dichloroethane (10 mL), 2 drops of acetic acid were added, and the mixture was stirred at room temperature for 10 minutes. Sodium triacetoxyborohydride (0.76 g, 3.6 mmol) was added, and the mixture was reacted at room temperature for 12 h. Saturated sodium bicarbonate solution (10 mL) was added, and the mixture was stirred for 10 minutes. The mixture was extracted with dichloromethane (20 mL×2), and the organic phase was dried over anhydrous sodium sulfate, filtered, and concentrated to obtain a crude product, which was separated by column chromatography (petroleum ether/ethyl acetate 5:1) to obtain a white solid A9-g (0.56 g, 76%). ¹H NMR (400 MHz, CDCl₃) δ 6.99-6.91 (m, 2H), 6.69-6.61 (m, 2H), 6.43-6.32 (m, 3H), 6.23 (d, J=8.2 Hz, 2H), 6.15 (dd, J=8.4, 6.5 Hz, 1H), 4.26-4.18 (m, 3H), 4.10 (d, J=6.4 Hz, 1H), 3.85 (s, 4H), 3.72-3.64 (m, 2H), 3.36 (dd, J=12.4, 12.4 Hz, 1H), 2.66 (d, J=11.0 Hz, 1H), 1.43 (s, 9H), 1.35 (s, 9H).

### Step 7

A9-g (340 mg, 0.54 mmol) was dissolved in 1,4-dioxane (5 mL), and hydrogen chloride (4M 1,4-dioxane solution, 2 mL) was added, and the mixture was reacted at room temperature for 2 h. The solvent was removed by concentration under reduced pressure to obtain a crude product, which was then dissolved in dichloromethane (20 mL), then washed with a saturated sodium bicarbonate solution, separated, and the organic phase was dried over anhydrous sodium sulfate, filtered, and concentrated to obtain a crude product. The crude product was dissolved in DMF (5 mL), and DIPEA (140 mg, 1.09 mmol) and 3-fluoro-1-iodopropane (98 mg, 0.49 mmol) were added, and stirred at room temperature for 24 h. Water (30 mL) was added, and the mixture was extracted with ethyl acetate (20 mL×2). The organic phase was washed with a saturated sodium chloride solution (20 mL), dried over anhydrous sodium sulfate, filtered, and concentrated to obtain a crude product. The obtained crude product was dissolved in methanol (5 mL), sodium methoxide (0.5 mL) was added, and the mixture was stirred at room temperature for 10 min, then concentrated under reduced pressure to remove the solvent, neutralized with 1N HCl to pH 7-8, and extracted with dichloromethane (15 mL×3). The organic phases were combined, then dried over anhydrous sodium sulfate, filtered, and concentrated under reduced pressure to remove the solvent to obtain a crude product, which was separated by column chromatography (dichloromethane/methanol 30:1-10:1) to obtain a colorless oil, which was freeze-dried to obtain Example 7 (98 mg, total yield of three steps was 56%). ¹H NMR (500 MHz, CDCl₃) δ 6.84 (d, J=8.4 Hz, 1H), 6.71-6.63 (m, 2H), 6.48-6.39 (m, 2H), 6.36 (d, J=8.2 Hz, 2H), 6.26 (d, J=8.2 Hz, 2H), 6.18 (dd, J=8.6, 6.7 Hz, 1H), 4.50 (dt, J=47.1, 5.8 Hz, 4.17 (d, J=3.2 Hz, 1H), 4.08 (p, J=6.1 Hz, 1H), 3.96-3.89 (m, 1H), 3.86 (s, 3H), 3.81-3.70 (m, 3H), 3.40 (dd, J=12.4, 12.4 Hz, 1H), 3.04-2.96 (m, 2H), 2.74-2.61 (m, 3H), 1.88-1.75 (m, 2H).

### Example 8

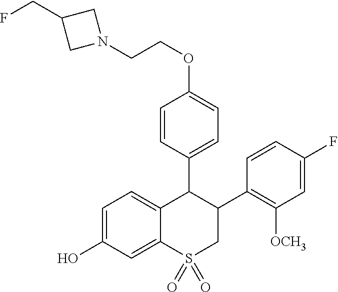 [SMILES: COc1cc(F)ccc1C1CS(=O)(=O)c2cc(O)ccc2C1c1ccc(OCCN2CC(CF)C2)cc1]

### Cis-3-(4-fluoro-2-methoxyphenyl)-4-(4-(2-(3-(fluoromethyl)azetidin-1-yl)ethoxy)phenyl)-7-hydroxythiochroman-1,1-dioxide

### Synthetic Route:

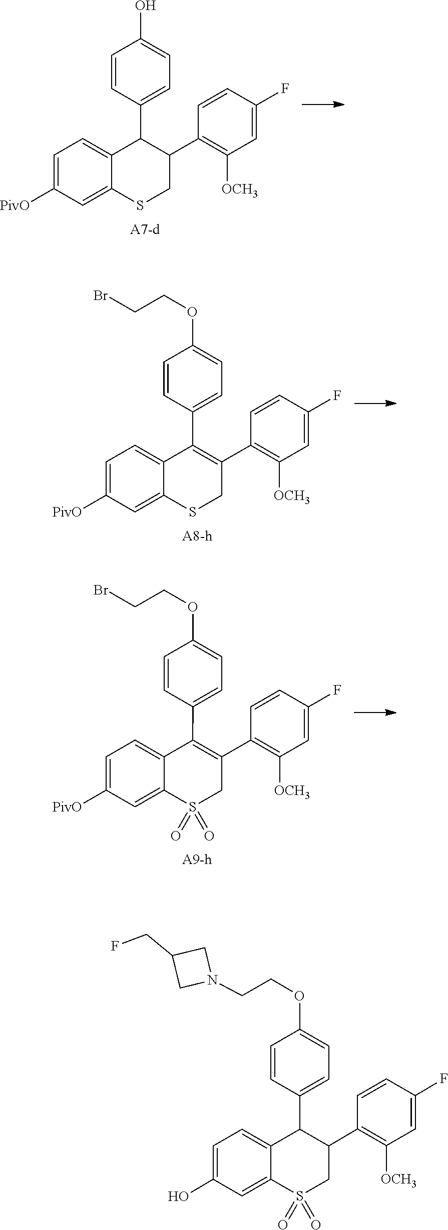

### Step 1

A7-d (0.18 g, 0.39 mmol) was dissolved in acetonitrile (10 mL), and 1,2-dibromoethane (2 mL) and potassium carbonate (340 mg, 2.4 mmol) were added. After the reactants were refluxed in an oil bath for 12 h, the insoluble matter was removed by filtration, and the solvent was removed by concentration under reduced pressure to obtain a crude product A8-h (200 mg) which was directly used in the next step.

### Step 2

The crude product obtained above was dissolved in ether (8 mL), the reaction flask was placed in an ice-water bath, and then m-chloroperbenzoic acid (141 mg, 0.78 mmol) was added and stirred for 6 hours. The reaction was quenched with saturated sodium sulfite solution (5 mL), extracted with dichloromethane, dried over anhydrous sodium sulfate, filtered, and concentrated under reduced pressure to remove the solvent to obtain a crude product, which was separated by column chromatography (petroleum ether/ethyl acetate 5:1) to obtain A9-h (142 mg, total yield of 2 steps was 61%). ¹H NMR (400 MHz, CDCl₃) δ 7.76 (d, J=2.3 Hz, 1H), 7.23 (dd, J=8.5, 2.4 Hz, 1H), 7.16 (d, J=8.6 Hz, 1H), 6.69 (dd, J=10.7, 2.4 Hz, 1H), 6.64 (d, J=8.7 Hz, 2H), 6.44 (td, J=8.3, 2.5 Hz, 1H), 6.36 (d, J=8.6 Hz, 2H), 6.08 (dd, J=8.3, 6.6 Hz, 1H), 4.69-4.59 (m, 1H), 4.56 (d, J=4.1 Hz, 1H), 4.21 (t, J=6.2 Hz, 2H), 3.88 (s, 3H), 3.60 (t, J=6.2 Hz, 2H), 3.27 (d, J=13.5 Hz, 1H), 1.39 (s, 9H).

### Step 3

A9-h (139 mg, 0.23 mmol) was dissolved in acetonitrile (15 mL), and 3-fluoromethyl-azetidine hydrochloride (96 mg, 0.77 mmol) and potassium carbonate (165 mg, 1.2 mmol) were added. After refluxing in an oil bath for 4 h, the insoluble matter was filtered off, and the solvent was removed by concentration under reduced pressure. The crude product was dissolved in methanol (5 mL), sodium methoxide (0.5 mL) was added, and the mixture was stirred at room temperature for 10 min, then concentrated under reduced pressure to remove the solvent, neutralized with 1N HCl to pH 7-8, and extracted with dichloromethane (15 mL×3). The organic phases were combined, then dried over anhydrous sodium sulfate, filtered, and concentrated under reduced pressure to remove the solvent to obtain a crude product, which was separated by column chromatography (dichloromethane/methanol 30:1-10:1) to obtain Example 8 (25 mg, total yield of 2 steps was 21%). ¹H NMR (500 MHz, CDCl₃) δ 7.34 (d, J=2.2 Hz, 1H), 7.28-7.25 (m, 1H), 6.99-6.89 (m, 2H), 6.65 (dd, J=10.8, 2.4 Hz, 1H), 6.45-6.38 (m, 2H), 6.23 (d, J=8.6 Hz, 2H), 6.12-6.06 (m, 1H), 4.60 (dd, J=13.0, 3.6 Hz, 1H), 4.54 (d, J=5.1 Hz, 1H), 4.49-4.40 (m, 2H), 3.94-3.71 (m, 4H), 3.64-3.54 (m, 2H), 3.30-3.22 (m, 2H), 3.16 (d, J=12.9 Hz, 1H), 3.01-2.85 (m, 3H), 2.25-2.19 (m, 1H).

### Example 9

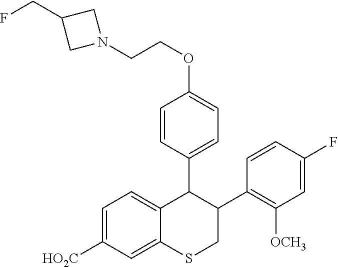 [SMILES: COc1cc(F)ccc1C1CSc2cc(C(=O)O)ccc2C1c1ccc(OCCN2CC(CF)C2)cc1]

### Cis-3-(4-fluoro-2-methoxyphenyl)-4-(4-(2-(3-(fluoromethyl)azetidin-1-yl)ethoxy)phenyl)thiochroman-7-carboxylic Acid

### Synthetic Route:

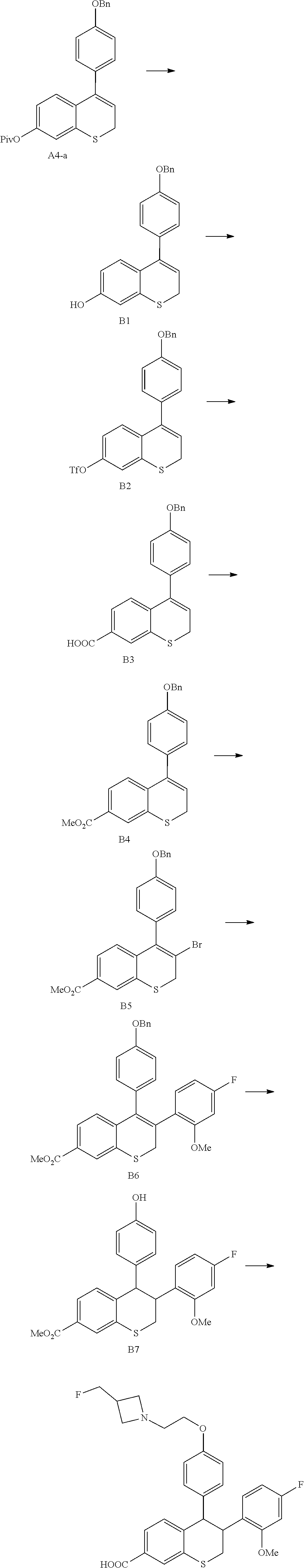

### Step 1

A4-a (2.1 g, 4.9 mmol) was dissolved in methanol (40 mL), and 2M NaOH aqueous solution (5 mL) was added, and stirred at room temperature for 1 h. The pH was adjusted to 7. The mixture was extracted with ethyl acetate (50 mL×2), dried over anhydrous sodium sulfate, filtered, and concentrated under reduced pressure to remove the solvent to obtain a white solid B1 (1.7 g, 100%). LCMS: 347.3 [M+H].

### Step 2

B1 (1.7 g, 4.9 mmol) was dissolved in dichloromethane (50 mL) and triethylamine (1.0 g, 7.4 mmol) was added. The mixture was cooled in an ice-water bath, and tribromopyridinium salt (0.95 mL, 5.4 mmol) was added and stirred in an ice-water bath for 10 min. The mixture was quench with water (100 mL), separated, and the aqueous phase was extracted with dichloromethane (50 mL). The combined organic phases was washed with saturated sodium chloride solution (50 mL), dried over anhydrous sodium sulfate, filtered, and concentrated under reduced pressure to remove the solvent to obtain a crude product, which was separated by column chromatography (petroleum ether/ethyl acetate 10:1) to obtain a white solid B2 (2.3 g, 100%). LCMS: 479.1 [M+H].

### Step 3

Under nitrogen atmosphere, sodium formate (0.75 g, 14.4 mmol), acetic anhydride (0.99 g, 9.6 mmol), DIPEA (1.25 g, 9.6 mmol) and anhydrous DMF (6 mL) were added to a reaction flask, and B2 (2.3 g, 4.8 mmol), Pd (dppf)Cl₂ (0.4 g, 0.49 mmol), LiCl (0.62 g, 14.4 mmol) and anhydrous DMF (20 mL) were added after stirring at room temperature for one hour. The reaction mixture was placed in an 80° C. oil bath for 24 hours. After the reaction solution was cooled, it was diluted with ethyl acetate (80 mL), washed three times with 1N HCl (100 mL), the organic phases were combined, dried over anhydrous sodium sulfate, filtered, and concentrated under reduced pressure to remove the solvent to obtain a crude product. Dichloromethane (10 mL) was added to the obtained crude product, stirred for 5 minutes, and filtered. The obtained filter cake was a light yellow solid B3 (0.72 g), which was directly used in the next step.

### Step 4

The obtained B3 (0.72 g) was dissolved in methanol (20 mL), and 2% concentrated sulfuric acid (0.5 mL) was added, and the mixture was reacted at 65° C. for 8 h. The solvent was removed by concentration under reduced pressure to obtain a crude product, which was then dissolved in dichloromethane (100 mL), and then dissolved in water (50 mL), saturated sodium bicarbonate solution (20 mL) and saturated sodium chloride (50 mL) in sequence. The organic phase was dried over anhydrous sodium sulfate, filtered, and concentrated to obtain a crude product, which was separated by column chromatography (petroleum ether/ethyl acetate 8:1) to obtain B4 (0.78 g, two-step yield 42%). ¹H NMR (400 MHz, CDCl₃) δ 8.07 (d, J=1.8 Hz, 1H), 7.70 (dd, J=8.1, 1.8 Hz, 1H), 7.51-7.34 (m, 5H), 7.23-7.18 (m, 2H), 7.13 (d, J=8.1 Hz, 1H), 7.03-6.98 (m, 2H), 6.15 (t, J=5.8 Hz, 1H), 5.12 (s, 2H), 3.93 (s, 3H), 3.47 (d, J=5.7 Hz, 2H).

### Step 5

B4 (0.75 g, 1.93 mmol) was dissolved in dichloromethane (50 mL), cooled in an ice-water bath, and tribromopyridinium salt (0.77 g, 2.03 mmol) was added. After stirring in an ice-water bath for 1 hour, the mixture was quenched with a saturated sodium bicarbonate solution (100 mL), separated, the aqueous phase was extracted with dichloromethane (50 mL), the organic phases were combined, washed with a saturated sodium chloride solution (50 mL), dried over anhydrous sodium sulfate, filtered, and concentrated under reduced pressure to remove the solvent to obtain a crude product, which was separated by column chromatography (petroleum ether/ethyl acetate 10:1) to obtain B5 (0.50 g, 56%). ¹H NMR (400 MHz, CDCl₃) δ 8.00 (d, J=1.7 Hz, 1H), 7.63 (dd, J=8.3, 1.7 Hz, 1H), 7.53-7.35 (m, 5H), 7.15 (d, J=8.7 Hz, 2H), 7.07 (d, J=8.7 Hz, 2H), 6.81 (d, J=8.3 Hz, 1H), 5.13 (s, 2H), 3.99 (s, 2H), 3.92 (s, 3H).

### Step 6

B5 (0.50 g, 1.07 mmol), 4-fluoro-2-methoxyphenylboronic acid (0.30 g, 1.6 mmol), Pd(PPh₃)₄ (120 mg, 0.1 mmol) and cesium carbonate (0.7 g, 2.1 mmol) were added to a reaction flask, and a mixture of 1,4-dioxane-water (4:1 v/v, 15 mL) was added, and the mixture was reacted in an oil bath at 80° C. for 4 hours under nitrogen protection. After the reaction solution was cooled, it was diluted with water (50 mL), extracted with ethyl acetate (50 mL×2), the organic phases were combined, washed with saturated sodium chloride solution (40 mL), dried over anhydrous sodium sulfate, filtered, concentrated under reduced pressure to remove the solvent, and separated by column chromatography (petroleum ether/ethyl acetate 10:1) to obtain B6 (0.45 g, 82%). ¹H NMR (400 MHz, CDCl₃) δ 8.11 (s, 1H), 7.67 (dd, J=8.2, 1.3 Hz, 1H), 7.49-7.33 (m, 5H), 6.99 (d, J=8.2 Hz, 1H), 6.91 (d, J=8.1 Hz, 2H), 6.85-6.78 (m, 3H), 6.58 (dd, J=10.8, 2.3 Hz, 1H), 6.39 (td, J=8.4, 2.4 Hz, 1H), 5.03 (s, 2H), 4.08-3.87 (m, 4H), 3.82 (s, 3H), 3.31 (br, 1H).

### Step 7

B6 (0.44 g, 0.86 mmol) was dissolved in ethanol (15 mL), and 20% Pd(OH)₂/C (0.5 g) was added. The mixture was reacted under a hydrogen atmosphere (1 atm) at 50° C. for 8 h. The crude product was filtered through diatomaceous earth and concentrated under reduced pressure to remove the solvent. The crude product was separated by column chromatography (petroleum ether/ethyl acetate 5:1) to obtain a white solid B7 (0.26 g, 72%). ¹H NMR (400 MHz, CDCl₃) δ7.99-7.92 (m, 1H), 7.61 (dd, J=8.0, 1.7 Hz, 1H), 7.06 (d, J=8.1 Hz, 1H), 6.68 (dd, J=10.9, 2.4 Hz, 1H), 6.58 (d, J=8.5 Hz, 2H), 6.48-6.36 (m, 3H), 6.18 (dd, J=8.4, 6.8 Hz, 1H), 5.24 (s, 1H), 4.33 (d, J=3.2 Hz, 1H), 3.99-3.90 (m, 4H), 3.88 (s, 3H), 3.42 (dd, J=12.5, 12.5 Hz, 1H), 2.74 (d, J=11.7 Hz, 1H).

### Step 8

B7 (230 mg, 0.54 mmol) was dissolved in acetonitrile (15 mL), and 1,2-dibromoethane (2 mL) and potassium carbonate (380 mg, 2.75 mmol) were added. After the mixture was refluxed in an oil bath for 12 h, the insoluble matter was filtered out and the solvent was removed by concentration under reduced pressure. The crude product was used directly in the next step without purification.

The crude product obtained in the previous step was dissolved in acetonitrile (15 mL), 3-fluoromethyl-azetidine hydrochloride (140 mg, 1.1 mmol) and potassium carbonate (380 mg, 2.75 mmol) were added. After the mixture was refluxed in an oil bath for 4 h, the insoluble matter was filtered off, and the solvent was removed by concentration under reduced pressure. The crude product was dissolved in methanol (5 mL), 2M NaOH (1 mL) was added, and the reaction was carried out at 60° C. for 10 min, and then the solvent was removed by concentration under reduced pressure. The mixture was neutralized to pH 7-8 with 1N HCl, and extracted with dichloromethane (15 mL×3). The organic phases were combined, and then dried over anhydrous sodium sulfate, filtered, and concentrated under reduced pressure to remove the solvent to obtain a crude product, which was separated by column chromatography (dichloromethane/methanol 10:1-8:1) to obtain Example 9 (122 mg, total yield of three steps was 43%). ¹H NMR (500 MHz, CDCl₃) δ7.94 (s, 1H), 7.59 (d, J=7.8 Hz, 1H), 6.97 (d, J=8.0 Hz, 1H), 6.66 (dd, J=10.8, 2.0 Hz, 1H), 6.58 (d, J=8.5 Hz, 2H), 6.42 (dd, J=14.2, 5.3 Hz, 3H), 6.20-6.11 (m, 1H), 5.03 (s, 1H), 4.52 (dd, J=47.3, 3.9 Hz, 2H), 4.32 (d, J=2.9 Hz, 1H), 4.12-3.94 (m, 5H), 3.88 (s, 3H), 3.62 (dt, J=40.7, 8.2 Hz, 2H), 3.43 (dd, J=12.5, 12.5 Hz, 1H), 3.29-3.12 (m, 3H), 2.70 (d, J=11.5 Hz, 1H).

### Example 10

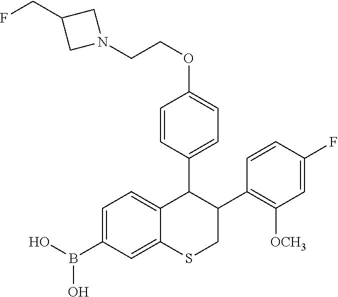 [SMILES: COc1cc(F)ccc1C1CSc2cc(B(O)O)ccc2C1c1ccc(OCCN2CC(CF)C2)cc1]

### Cis-(3-(4-fluoro-2-methoxyphenyl)-4-(4-(2-(3-(fluoromethyl)azetidin-1-yl)ethoxy)phenyl)thiochroman-7-boronic Acid

### Synthetic Route:

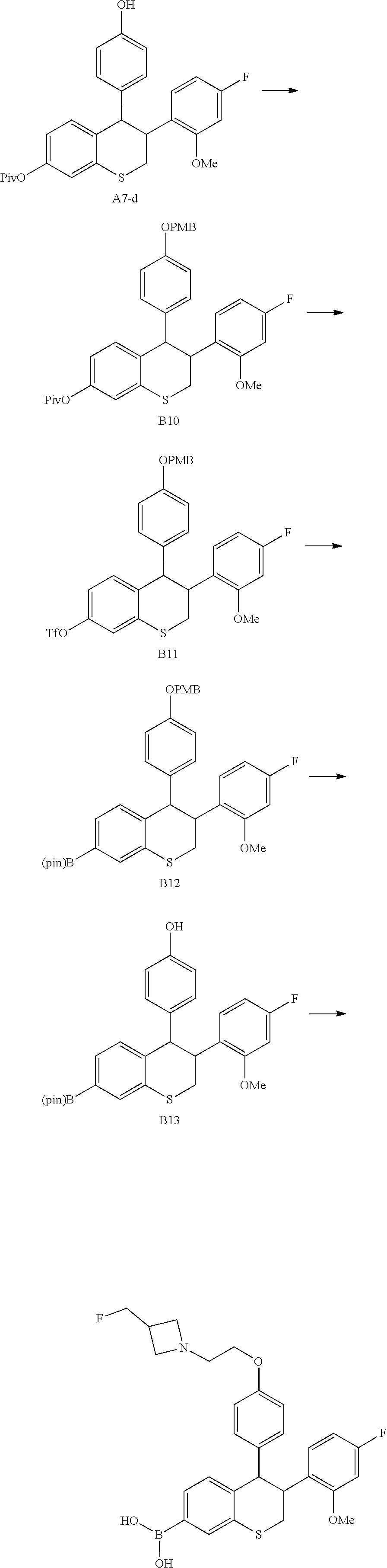

### Step 1

A7-d (1.6 g, 3.4 mmol) and PMBCl (0.64 g, 4.1 mmol) were dissolved in acetone (40 mL), potassium carbonate (0.95 g, 6.8 mmol), KI (57 mg, 0.34 mmol) and tetrabutylammonium bromide (0.1 g) were added, and the mixture was reacted at 60° C. for 12 h. The insoluble matter was removed by filtration, and B10 (1.7 g, 85%) was obtained by column chromatography (petroleum ether/ethyl acetate 5:1). ¹H NMR (400 MHz, CDCl₃) δ 7.33 (d, J=8.4 Hz, 2H), 6.98 (d, J=8.7 Hz, 2H), 6.91 (d, J=8.4 Hz, 2H), 6.73-6.62 (m, 4H), 6.50-6.35 (m, 3H), 6.18-6.10 (m, 1H), 4.90 (s, 2H), 4.28 (d, J=2.7 Hz, 1H), 3.98-3.85 (m, 4H), 3.82 (s, 3H), 3.38 (dd, J=12.5, 12.5 Hz, 1H), 2.69 (d, J=11.6 Hz, 1H).

### Step 2

B10 (1.6 g, 2.7 mmol) was dissolved in THF/MeOH (1:2 v/v, 30 mL), and an aqueous NaOH solution (5 M, 4 mL) was added. The mixture was stirred at room temperature for 30 min. The solvent was removed by concentration under reduced pressure, and the mixture was adjusted to neutral with 2N HCl. Then, the mixture was extracted with ethyl acetate (50 mL×3), dried over anhydrous sodium sulfate, filtered, and concentrated under reduced pressure to remove the solvent to obtain a crude product. The intermediate obtained was dissolved in dichloromethane (100 mL), and triethylamine (0.54 mL, 4.1 mmol) was added. The mixture was cooled in an ice-water bath, and trifluoromethanesulfonic anhydride (0.6 mL, 3.2 mmol) was added dropwise. After stirring for 10 minutes, the mixture was quenched with water, and then separated. The organic phase was dried over anhydrous sodium sulfate, filtered, and concentrated under reduced pressure to remove the solvent to obtain a crude product. B11 (1.7 g, 98%) was obtained by column chromatography (petroleum ether/ethyl acetate 5:1). ¹H NMR (400 MHz, CDCl₃) δ 7.34 (d, J=8.4 Hz, 2H), 7.17 (d, J=2.5 Hz, 1H), 7.05 (d, J=8.6 Hz, 1H), 6.97-6.84 (m, 3H), 6.76-6.65 (m, 3H), 6.52-6.39 (m, 3H), 6.20-6.11 (m, 1H), 4.91 (s, 2H), 4.37-4.30 (m, 1H), 3.97-3.78 (m, 7H), 3.41 (dd, J=12.5, 12.5 Hz, 1H), 2.74 (d, J=11.7 Hz, 1H).

### Step 3

Under nitrogen atmosphere, B11 (0.70 g, 1.1 mmol), biboronic acid pinacol ester (0.42 g, 1.7 mmol), potassium acetate (0.33 g, 3.3 mmol), Pd(dppf)Cl₂ (90 mg, 0.11 mmol) and 1,4-dioxane (30 mL) were added to a reaction bottle. The reaction mixture was placed in an oil bath at 100° C. for 16 hours. After the reaction solution was cooled, it was concentrated under reduced pressure to remove the solvent to obtain a crude product, which was separated by column chromatography (petroleum ether/ethyl acetate 5:1) to obtain B12 (0.58 g, 86%). ¹H NMR (400 MHz, CDCl₃) δ7.74 (s, 1H), 7.41 (d, J=7.5 Hz, 1H), 7.33 (d, J=8.5 Hz, 2H), 7.01 (d, J=7.6 Hz, 1H), 6.91 (d, J=8.5 Hz, 2H), 6.73-6.63 (m, 3H), 6.51-6.37 (m, 3H), 6.16 (t, J=7.5 Hz, 1H), 4.90 (s, 2H), 4.34-4.27 (m, 1H), 3.97 (d, J=12.9 Hz, 1H), 3.85 (s, 3H), 3.82 (s, 3H), 3.41 (dd, J=12.5, 12.5 Hz, 1H), 2.70 (d, J=11.7 Hz, 1H), 1.37 (s, 12H).

### Step 4

The obtained B12 (0.50 g, 0.82 mmol) was dissolved in 1,4-dioxane (10 mL), and HCl (4M 1,4-dioxane solution, 4 mL) was added, and the mixture was reacted at room temperature for 30 min. The solvent was removed by concentration under reduced pressure to obtain a crude product, which was separated by column chromatography (petroleum ether/ethyl acetate 3:1) to obtain B13 (0.41 g, two-step yield 42%). ¹H NMR (400 MHz, CDCl₃) δ 7.71 (s, 1H), 7.38 (d, J=7.6 Hz, 1H), 6.98 (d, J=7.6 Hz, 1H), 6.64 (dd, J=10.9, 2.5 Hz, 1H), 6.54 (d, J=8.3 Hz, 2H), 6.44-6.37 (m, 3H), 6.15 (dd, J=8.5, 6.7 Hz, 1H), 4.82 (s, 1H), 4.28 (d, J=3.3 Hz, 1H), 3.99-3.89 (m, 1H), 3.84 (s, 3H), 3.38 (dd, J=12.5, 12.5 Hz, 1H), 2.68 (d, J=11.9 Hz, 1H), 1.35 (s, 12H).

### Step 5

B13 (300 mg, 0.61 mmol) was dissolved in acetonitrile (15 mL), 1,2-dibromoethane (2 mL) and potassium carbonate (420 mg, 3.0 mmol) were added, and the mixture was refluxed in an oil bath for 12 h, and the insoluble matter was removed by filtration, and the solvent was removed by decompression concentration to obtain a crude product. The obtained crude product was dissolved in acetonitrile (15 mL), 3-fluoromethyl-azetidine hydrochloride (140 mg, 1.1 mmol) and potassium carbonate (380 mg, 2.75 mmol) were added, and the insoluble matter was removed by filtration, and the solvent was removed by concentration under reduced pressure to obtain a crude product. The obtained crude product was dissolved in acetonitrile (9 mL), water (9 mL) and concentrated hydrochloric acid (0.5 mL), and stirred at room temperature for 12 h. The reaction solution was extracted with dichloromethane, dried over anhydrous sodium sulfate, filtered and concentrated, and separated by column chromatography (dichloromethane/methanol 30:1-10:1) to obtain Example 10 (51 mg, total yield of three steps was 16%). ¹H NMR (500 MHz, CDCl₃) δ 7.83 (s, 1H), 7.52 (s, 1H), 7.02-6.87 (m, 1H), 6.69-6.38 (m, 6H), 6.16 (t, J=7.9 Hz, 1H), 4.58-4.38 (m, 2H), 4.29 (s, 1H), 4.16-3.92 (m, 3H), 3.91-3.64 (m, 5H), 3.54-3.29 (m, 3H), 3.11-2.87 (m, 3H), 2.71 (d, J=10.8 Hz, 1H).

### Example 11

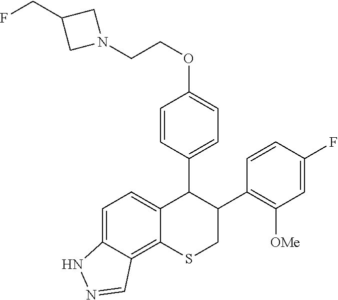 [SMILES: COc1cc(F)ccc1C1CSc2c(ccc3[nH]ncc23)C1c1ccc(OCCN2CC(CF)C2)cc1]

### Cis-3-(4-fluoro-2-methoxyphenyl)-4-(4-(2-(3-(fluoromethyl)azetidin-1-yl)ethoxy)phenyl)-2,3,4,7-tetrahydrothiopyrano[2,3-e]indazole

### Synthetic Route:

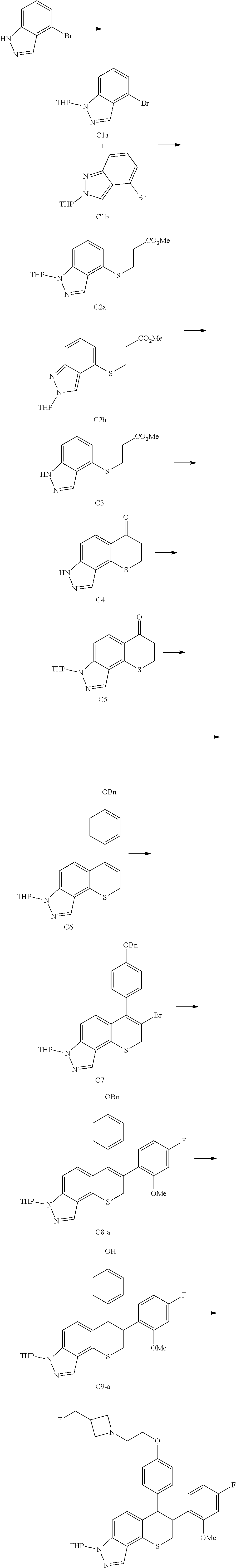

### Step 1

4-Bromoindazole (5.0 g, 25.4 mmol) and 2,3-dihydropyran (6.9 mL, 71 mmol) were dissolved in dichloromethane (100 mL), p-toluenesulfonic acid monohydrate (0.48 g, 2.5 mmol) was added, and the mixture was stirred at room temperature for 1 h. The reaction solution was washed with saturated sodium bicarbonate solution (100 mL), separated, and the aqueous phase was extracted with dichloromethane (100 mL). The organic phases were combined and dried over anhydrous sodium sulfate, filtered, and concentrated under reduced pressure to remove the solvent to obtain a white solid as a mixture of C1a and C1b (7.1 g, 100%). LCMS: 281.2 [M+H].

### Step 2

Under nitrogen atmosphere, a mixture of C1a and C1b (7.0 g, 25 mmol), methyl 3-mercaptopropionate (3.6 g, 30 mmol), Pd₂(dba)₃ (2.3 g, 2.5 mmol), Xanphos (2.9 g, 5.0 mmol) and DIPEA (8.0 g, 62.5 mmol) were added in a reaction flask, and 1,4-dioxane (100 mL) was added. After reacting under reflux for 72 hours, the crude product was filtered through diatomaceous earth and concentrated under reduced pressure to remove the solvent, and the crude product containing C2a and C2b was collected by silica gel filtration (eluent: petroleum ether/ethyl acetate 10:1-3:1) and used directly in the next step. LCMS: 321.3 [M+H].

### Step 3

The mixture of C2a and C2b obtained in the previous step was dissolved in 1,4-dioxane (20 mL), and water (9 mL) and concentrated hydrochloric acid (5 mL) were added, and the mixture was reacted in an oil bath at 80° C. for 2 hours. The solvent was removed by concentration under reduced pressure to obtain a crude product, and toluene (30 mL) was added and the mixture was concentrated, and the reaction was repeated twice to obtain an oily product C3, which was used directly in the next step.

### Step 4

Eaton's reagent (CAS #: 39394-84-8; 40 mL) was added to the crude product C3 obtained in the previous step, and the mixture was reacted in an oil bath at 80° C. for 0.5 hours. The reaction solution was cooled to room temperature and poured into ice water, extracted with ethyl acetate (100 mL×2), and the organic phase was washed with saturated sodium bicarbonate solution (100 mL) and saturated sodium chloride solution (100 mL) in sequence, dried over anhydrous sodium sulfate, filtered, and concentrated under reduced pressure to remove the solvent to obtain a crude product, and petroleum ether/ethyl acetate (2:1 v/v, 20 mL) was added and stirred, and the mixture was filtered to obtain C4 (3.0 g, three-step yield 60%) as a yellow solid. ¹H NMR (400 MHz, CDCl₃) δ8.20 (s, 1H), 8.14 (d, J=8.9 Hz, 1H), 7.24 (d, J=8.9 Hz, 1H), 3.43-3.35 (m, 2H), 3.08-3.00 (m, 2H).

### Step 5

C4 (3.0 g, 14.7 mmol) and 2,3-dihydropyran (2.8 mL, 29 mmol) were dissolved in tetrahydrofuran (50 mL), p-toluenesulfonic acid monohydrate (0.31 g, 1.5 mmol) was added, and the mixture was refluxed in an oil bath for 12 h. The solvent was removed by concentration under reduced pressure and the residue was then separated by column chromatography (petroleum ether/ethyl acetate 5:1-3:1) to obtain C5 (3.4 g, 81%). ¹H NMR (400 MHz, CDCl₃) δ 8.19-8.08 (m, 2H), 7.35 (d, J=9.0 Hz, 1H), 5.70 (dd, J=9.3, 2.6 Hz, 1H), 4.08-3.97 (m, 1H), 3.81-3.68 (m, 1H), 3.43-3.32 (m, 2H), 3.09-2.96 (m, 2H), 2.59-2.44 (m, 1H), 2.22-2.01 (m, 2H), 1.88-1.62 (m, 4H).

### Step 6

C5 (1.8 g, 6.7 mmol) and 2,3-dihydropyran (1.7 g, 9.1 mmol) were dissolved in ethanol (20 mL) and refluxed in an oil bath for 6 h. After cooling to room temperature, the mixture was concentrated to remove the solvent. The crude product was dissolved in 1,4-dioxane (100 mL), and 4-benzyloxybromobenzene (2.2 g, 7.9 mmol), Pd₂(dba)₃ (313 mg, 0.33 mmol), Xanphos (330 mg, 0.66 mmol) and lithium tert-butoxide (1.2 g, 14.5 mmol) were added. After reacting for 2 hours under reflux conditions, the mixture was cooled to room temperature, diluted with ethyl acetate (100 mL), then filtered with diatomaceous earth, and concentrated under reduced pressure to remove the solvent to obtain a crude product, which was separated by column chromatography (petroleum ether/ethyl acetate 4:1) to obtain C7 (1.6 g, 55%). ¹H NMR (400 MHz, CDCl₃) δ 8.14 (s, 1H), 7.53-7.35 (m, 5H), 7.33-7.20 (m, 3H), 7.16 (d, J=8.7 Hz, 1H), 7.00 (d, J=8.5 Hz, 2H), 5.94 (t, J=5.8 Hz, 1H), 5.76-5.66 (m, 1H), 5.12 (s, 2H), 4.12-4.00 (m, 1H), 3.81-3.70 (m, 1H), 3.51 (d, J=5.8 Hz, 2H), 2.67-2.51 (m, 1H), 2.24-2.05 (m, 2H), 1.85-1.64 (m, 3H).

### Step 7

B4 (500 mg, 1.1 mmol) was dissolved in dichloromethane (50 mL), cooled in an ice-water bath, and pyridinium tribromide (450 mg, 1.2 mmol) was added. After stirring in an ice-water bath for 1 hour, the mixture was quenched with a saturated sodium bicarbonate solution (100 mL), separated, and the aqueous phase was extracted with dichloromethane (50 mL). The organic phases were combined, washed with a saturated sodium chloride solution (50 mL), dried over anhydrous sodium sulfate, filtered, and concentrated under reduced pressure to remove the solvent to obtain a crude product, which was separated by column chromatography (petroleum ether/ethyl acetate 5:1) to obtain C8 (460 mg, 78%). ¹H NMR (400 MHz, CDCl₃) δ 8.09 (s, 1H), 7.55-7.36 (m, 5H), 7.21-7.13 (m, 3H), 7.06 (d, J=8.7 Hz, 2H), 6.81 (d, J=8.9 Hz, 1H), 5.66 (dd, J=9.3, 2.7 Hz, 1H), 5.13 (s, 2H), 4.07-3.97 (m, 3H), 3.77-3.68 (m, 1H), 2.59-2.46 (m, 1H), 2.19-2.03 (m, 2H), 1.82-1.66 (m, 3H).

### Step 8

B5 (0.90 g, 1.87 mmol), 4-fluoro-2-methoxyphenylboronic acid (0.30 g, 1.6 mmol), Pd(PPh₃)₄ (202 mg, 0.17 mmol) and cesium carbonate (1.2 g, 3.7 mmol) were added to a reaction flask, and a mixture of 1,4-dioxane-water (4:1 v/v, 30 mL) was added, and the mixture was reacted in an oil bath at 80° C. for 4 hours under nitrogen protection. After the reaction solution was cooled, it was diluted with water (50 mL), extracted with ethyl acetate (50 mL×2), the organic phases were combined, washed with saturated sodium chloride solution (40 mL), dried over anhydrous sodium sulfate, filtered, and concentrated under reduced pressure to remove the solvent, and separated by column chromatography (petroleum ether/ethyl acetate 5:1) to obtain C9-a (0.89 g, 92%). ¹H NMR (500 MHz, CDCl₃) δ 8.10 (d, J=0.9 Hz, 1H), 7.44-7.31 (m, 5H), 7.18 (dd, J=8.8, 1.0 Hz, 1H), 6.96 (d, J=8.8 Hz, 1H), 6.88 (d, J=8.2 Hz, 2H), 6.82-6.73 (m, 3H), 6.55 (dd, J=10.9, 2.4 Hz, 1H), 6.35 (td, J=8.4, 2.4 Hz, 1H), 5.66 (dd, J=9.3, 2.7 Hz, 1H), 5.00 (s, 2H), 4.09-3.89 (m, 2H), 3.80 (s, 3H), 3.76-3.68 (m, 1H), 3.37 (s, 1H), 2.62-2.44 (m, 1H), 2.22-2.02 (m, 2H), 1.82-1.62 (m, 3H).

### Step 9

C9 (0.8 g, 1.4 mmol) was dissolved in ethanol (20 mL), and 20% Pd(OH)₂/C (0.8 g) was added, and the mixture was reacted under a hydrogen atmosphere (1 atm) at 50° C. for 24 h. The crude product was filtered through diatomaceous earth and concentrated under reduced pressure to remove the solvent. The crude product was separated by column chromatography (petroleum ether/ethyl acetate 1:1) to obtain a white solid C10-a (0.32 g, 48%). ¹H NMR (500 MHz, DMSO-d6) δ 9.14 (s, 1H), 8.02 (s, 1H), 7.29 (dd, J=8.6, 2.3 Hz, 1H), 6.97-6.90 (m, 2H), 6.51 (td, J=8.5, 2.5 Hz, 1H), 6.47-6.43 (m, 2H), 6.29-6.18 (m, 3H), 5.77 (dd, J=9.7, 2.6 Hz, 1H), 4.32-4.28 (m, 1H), 3.89-3.82 (m, 4H), 3.81-3.76 (m, 1H), 3.73-3.65 (m, 1H), 3.43-3.34 (m, 1H), 2.90 (d, J=12.0 Hz, 1H), 2.43-2.30 (m, 1H), 2.05-1.98 (m, 1H), 1.96-1.90 (m, 1H), 1.78-1.66 (m, 1H), 1.60-1.50 (m, J=3.8, 3.2 Hz, 2H).

### Step 10

C10 (200 mg, 0.408 mmol) was dissolved in acetonitrile (15 mL), and 1,2-dibromoethane (2 mL) and potassium carbonate (280 mg, 2.1 mmol) were added. After the mixture was refluxed in an oil bath for 12 h, the insoluble matter was removed by filtration, and the solvent was removed by concentration under reduced pressure. The crude product was used directly in the next step.

The crude product obtained in the previous step was dissolved in acetonitrile (15 mL), 3-fluoromethyl-azetidine hydrochloride (100 mg, 0.8 mmol) and potassium carbonate (380 mg, 2.75 mmol) were added. After the mixture was refluxed in an oil bath for 4 h, the insoluble matter was filtered off, and the solvent was removed by concentration under reduced pressure. The crude product was dissolved in isopropanol (6 mL), HCl (4M isopropanol solution, 2 mL) was added, and the reaction was carried out at 60° C. for 30 min, and then the solvent was removed by concentration under reduced pressure, and the product was dissolved in dichloromethane (50 mL), neutralized with saturated sodium bicarbonate solution (20 mL), and separated. The aqueous phase was extracted with dichloromethane (20 mL×2), and the organic phases were combined, and then dried over anhydrous sodium sulfate, filtered, and concentrated under reduced pressure to remove the solvent to obtain a crude product, which was separated by column chromatography (dichloromethane/methanol 30:1-10:1) to obtain a white solid Example 11 (120 mg, the total yield of three steps was 57%). ¹H NMR (500 MHz, CDCl₃) δ 10.80 (s, 1H), 8.12 (s, 1H), 7.04 (d, J=9.2 Hz, 1H), 6.91 (d, J=8.6 Hz, 1H), 6.65 (dd, J=10.9, 2.5 Hz, 1H), 6.56 (d, J=8.8 Hz, 2H), 6.47-6.36 (m, 3H), 6.12 (dd, J=8.4, 6.7 Hz, 1H), 4.48 (dd, J=47.4, 5.6 Hz, 2H), 4.36 (d, J=3.6 Hz, 1H), 4.04 (dt, J=12.9, 2.8 Hz, 1H), 3.90-3.81 (m, 5H), 3.52-3.45 (m, 3H), 3.14 (t, J=7.0 Hz, 2H), 2.91-2.76 (m, 4H).

### Example 12

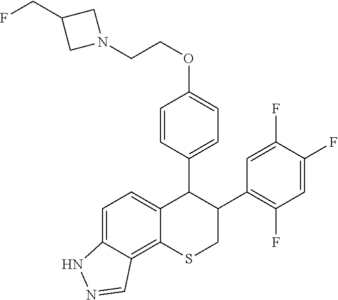 [SMILES: FCC1CN(CCOc2ccc(C3c4ccc5[nH]ncc5c4SCC3c3cc(F)c(F)cc3F)cc2)C1]

### Cis-4-(4-(2-(3-(fluoromethyl)azetidin-1-yl)ethoxy)phenyl)-3-(2,4,5-trifluorophenyl)-2,3,4,7-tetrahydrothiopyrano[2,3-e]indazole

### Synthetic Route:

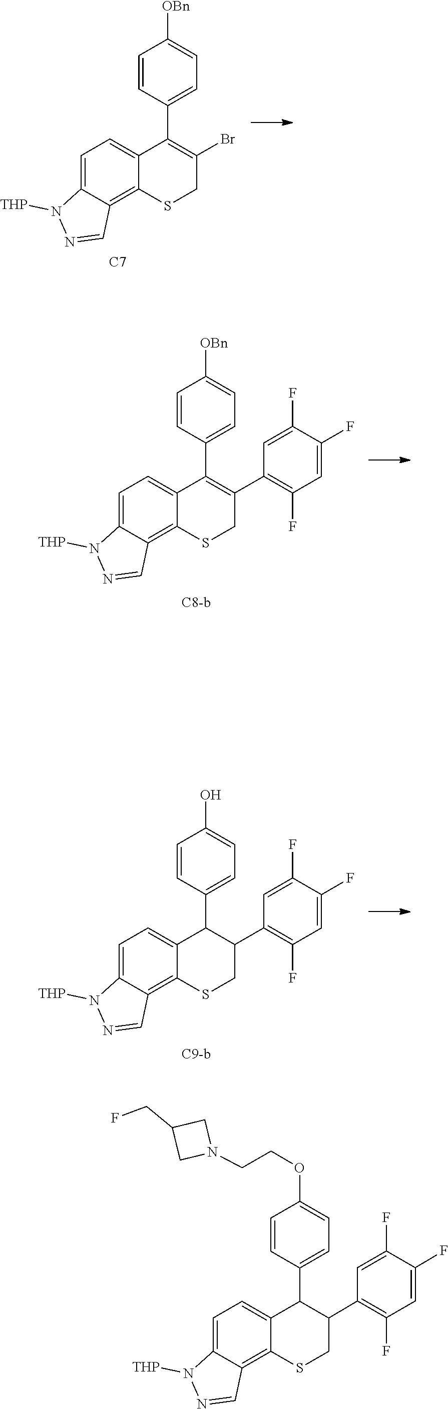

### Step 1

C8 (0.40 g, 0.75 mmol), 2,4,5-trifluorophenylboronic acid (0.16 g, 0.90 mmol), Pd(PPh₃)₄ (90 mg, 0.08 mmol) and cesium carbonate (0.50 g, 1.56 mmol) were added to a reaction flask, and a mixture of 1,4-dioxane-water (4:1 v/v, 15 mL) was added, and the mixture was reacted in an oil bath at 80° C. for 4 hours under nitrogen protection. After the reaction solution was cooled, it was diluted with water (20 mL) and extracted with ethyl acetate (50 mL×2). The organic phases were combined, washed with saturated sodium chloride solution (40 mL), dried over anhydrous sodium sulfate, filtered, concentrated under reduced pressure to remove the solvent, and separated by column chromatography (petroleum ether/ethyl acetate 5:1) to obtain C9-b (0.35 g, 80%).

### Step 2

C9 (0.35 g, 0.30 mmol) was dissolved in ethanol (15 mL), and 20% Pd(OH)₂/C (0.4 g) was added, and the mixture was reacted under a hydrogen atmosphere (1 atm) at 50° C. for 8 h. The mixture was filtered through diatomaceous earth and concentrated under reduced pressure to remove the solvent. The crude product was separated by column chromatography (petroleum ether/ethyl acetate 2:1) to obtain a white solid C10-b (145 mg, 49%). ¹H NMR (400 MHz, CDCl₃) δ8.08 (s, 1H), 7.23 (t, J=8.3 Hz, 1H), 7.06-6.95 (m, 2H), 6.69-6.46 (m, 4H), 6.23-6.11 (m, 1H), 5.70 (s, 1H), 4.36 (d, J=3.8 Hz, 1H), 4.02 (dd, J=26.5, 12.4 Hz, 1H), 3.82-3.71 (m, 2H), 3.47 (t, J=12.4 Hz, 1H), 2.85 (d, J=12.0 Hz, 1H), 2.66-2.52 (m, 1H), 2.24-2.02 (m, 2H), 1.72 (d, J=40.4 Hz, 3H).

### Step 3

C10 (140 mg, 0.282 mmol) was dissolved in acetonitrile (15 mL), 1,2-dibromoethane (2 mL) and potassium carbonate (195 mg, 1.41 mmol) were added. After the mixture was refluxed in an oil bath for 12 h, the insoluble matter was removed by filtration, and the solvent was removed by concentration under reduced pressure. The crude product was directly used in the next step.

The crude product obtained in the previous step was dissolved in acetonitrile (15 mL), 3-fluoromethyl-azetidine hydrochloride (71 mg, 0.56 mmol) and potassium carbonate (195 mg, 1.41 mmol) were added. After the mixture was refluxed in an oil bath for 4 h, the insoluble matter was removed by filtration, and the solvent was removed by concentration under reduced pressure. The crude product was dissolved in isopropanol (6 mL), HCl (4M isopropanol solution, 2 mL) was added, and the reaction was carried out at 60° C. for 1 h, and then the solvent was removed by concentration under reduced pressure, and the product was dissolved in dichloromethane (50 mL), neutralized with saturated sodium bicarbonate solution (20 mL), and separated. The aqueous phase was extracted with dichloromethane (20 mL×2), and the organic phases were combined, and then dried over anhydrous sodium sulfate, filtered, and concentrated under reduced pressure to remove the solvent to obtain a crude product, which was separated by column chromatography (dichloromethane/methanol 30:1-10:1) to obtain Example 12 as a white solid (65 mg, the total yield of three steps was 44%). ¹H NMR (500 MHz, CDCl₃) δ 11.14 (s, 1H), 8.14 (s, 1H), 7.07 (d, J=8.5 Hz, 1H), 6.99 (q, J=9.5 Hz, 1H), 6.91 (d, J=8.5 Hz, 1H), 6.64 (d, J=8.3 Hz, 2H), 6.53 (d, J=8.3 Hz, 2H), 6.14 (q, J=8.4 Hz, 1H), 4.51 (dd, J=47.4, 5.5 Hz, 2H), 4.36 (d, J=3.1 Hz, 1H), 4.04-3.87 (m, 3H), 3.58-3.42 (m, 3H), 3.19 (t, J=7.0 Hz, 2H), 2.88 (dt, J=22.1, 6.3 Hz, 4H).

### Example 13

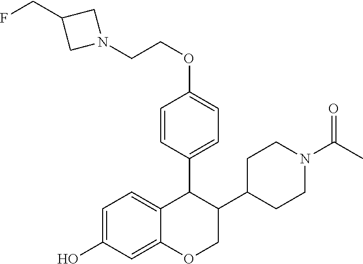 [SMILES: CC(=O)N1CCC(C2COc3cc(O)ccc3C2c2ccc(OCCN3CC(CF)C3)cc2)CC1]

### Cis-1-(4-(4-(2-(3-(fluoromethyl)azetidin-1-yl)ethoxy)phenyl)-7-hydroxychroman-3-yl)piperidin-1-yl ethanone

### Synthetic Route:

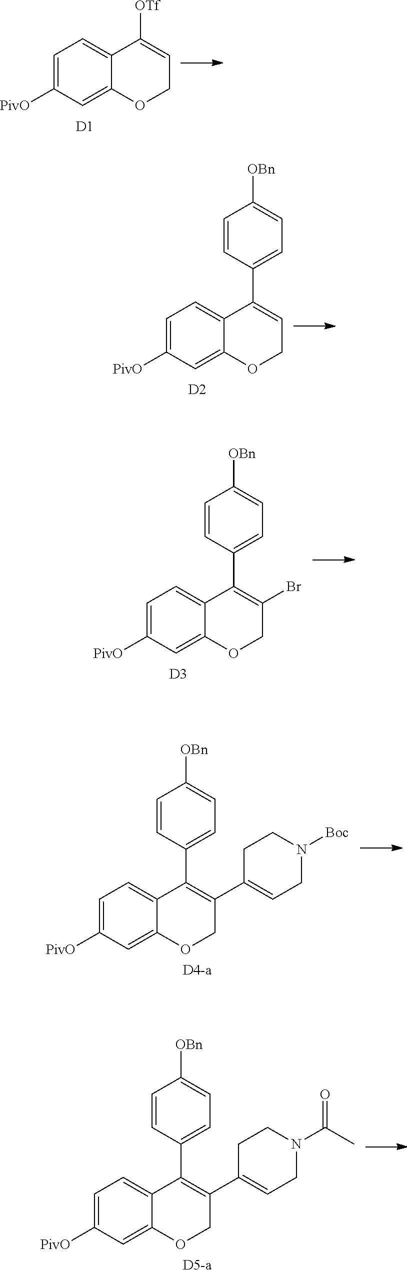

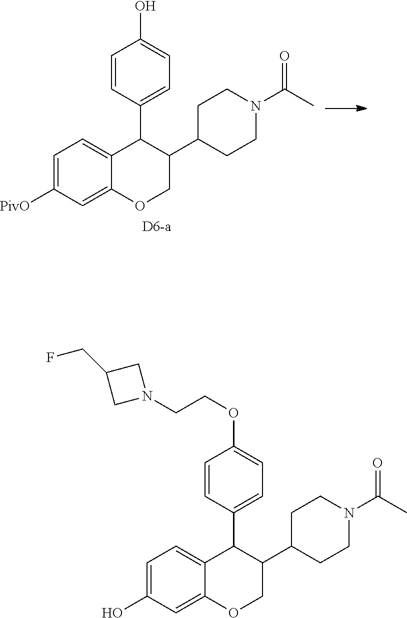

Step 1: D1 (3.8 g, 10 mmol) (reference patent: WO2018091153), 4-benzyloxyphenylboronic acid (2.7 g, 11.8 mmol), Pd(dppf)Cl₂ (0.81 g, 1.0 mmol) and cesium carbonate (6.5 g, 20 mmol) were added to a reaction flask. 1,4-dioxane-water mixture (4:1 v/v, 50 mL) was added and reacted in a 50° C. oil bath under nitrogen protection for 1 hour. The mixture was diluted with water (50 mL) and extracted with ethyl acetate (50 mL×2). The combined organic phases was washed with saturated sodium chloride solution (100 mL), dried over anhydrous sodium sulfate, filtered, and concentrated under reduced pressure to remove the solvent. The residue was separated by column chromatography (petroleum ether/ethyl acetate 20:1) to obtain a white solid D2 (3.7 g, 90%). ¹H NMR (400 MHz, CDCl₃) δ 7.51-7.35 (m, 5H), 7.31-7.27 (m, 2H), 7.06-7.00 (m, 3H), 6.65 (d, J=2.3 Hz, 1H), 6.58 (dd, J=8.4, 2.3 Hz, 1H), 5.74 (t, J=4.0 Hz, 1H), 5.13 (s, 2H), 4.86 (d, J=4.0 Hz, 2H), 1.37 (s, 9H).
Step 2: D2 (5.0 g, 12.0 mmol) was dissolved in dichloromethane (200 mL), cooled in an ice-water bath, and pyridinium tribromide (4.5 g, 12.0 mmol, 85% content) was added. After stirring in an ice-water bath for 1 hour, the mixture was quenched with saturated sodium bicarbonate solution (100 mL) and separated. The aqueous phase was extracted with dichloromethane (100 mL), The organic phases were combined, washed with saturated sodium chloride solution (100 mL), dried over anhydrous sodium sulfate, filtered, and concentrated under reduced pressure to remove the solvent to obtain a crude product, which was separated by column chromatography (petroleum ether/ethyl acetate 10:1) to obtain a white solid D3 (5.4 g, 85%). ¹H NMR (400 MHz, CDCl₃) δ 7.53-7.36 (m, 5H), 7.26-7.20 (m, 2H), 7.12-7.06 (m, 2H), 6.73 (d, J=8.4 Hz, 1H), 6.64 (d, J=2.3 Hz, 1H), 6.54 (dd, J=8.4, 2.3 Hz, 1H), 5.14 (s, 2H), 5.05 (s, 2H), 1.37 (s, 9H).
Step 3: D3 (1.0 g, 2.03 mmol), N-Boc-1,2,5,6-tetrahydropyridine-4-boronic acid pinacol ester (0.75 g, 2.44 mmol), Pd(PPh₃)₄ (230 mg, 0.2 mmol) and cesium carbonate (1.6 g, 4.0 mmol) were added to a reaction flask, and a mixture of 1,4-dioxane-water (4:1 v/v, 20 mL) was added, and the mixture was reacted in an oil bath at 100° C. for 4 hours under nitrogen protection. After the reaction solution was cooled, it was diluted with water (50 mL), extracted with ethyl acetate (50 mL×2), the organic phases were combined, washed with saturated sodium chloride solution (40 mL), dried over anhydrous sodium sulfate, filtered, and concentrated under reduced pressure to remove the solvent, and separated by column chromatography (petroleum ether/ethyl acetate 5:1) to obtain a white solid D4-a (1.1 g, 91%). ¹H NMR (400 MHz, CDCl₃) δ 7.44 (ddd, J=25.8, 19.8, 7.2 Hz, 5H), 7.13 (d, J=8.5 Hz, 2H), 7.00 (d, J=8.6 Hz, 2H), 6.77 (d, J=8.4 Hz, 1H), 6.63 (d, J=2.3 Hz, 1H), 6.51 (dd, J=8.4, 2.3 Hz, 1H), 5.50 (s, 1H), 5.12 (s, 2H), 4.90 (s, 2H), 3.89 (s, 2H), 3.27 (s, 2H), 1.80 (s, 2H), 1.47 (s, 9H), 1.36 (s, 9H).
Step 4: D4-a (0.50 g, 0.84 mmol) was dissolved in 1,4-dioxane (5 mL), HCl (4M 1,4-dioxane solution, 2 mL) was added, and the mixture was stirred at room temperature for 30 min. The solvent was removed by concentration, and the crude product was dissolved in dichloromethane (100 mL), washed with saturated sodium bicarbonate solution, separated, and the organic phase was dried over anhydrous sodium sulfate, filtered, and concentrated under reduced pressure to remove the solvent to obtain the intermediate. The intermediate was dissolved in dichloromethane (10 mL), triethylamine (170 mg, 1.7 mmol) and acetyl chloride (98 mg, 1.26 mmol) were added, and the mixture was stirred at room temperature for 30 min. The concentrated crude product was separated by column chromatography (petroleum ether/ethyl acetate 1:1) to obtain D5-a (0.38 g, two-step yield was 84%). ¹H NMR (400 MHz, CDCl₃) δ7.52-7.34 (m, 5H), 7.15-7.08 (m, 2H), 7.04-6.97 (m, 2H), 6.76 (d, J=8.4 Hz, 1H), 6.63 (d, J=2.2 Hz, 1H), 6.52 (dd, J=8.4, 2.3 Hz, 1H), 5.63-5.57 (m, 0.6H) & 5.49-5.44 (m, 0.4H) (═CH—), 5.12 (s, 1.2H) & 5.12 (s, 0.8H) (—C 9H2 —O), 4.90 (s, 1.2H) & 4.89 (s, 0.8H) (—CH2 —O) (—CH2 —O), 4.07 (q, J=2.7 Hz, 1.2H) & 3.93 (q, J=2.7 Hz, 0.8H) (—CH2 —N), 3.47 (t, J=5.6 Hz, 0.8H) & 3.27 (t, J=5.5 Hz, 1.2H) (—CH2 —N), 2.06 (s, 1.2H) & 2.04 (s, 1.8H) (CH3 CO—), 1.85-1.78 (m, 2H), 1.35 (s, 9H).
Step 5: D5-a (0.38 g) was dissolved in ethanol (15 mL), 20% Pd(OH)₂/C (0.5 g) was added, and the mixture was reacted under a hydrogen atmosphere (1 atm) at 60° C. for 8 h. The mixture was filtered through diatomaceous earth and concentrated under reduced pressure to remove the solvent, and then separated by column chromatography (petroleum ether/ethyl acetate 1:2) to obtain D6-a (0.21 g, 66%). ¹H NMR (400 MHz, CDCl₃) δ 6.97-6.88 (m, 3H), 6.81-6.75 (m, 2H), 6.61 (d, J=2.3 Hz, 1H), 6.52 (dd, J=8.3, 1.8 Hz, 1H), 4.69-4.46 (m, 1H), 4.37-3.99 (m, 4H), 3.89-3.64 (m, 1H), 3.03-2.78 (m, 1H), 2.55-2.28 (m, 1H), 2.17-2.01 (m, 6H), 1.78-1.42 (m, 1H), 1.35 (s, 9H), 1.17-1.03 (m, 1H).
Step 6: D6-a (170 mg, 0.38 mmol) was dissolved in acetonitrile (15 mL), 1,2-dibromoethane (2 mL) and potassium carbonate (260 mg, 1.9 mmol) were added and refluxed in an oil bath for 12 h. The mixture was filtered to remove the insoluble matter and then concentrated under reduced pressure to remove the solvent to. The obtained crude product was then dissolved in acetonitrile (15 mL), 3-fluoromethyl-azetidine hydrochloride (95 mg, 0.76 mmol) and potassium carbonate (260 mg, 1.9 mmol) were added. After the mixture was refluxed in an oil bath for 1 h, the insoluble matter was filtered out and the solvent was removed. The crude product was dissolved in methanol (5 mL), sodium methoxide (0.5 mL) was added, and the mixture was stirred at room temperature for 10 min, then concentrated under reduced pressure to remove the solvent, neutralized with 1N HCl to pH 7-8, and extracted with dichloromethane (15 mL×3). The organic phases were combined, and then dried over anhydrous sodium sulfate, filtered, and concentrated under reduced pressure to remove the solvent to obtain a crude product, which was separated by column chromatography (dichloromethane/methanol 20:1-10:1) to obtain Example 13 (108 mg, total yield of three steps was 60%). ¹H NMR (500 MHz, CDCl₃) δ 6.95 (dd, J=8.7, 3.2 Hz, 2H), 6.72-6.65 (m, 3H), 6.33 (d, J=2.5 Hz, 1H), 6.29 (dd, J=8.2, 2.5 Hz, 1H), 4.68-4.41 (m, 3H), 4.21-3.90 (m, 6H), 3.86-3.58 (m, 4H), 3.36-3.25 (m, 2H), 3.01-2.76 (m, 4H), 2.50-2.26 (m, 1H), 2.16-1.91 (m, 6H), 1.13-1.00 (m, 1H).

### Example 14

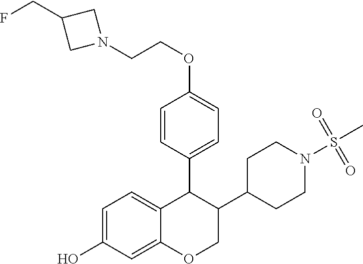 [SMILES: CS(=O)(=O)N1CCC(C2COc3cc(O)ccc3C2c2ccc(OCCN3CC(CF)C3)cc2)CC1]

### Cis-4-(4-(2-(3-(fluoromethyl)azetidin-1-yl)ethoxy)phenyl)-3-(1-(methylsulfonyl)piperidin-4-yl)-7-hydroxychroman

### Synthetic Route:

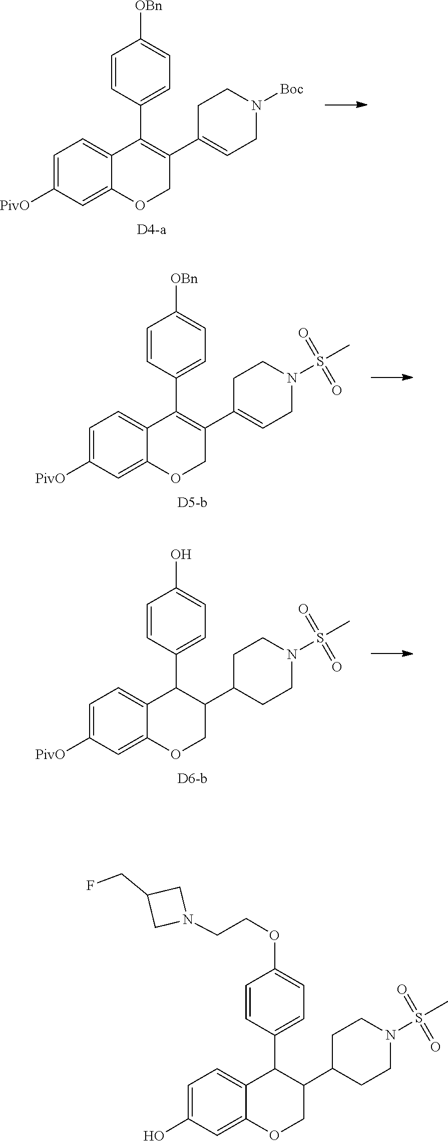

Step 1: D4-a (0.60 g, 1.01 mmol) was dissolved in 1,4-dioxane (5 mL), add HCl (4M 1,4-dioxane solution, 2 mL), and stir at room temperature for 30 min. Concentrate to remove the solvent, dissolve the crude product in dichloromethane (100 mL), wash with saturated sodium bicarbonate solution, separate the liquids, dry the organic phase with anhydrous sodium sulfate, filter, and concentrate under reduced pressure to remove the solvent to obtain the intermediate. The intermediate is dissolved in dichloromethane (10 mL), triethylamine (202 mg, 2.0 mmol) and methanesulfonic anhydride (186 mg, 1.5 mmol) are added, and stir at room temperature for 10 min. The concentrated crude product is separated by column chromatography (petroleum ether/ethyl acetate 1:1) to obtain D5-b (0.57 g, two-step yield is 99%). ¹H NMR (400 MHz, CDCl₃) δ 7.52-7.36 (m, 5H), 7.11 (d, J=8.7 Hz, 2H), 7.00 (d, J=8.7 Hz, 2H), 6.74 (d, J=8.4 Hz, 1H), 6.63 (d, J=2.3 Hz, 1H), 6.52 (dd, J=8.4, 2.3 Hz, 1H), 5.58-5.52 (m, 1H), 5.12 (s, 2H), 4.88 (s, 2H), 3.79 (q, J=2.8 Hz, 2H), 3.18 (t, J=5.6 Hz, 2H), 2.68 (s, 3H), 1.97-1.90 (m, 2H), 1.36 (s, 9H).
Step 2: D5-b (0.54 g) was dissolved in ethanol (15 mL), 20% Pd(OH)₂/C (0.5 g) was added and reacted for 8 h at 60° C. in a hydrogen atmosphere (1 atm). The mixture was filtered with diatomaceous earth and concentrated under reduced pressure to remove the solvent to obtain a crude product, which was separated by column chromatography (petroleum ether/ethyl acetate 1:1) to obtain D6-b (0.37 g, 80%). ¹H NMR (400 MHz, CDCl₃) δ 6.95 (d, J=8.5 Hz, 2H), 6.89 (d, J=8.4 Hz, 1H), 6.74 (d, J=8.5 Hz, 2H), 6.61 (d, J=2.3 Hz, 1H), 6.52 (dd, J=8.3, 2.3 Hz, 1H), 4.25 (dd, J=11.0, 2.9 Hz, 1H), 4.13 (d, J=4.7 Hz, 1H), 4.05 (dd, J=11.4 Hz, 1H), 3.80 (d, J=11.6, 11.6 Hz, 1H), 3.67 (d, J=11.5 Hz, 1H), 2.74 (s, 3H), 2.62-2.51 (m, 1H), 2.47-2.37 (m, 1H), 2.19-2.04 (m, 2H), 1.52-1.44 (m, 1H), 1.38-1.26 (m, 11H), 1.23-1.12 (m, 1H).
Step 3: D6-b (250 mg, 0.51 mmol) was dissolved in acetonitrile (15 mL), 1,2-dibromoethane (2 mL) and potassium carbonate (350 mg, 2.6 mmol) were added, refluxed in an oil bath for 12 h, filtered to remove the insoluble matter, and concentrated under reduced pressure to remove the solvent to obtain a crude product. The crude product was then dissolved in acetonitrile (15 mL), 3-fluoromethyl-azetidine hydrochloride (140 mg, 1.1 mmol) and potassium carbonate (350 mg, 2.6 mmol) were added. After the mixture was refluxed in an oil bath for 1 h, the insoluble matter was filtered off, and the solvent was removed by concentration under reduced pressure. The crude product was dissolved in methanol (5 mL), sodium methoxide (0.5 mL) was added, and the mixture was stirred at room temperature for 10 min, and then concentrated under reduced pressure to remove the solvent. The mixture was neutralized with 1N HCl to pH 7-8, and extracted with dichloromethane (15 mL×3). The organic phases were combined, and then dried over anhydrous sodium sulfate, filtered, and concentrated under reduced pressure to remove the solvent to obtain a crude product, which was separated by column chromatography (dichloromethane/methanol 20:1-10:1) to obtain Example 14 (140 mg, total yield of three steps was 54%). ¹H NMR (600 MHz, CDCl₃) δ 6.95 (d, J=8.7 Hz, 2H), 6.70-6.64 (m, 3H), 6.35 (d, J=2.4 Hz, 1H), 6.28 (dd, J=8.3, 2.5 Hz, 1H), 4.51 (dd, J=47.4, 5.2 Hz, 2H), 4.22-4.16 (m, 1H), 4.07 (d, J=4.8 Hz, 1H), 4.01-3.90 (m, 3H), 3.81 (d, J=12.5 Hz, 1H), 3.68 (d, J=11.6 Hz, 1H), 3.59 (t, J=7.7 Hz, 2H), 3.29-3.22 (m, 2H), 3.01-2.87 (m, 3H), 2.74 (s, 3H), 2.55 (td, J=12.0, 2.6 Hz, 1H), 2.42 (td, J=11.5, 3.4 Hz, 1H), 2.17 (d, J=13.9 Hz, 1H), 2.07 (ddt, J=12.4, 8.8, 4.5 Hz, 1H), 1.47 (qd, J=12.2, 4.2 Hz, 1H), 1.37-1.27 (m, 2H), 1.15-1.08 (m, 1H).

### Example 15

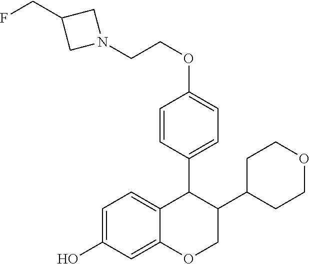 [SMILES: Oc1ccc2c(c1)OCC(C1CCOCC1)C2c1ccc(OCCN2CC(CF)C2)cc1]

### Cis-4-(4-(2-(3-(3-(fluoromethyl)azetidin-1-yl)ethoxy)phenyl)-3-(tetrahydro-2H-pyran-4-yl)-7-hydroxychroman

### Synthetic Route:

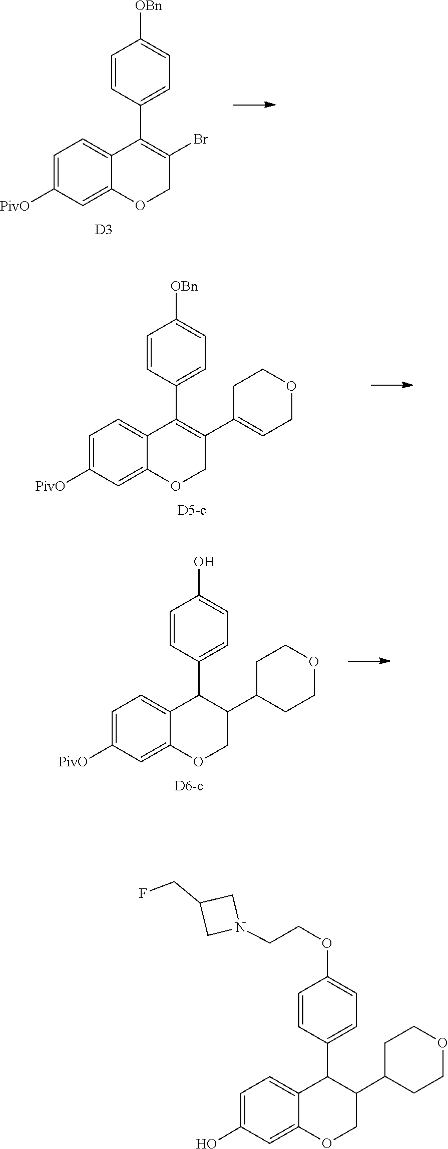

Step 1: D3 (1.0 g, 2.03 mmol), 3,6-dihydro-2H-pyran-4-boronic acid pinacol ester (0.52 g, 2.44 mmol), Pd(PPh₃)₄ (230 mg, 0.2 mmol) and cesium carbonate (1.6 g, 4.0 mmol) were added to a reaction flask, and a mixture of 1,4-dioxane-water (4:1 v/v, 20 mL) was added, and the mixture was reacted in an oil bath at 100° C. for 4 hours under nitrogen protection. After the reaction solution was cooled, it was diluted with water (50 mL), extracted with ethyl acetate (50 mL×2). The organic phases were combined, washed with saturated sodium chloride solution (40 mL), dried over anhydrous sodium sulfate, filtered, and concentrated under reduced pressure to remove the solvent, and separated by column chromatography (petroleum ether/ethyl acetate 5:1) to obtain a white solid D5-c (0.86 g, 85%). ¹H NMR (400 MHz, CDCl₃) δ 7.52-7.34 (m, 5H), 7.15 (d, J=8.7 Hz, 2H), 7.01 (d, J=8.7 Hz, 2H), 6.77 (d, J=8.4 Hz, 1H), 6.63 (d, J=2.1 Hz, 1H), 6.52 (dd, J=8.4, 2.2 Hz, 1H), 5.55 (s, 1H), 5.12 (s, 2H), 4.91 (s, 2H), 4.15 (d, J=2.6 Hz, 2H), 3.59 (t, J=5.3 Hz, 2H), 1.84-1.76 (m, 2H), 1.36 (s, 9H).
Step 2: D5-c (0.51 g) was dissolved in ethanol (15 mL), and 20% Pd(OH)₂/C (0.6 g) was added, and the mixture was reacted under a hydrogen atmosphere (1 atm) at 60° C. for 8 h. The crude product was filtered through diatomaceous earth and concentrated under reduced pressure to remove the solvent, and then separated by column chromatography (petroleum ether/ethyl acetate 3:1) to obtain D6-c (0.20 g, 47%). ¹H NMR (400 MHz, DMSO-d6) δ 9.31 (s, 1H), 6.90 (d, J=8.3 Hz, 3H), 6.68 (d, J=8.5 Hz, 2H), 6.57 (d, J=2.3 Hz, 1H), 6.50 (dd, J=8.3, 2.3 Hz, 1H), 4.28 (dd, J=10.9, 3.1 Hz, 1H), 4.15 (d, J=4.3 Hz, 1H), 3.97 (t, J=11.7 Hz, 1H), 3.88-3.81 (m, 1H), 3.72 (d, J=10.8 Hz, 1H), 3.21-3.11 (m, 1H), 3.10-3.01 (m, 1H), 2.00-1.92 (m, 2H), 1.38-1.22 (m, 12H), 1.19-1.16 (m, 1H).
Step 3: D6-c (110 mg, 0.27 mmol) was dissolved in acetonitrile (15 mL), 1,2-dibromoethane (2 mL) and potassium carbonate (190 mg, 1.4 mmol) were added and refluxed in an oil bath for 12 h. The mixture was filtered to remove the insoluble matter, and concentrated under reduced pressure to remove the solvent. The obtained crude product was then dissolved in acetonitrile (15 mL), 3-fluoromethyl-azetidine hydrochloride (80 mg, 0.64 mmol) and potassium carbonate (190 mg, 1.4 mmol) were added. After the mixture was refluxed in an oil bath for 2 h, the insoluble matter was filtered off, and the solvent was removed by concentration under reduced pressure. The crude product was dissolved in methanol (5 mL), sodium methoxide (0.5 mL) was added, and the mixture was stirred at room temperature for 10 min, and then concentrated under reduced pressure to remove the solvent. The mixture was neutralized with 1N HCl to pH 7-8, and extracted with dichloromethane (15 mL×3). The organic phases were combined, and then dried over anhydrous sodium sulfate, filtered, and concentrated under reduced pressure to remove the solvent to obtain a crude product, which was separated by column chromatography (dichloromethane/methanol 20:1-10:1) to obtain Example 15 (110 mg, total yield of three steps was 93%). ¹H NMR (600 MHz, CDCl₃) δ 6.94 (d, J=8.6 Hz, 2H), 6.70-6.64 (m, 3H), 6.35 (d, J=2.4 Hz, 1H), 6.27 (dd, J=8.3, 2.4 Hz, 1H), 4.51 (dd, J=47.4, 5.2 Hz, 2H), 4.20 (dd, J=10.9, 2.7 Hz, 1H), 4.07 (d, J=4.4 Hz, 1H), 4.03-3.91 (m, 4 3.87 (d, J=11.1 Hz, 1H), 3.60 (t, J=7.7 Hz, 2H), 3.37-3.30 (m, 1H), 3.27 (q, J=8.1 Hz, 2H), 3.23-3.16 (m, 1H), 3.01-2.86 (m, 3H), 2.07-1.98 (m, 2H), 1.46 (qd, J=12.8, 4.4 Hz, 1H), 1.32-1.24 (m, 2H), 1.14 (d, J=11.9 Hz, 1H).

### Example 16

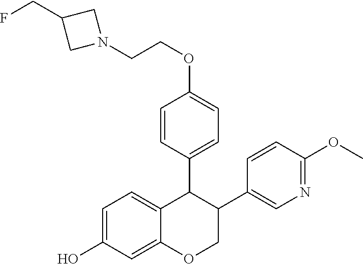 [SMILES: COc1ccc(C2COc3cc(O)ccc3C2c2ccc(OCCN3CC(CF)C3)cc2)cn1]

### Cis-4-(4-(2-(3-(fluoromethyl)azetidin-1-yl)ethoxy)phenyl)-3-(6-methoxypyridin-3-yl)-7-hydroxychroman

### Synthetic Route:

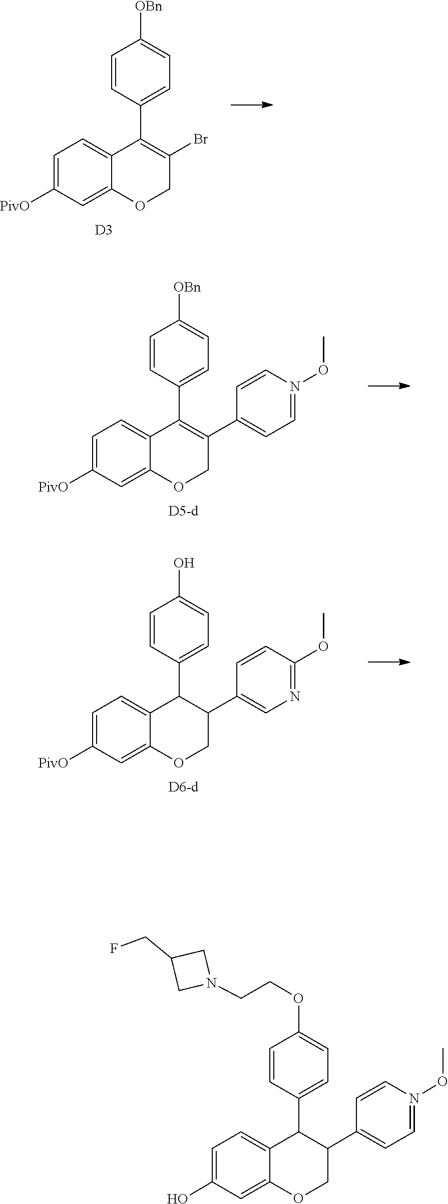

Step 1: D3 (0.6 g, 1.2 mmol), 2-methoxy-5-pyridineboronic acid (0.25 g, 1.6 mmol), Pd(PPh₃)₄ (154 mg, 0.13 mmol) and cesium carbonate (0.9 g, 2.7 mmol) were added to a reaction flask, and a mixture of 1,4-dioxane-water (4:1 v/v, 20 mL) was added, and the mixture was reacted in an oil bath at 100° C. for 4 hours under nitrogen protection. After the reaction solution was cooled, it was diluted with water (50 mL), extracted with ethyl acetate (50 mL×2), the organic phases were combined, washed with saturated sodium chloride solution (40 mL), dried over anhydrous sodium sulfate, filtered, concentrated under reduced pressure to remove the solvent, and separated by column chromatography (petroleum ether/ethyl acetate 5:1) to obtain D5-d (0.61 g, 97%). ¹H NMR (400 MHz, CDCl₃) δ 7.91 (d, J=2.3 Hz, 1H), 7.51-7.35 (m, 5H), 7.16 (dd, J=8.6, 2.5 Hz, 1H), 7.08 (d, J=8.7 Hz, 2H), 6.95 (d, J=8.7 Hz, 2H), 6.87 (d, J=8.5 Hz, 1H), 6.71 (d, J=2.1 Hz, 1H), 6.61-6.51 (m, 2H), 5.09 (s, 2H), 5.07 (s, 2H), 3.90 (s, 3H), 1.39 (s, 9H).
Step 2: D5-c (0.4 g) was dissolved in ethanol (15 mL), 20% Pd(OH)₂/C (0.4 g) was added, and the mixture was reacted under a hydrogen atmosphere (1 atm) at 60° C. for 8 h. The crude product was filtered through diatomaceous earth and concentrated under reduced pressure to remove the solvent, and then separated by column chromatography (petroleum ether/ethyl acetate 2:1) to obtain D6-d (0.14 g, 42%). ¹H NMR (400 MHz, CDCl₃) δ 7.56 (d, J=2.2 Hz, 1H), 6.95 (d, J=8.4 Hz, 1H), 6.85 (dd, J=8.6, 2.4 Hz, 1H), 6.70 (d, J=2.3 Hz, 1H), 6.60-6.47 (m, 6H), 4.38 (dd, J=10.9, 10.9 Hz, 1H), 4.25-4.17 (m, 2H), 3.90 (s, 3H), 3.52 (ddd, J=11.2, 5.3, 3.6 Hz, 1H), 1.37 (s, 9H).
Step 3: D6-d (130 mg, 0.30 mmol) was dissolved in acetonitrile (10 mL), 1,2-dibromoethane (1 mL) and potassium carbonate (210 mg, 1.5 mmol) were added and refluxed in an oil bath for 12 h. The mixture was filtered to remove the insoluble matter and concentrated under reduced pressure to remove the solvent to obtain a crude product. The crude product was then dissolved in acetonitrile (15 mL), 3-fluoromethyl-azetidine hydrochloride (90 mg, 0.72 mmol) and potassium carbonate (210 mg, 1.5 mmol) were added. After the mixture was refluxed in an oil bath for 2 h, the insoluble matter was filtered off, and the solvent was removed by concentration under reduced pressure. The crude product was dissolved in methanol (5 mL), sodium methoxide (0.5 mL) was added, and the mixture was stirred at room temperature for 10 min, and then concentrated under reduced pressure to remove the solvent. The mixture was neutralized with 1N HCl to pH 7-8, and extracted with dichloromethane (15 mL×3). The organic phases were combined, and then dried over anhydrous sodium sulfate, filtered, and concentrated under reduced pressure to remove the solvent to obtain a crude product, which was separated by column chromatography (dichloromethane/methanol 20:1-10:1) to obtain Example 16 (42 mg, total yield of three steps was 30%). ¹H NMR (400 MHz, CDCl₃) δ 7.67 (d, J=2.4 Hz, 1H), 6.67 (d, J=8.4 Hz, 1H), 6.60 (dd, J=8.6, 2.4 Hz, 1H), 6.56-6.48 (m, 5H), 6.43 (d, J=2.4 Hz, 1H), 6.33 (dd, J=8.3, 2.5 Hz, 1H), 4.52 (dd, J=47.3, 5.2 Hz, 2H), 4.29 (dd, J=11.1 Hz, 1H), 4.17-4.06 (m, 2H), 3.97-3.86 (m, 5H), 3.63 (t, J=8.3 Hz, 2H), 3.52 (ddd, J=11.5, 5.4, 3.5 Hz, 1H), 3.29 (td, J=7.5, 4.9 Hz, 2H), 3.03-2.87 (m, 3H).

### Example 17

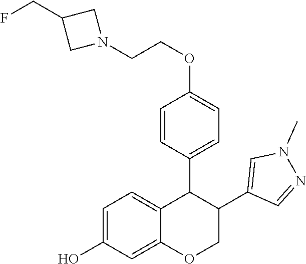 [SMILES: Cn1cc(C2COc3cc(O)ccc3C2c2ccc(OCCN3CC(CF)C3)cc2)cn1]

### Cis-4-(4-(2-(3-(fluoromethyl)azetidin-1-yl)ethoxy)phenyl)-3-(1-methyl-1H-pyrazol-4-yl)-7-hydroxychroman

### Synthetic Route:

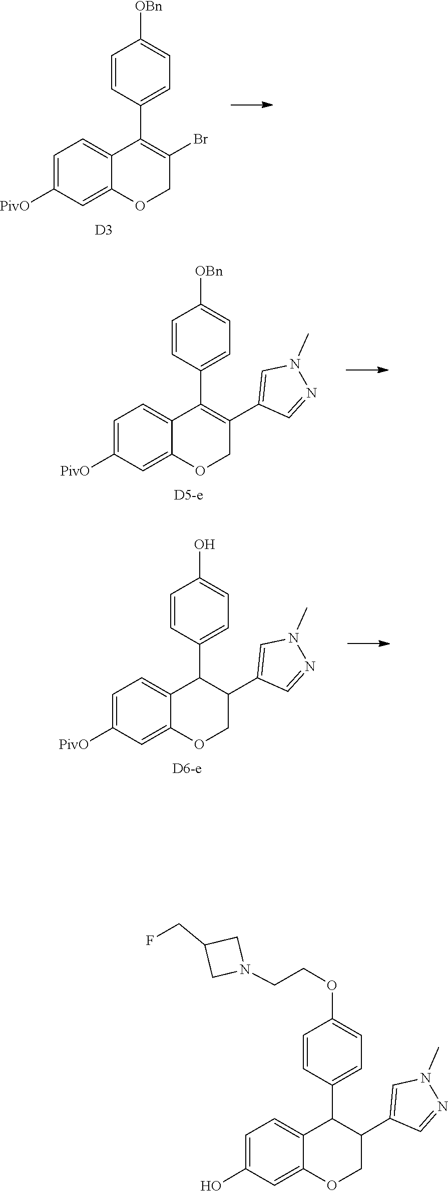

Step 1: D3 (1.0 g, 2.0 mmol), 1-methyl-1H-pyrazole-4-boronic acid (0.44 g, 0.35 mmol), Pd(PPh₃)₄ (260 mg, 0.22 mmol) and cesium carbonate (1.4 g, 4.3 mmol) were added to a reaction flask, and a mixture of 1,4-dioxane-water (4:1 v/v, 20 mL) was added, and the mixture was reacted in an oil bath at 80° C. for 4 hours under nitrogen protection. After the reaction solution was cooled, it was diluted with water (50 mL) and extracted with ethyl acetate (50 mL×2). The organic phases were combined, washed with saturated sodium chloride solution (40 mL), dried over anhydrous sodium sulfate, filtered, concentrated under reduced pressure to remove the solvent, and separated by column chromatography (petroleum ether/ethyl acetate 2:1) to obtain D5-e (0.82 g, 81%). ¹H NMR (400 MHz, CDCl₃) δ 7.53-7.35 (m, 5H), 7.18-7.09 (m, 4H), 6.97 (s, 1H), 6.71 (d, J=8.4 Hz, 1H), 6.67 (s, 1H), 6.64 (d, J=2.3 Hz, 1H), 6.51 (dd, J=8.4, 2.3 Hz, 1H), 5.15 (s, 2H), 5.10 (s, 2H), 3.75 (s, 2H), 1.36 (s, 9H).
Step 2: D5-c (0.8 g) was dissolved in ethanol (15 mL), and 20% Pd(OH)₂/C (0.8 g) was added, and the mixture was reacted under a hydrogen atmosphere (1 atm) at 60° C. for 8 h. The crude product was filtered through diatomaceous earth and concentrated under reduced pressure to remove the solvent, and then separated by column chromatography (petroleum ether/ethyl acetate 2:1) to obtain D6-e (0.57 g, 86%). ¹H NMR (400 MHz, CDCl₃) δ 6.99 (s, 1H), 6.93 (d, J=8.4 Hz, 1H), 6.70 (s, 1H), 6.68 (d, J=2.3 Hz, 1H), 6.64-6.54 (m, 5H), 4.35-4.22 (m, 3H), 3.80 (s, 3H), 3.49-3.40 (m, 1H), 1.37 (s, 9H).
Step 3: D6-e (220 mg, 0.54 mmol) was dissolved in acetonitrile (10 mL), 1,2-dibromoethane (2 mL) and potassium carbonate (373 mg, 2.7 mmol) were added and refluxed in an oil bath for 12 h The mixture was filtered to remove the insoluble matter and concentrated under reduced pressure to remove the solvent to obtain a crude product. The crude product was then dissolved in acetonitrile (15 mL), 3-fluoromethyl-azetidine hydrochloride (140 mg, 1.12 mmol) and potassium carbonate (373 mg, 2.7 mmol) were added. After the mixture was refluxed in an oil bath for 4 h, the insoluble matter was filtered off, and the solvent was removed by concentration under reduced pressure. The crude product was dissolved in methanol (5 mL), sodium methoxide (0.5 mL) was added, and the mixture was stirred at room temperature for 10 min, and then concentrated under reduced pressure to remove the solvent. The mixture was neutralized with 1N HCl to pH 7-8, and extracted with dichloromethane (15 mL×3). The organic phases were combined, and then dried over anhydrous sodium sulfate, filtered, and concentrated under reduced pressure to remove the solvent to obtain a crude product, which was separated by column chromatography (dichloromethane/methanol 25:1-10:1) to obtain Example 17 (119 mg, total yield of three steps was 50%). ¹H NMR (600 MHz, CDCl₃) δ 6.92 (s, 1H), 6.68-6.62 (m, 3H), 6.60-6.54 (m, 3H), 6.40 (d, J=2.4 Hz, 1H), 6.30 (dd, J=8.3, 2.4 Hz, 1H), 4.51 (dd, J=47.4, 5.3 Hz, 2H), 4.19-4.08 (m, 3H), 3.97-3.88 (m, 2H), 3.75 (s, 3H), 3.60 (t, J=7.5 Hz, 2H), 3.46 (ddd, J=9.6, 5.4, 3.8 Hz, 1H), 3.29-3.24 (m, 2H), 2.97-2.86 (m, 3H).

### Example 18

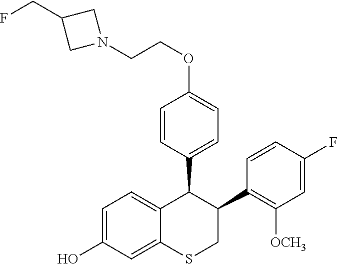 [SMILES: COc1cc(F)ccc1[C@H]1CSc2cc(O)ccc2[C@H]1c1ccc(OCCN2CC(CF)C2)cc1]

### (3S,4R)-3-(4-fluoro-2-methoxyphenyl)-4-(4-(2-(3-(fluoromethyl)azetidin-1-yl)ethoxy)phenyl-)7-hydroxythiochroman

The compound shown in Example 18 was obtained by chiral preparative HPLC separation of Example 4, and the separation conditions were: semi-preparative CHIRALCEL OD-H chiral chromatographic column (length 250 mm, inner diameter 20 mm, filler particle size 5 μm), and the mobile phase was isopropanol:n-hexane (4:6). The NMR data of Example 18 was consistent with that of Example 4, and ee>99%.

### Example 19

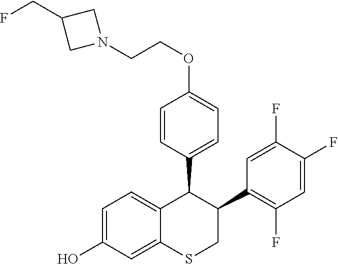 [SMILES: Oc1ccc2c(c1)SC[C@H](c1cc(F)c(F)cc1F)[C@@H]2c1ccc(OCCN2CC(CF)C2)cc1]

### (3S,4R)-4-(4-(2-(3-(fluoromethyl)azetidin-1-yl)ethoxy)phenyl)-3-(2,4,5-trifluorophenyl)-7-hydroxythiochroman

The compound shown in Example 19 was obtained by chiral preparative HPLC separation of Example 5, and the separation conditions were: semi-preparative CHIRALCEL OD-H chiral chromatographic column (length 250 mm, inner diameter 20 mm, filler particle size 5 m), and the mobile phase was isopropanol:n-hexane (4:6). The NMR data of Example 19 was consistent with that of Example 5, and ee>99%.

### Example 20

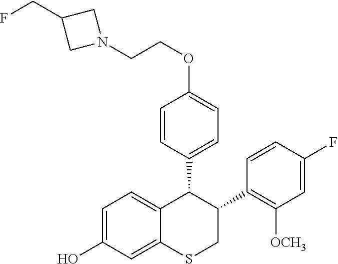 [SMILES: COc1cc(F)ccc1[C@@H]1CSc2cc(O)ccc2[C@@H]1c1ccc(OCCN2CC(CF)C2)cc1]

### (3R,4S)-3-(4-fluoro-2-methoxyphenyl)-4-(4-(2-(3-(fluoromethyl)azetidin-1-yl)ethoxy)phenyl-)7-hydroxythiochroman

The compound shown in Example 20 was obtained by chiral preparative HPLC separation of Example 4, and the separation conditions were: semi-preparative CHIRALCEL OD-H chiral chromatographic column (length 250 mm, inner diameter 20 mm, filler particle size 5 μm), and the mobile phase was isopropanol:n-hexane (4:6). The NMR data of Example 20 was consistent with that of Example 4, and ee>99%.

### Example 21

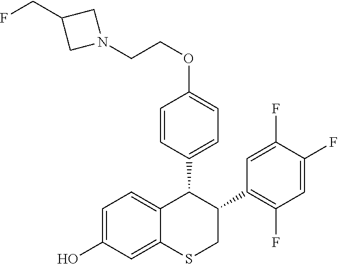 [SMILES: Oc1ccc2c(c1)SC[C@@H](c1cc(F)c(F)cc1F)[C@H]2c1ccc(OCCN2CC(CF)C2)cc1]

### (3R,4S)-4-(4-(2-(3-(fluoromethyl)azetidin-1-yl)ethoxy)phenyl)-3-(2,4,5-trifluorophenyl)-7-hydroxythiochroman

A8-e was separated by chiral preparative HPLC to obtain the compound shown in Example 21. The separation conditions were: semi-preparative CHIRALCEL OD-H chiral chromatographic column (length 250 mm, inner diameter 20 mm, filler particle size 5 μm), mobile phase isopropanol:n-hexane (4:6). The NMR data of Example 21 were consistent with those of Example 5, ee>99%.

### Example 22

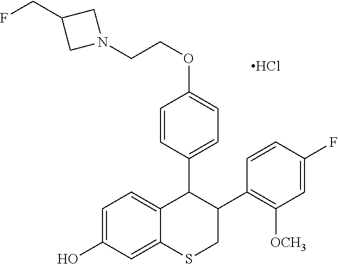 [SMILES: COc1cc(F)ccc1C1CSc2cc(O)ccc2C1c1ccc(OCCN2CC(CF)C2)cc1 ; Cl]

### Cis-3-(4-fluoro-2-methoxyphenyl)-4-(4-(2-(3-(fluoromethyl)azetidin-1-yl)ethoxy)phenyl)-7-hydroxythiochroman Hydrochloride

Cis-3-(4-fluoro-2-methoxyphenyl)-4-(4-(2-(3-(fluoromethyl)azetidin-1-yl)ethoxy)phenyl)-7-hydroxythiochroman (50 mg, 0.1 mmol) was dissolved in 1,4-dioxane (5 mL), followed by addition of a solution of hydrogen chloride in 1,4-dioxane (4 M, 40 μM), and stirred at room temperature for 1 hour. The reaction solution was concentrated to remove the solvent, and then methyl tert-butyl ether (2 mL) was added and stirred at room temperature for 30 minutes, filtered, and the filter cake was drained to obtain a white powder (45 mg, 84%). ¹H NMR (400 MHz, DMSO-d₆) δ 11.16 (s, 1H), 9.46 (s, 1H), 6.96 (d, J=10.7 Hz, 1H), 6.72 (t, J=8.1 Hz, 3H), 6.62 (s, 1H), 6.57-6.48 (m, 1H), 6.47-6.29 (m, 3H), 6.20 (t, J=7.4 Hz, 1H), 4.83-4.42 (m, 2H), 4.25-4.08 (m, 4H), 4.03-3.81 (m, 5H), 3.72 (d, J=12.9 Hz, 1H), 3.54-3.44 (m, 1H), 3.41-3.19 (m, 3H), 3.16-3.03 (m, 1H), 2.76 (d, J=12.0 Hz, 1H).

### Example 23

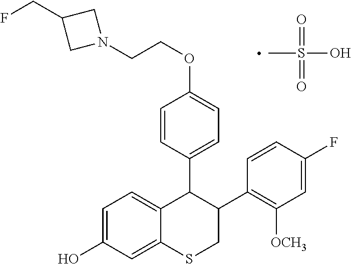

### Cis-3-(4-fluoro-2-methoxyphenyl)-4-(4-(2-(3-(fluoromethyl)azetidin-1-yl)ethoxy)phenyl)-7-hydroxythiochroman Methanesulfonate

Cis-3-(4-fluoro-2-methoxyphenyl)-4-(4-(2-(3-(fluoromethyl)azetidin-1-yl)ethoxy)phenyl)-7-hydroxythiochroman (50 mg, 0.1 mmol) was dissolved in acetonitrile (5 mL), followed by the addition of methanesulfonic acid (9.7 mg, 0.1 mmol) and stirred at room temperature for 1 hour. After the addition of water (10 mL), the mixture was freeze-dried to obtain a white foamy solid (55 mg, 94%). ¹H NMR (400 MHz, CD3 OD) δ 6.87-6.81 (m, 1H), 6.76-6.67 (m, 3H), 6.63 (d, J=2.5 Hz, 1H), 6.49 (d, J=8.2 Hz, 2H), 6.46-6.34 (m, 2H), 6.18 (t, J=7.6 Hz, 1H), 4.74-4.56 (m, 2H), 4.51-4.24 (m, 5H), 4.20-4.10 (m, 3H), 3.95-3.81 (m, 4H), 3.70-3.64 (m, 1H), 3.61-3.55 (m, 1H), 3.43-3.35 (m, 1H), 2.72 (s, 3H), 2.70-2.61 (m, 1H).

### Biological Evaluation

The present invention is further described below in conjunction with test examples, but these examples are not intended to limit the scope of the present invention.

### Test Example 1: Evaluation of the Inhibitory Activity of the Compounds of the Present Invention on the Proliferation of Breast Cancer MCF-7 Cells

The purpose of this experiment is to determine the in vitro proliferation inhibitory effect of the compounds of the present invention on the commonly used breast cancer estrogen receptor-positive cells, MCF-7 cells, and to evaluate the in vitro activity of the compounds based on IC₅₀.

The inhibitory effect of the compounds of the present invention on the proliferation of MCF-7 cells was tested by the ATP method, specifically the CTG method. CellTiter-Glo, referred to as CTG, is a rapid homogenous detection method for determining the number of viable cells in culture by quantifying ATP. ATP is a key indicator of the metabolism of living cells. The homogenous detection makes the cell lysis and the generated luminescent signal proportional to the amount of ATP present, and the amount of ATP is directly proportional to the number of cells in the culture. The specific experimental method is as follows.

MCF-7 cells were cultured in DMEM+10% FBS. The cells were cultured in a 37° C. incubator under 5% CO₂. Cell passaging, recovery and cryopreservation were performed according to conventional methods. When the growth reached about 80% polymerization, MCF-7 cells were digested by trypsin and inoculated into 96-well plates at 4×10³ cells per well, with an inoculation volume of 90 μL per well, and cultured in a 37° C. incubator overnight. On the second day, the drug with concentration gradient was added, 10 μL per well. The highest detectable concentration of the compound was 10 μM, 4-fold dilution, 10 concentrations. The 96-well plate was incubated in 5% CO₂ at 37° C. for 6 days. 50 μL of CTG reagent was added to each well of the 96-well plate, and the plate was shaken on a shaker for 5 min, and then left at room temperature and protected from light for 10 min. 60 μL of CTG reagent was transferred to the 384-well Opti-plate. The luminescence signals were read by a multifunctional enzyme marker, and the signal intensity was used to characterize the number of viable cells. The data of each dosing group were normalized to 100% of the mean value of the control group, and the data were processed and analyzed by GraphPad Prism software. The results are shown in Table 1.

TABLE 1
Inhibitory activity of the compounds of the
present invention on MCF-7 cell proliferation

| Compound | IC₅₀ (nM) | Compound | IC₅₀ (nM) |
|---|---|---|---|
| Fulvestrant | 1.9 | Example 10 | 1.7 |
| SAR439859 | 3.9 | Example 7 | 0.88 |
| Example 1 | 0.65 | Example 8 | 1.6 |
| Example 2 | 0.77 | Example 15 | 1.6 |
| Example 3 | 0.42 | Example 16 | 0.86 |
| Example 4 | 0.53 | Example 18 | 0.25 |
| Example 5 | 0.45 | Example 19 | 0.25 |
| Example 6 | 0.68 | | |

As can be seen from Table 1, the compounds of the present invention have a strong inhibitory effect on the proliferation of MCF-7 breast cancer cells, especially the compounds of Examples 18 and 19, whose anti-tumor activity is 7.6 times higher than that of the positive control fulvestrant and 15.6 times higher than that of SAR439859, reaching the sub-nanomolar level.

### Test Example 2: Evaluation of the Inhibitory Activity of the Compounds of the Present Invention on Tamoxifen-Resistant MCF-7 Tam1 Cells

MCF-7 Tam1 cells are established by long-term exposure of MCF-7 cells to 4-hydroxytamoxifen, the active metabolite of tamoxifen, accompanied by long-term estrogen deprivation culture conditions, and have resistance to tamoxifen and aromatase inhibitors. In this experiment, the proliferation inhibition of the compounds of the present invention on tamoxifen-resistant cells MCF-7 Tam1 was detected to evaluate the in vitro anti-tamoxifen-resistant breast cancer activity of the compounds based on the IC₅₀ value.

MCF-7 Tam1 cells were subcultured in DMEM medium containing penicillin (final concentration of 100 U/mL), streptomycin (final concentration of 100 μg/mL), 10 μg/mL human insulin, 1 μM 4-hydroxytamoxifen and 10% FBS. When the cells were fused to 90%, the old medium was discarded and the cells were washed twice with 2 ml PBS. After discarding PBS, 2 mL 0.25% trypsin-0.02% EDTA mixed digestion solution was added and observed under a microscope for about 30 s. When the cells became round, 2 ml complete medium was quickly added to terminate the digestion. The cells were gently blown, collected and centrifuged at 800 rpm, 4° C., for 5 min, the supernatant was discarded, the cells were resuspended with complete medium, cultured in bottles, and the medium was changed every other day. The highest detection concentration of the compound was 10 μM, 10-fold dilution, 8 concentrations. A 96-well plate suitable for chemiluminescence detection was used, and 100 μl cells (1×10⁴ cells/ml) were inoculated in each well. Each group of cells was incubated with medium containing the corresponding concentration of compound for 5 days according to the experimental group. The cell culture plate was taken out and equilibrated at room temperature for 10 minutes. 100 μl CTG detection reagent was added to each well of the 96-well plate and incubated at room temperature for 10 minutes. The RFU value of each group was detected with an ELISA instrument. The inhibition rate was calculated as follows: inhibition rate=(OD value of the control group−OD value of the drug group)/OD value of the control group. The IC₅₀ of each compound on MCF-7Tam1 cells was calculated by using GraphPad.

TABLE 2
Inhibitory activity of the compounds of the present
invention on tamoxifen-resistant cells MCF-7 Tam1

| Compound | IC₅₀ (nM) |
|---|---|
| Fulvestrant | 3.32 ± 0.53 |
| SAR439859 | 17.63 ± 0.85 |
| Example 4 | 6.84 ± 1.4 |
| Example 18 | 5.37 ± 0.42 |
| Example 19 | 6.30 ± 0.92 |

Table 2 shows that the compound of the present invention has a strong inhibitory effect on the proliferation of tamoxifen-resistant breast cancer cells MCF-7 Tam1, and its anti-tumor activity is similar to that of fulvestrant and significantly better than that of SAR439859.

### Test Example 3: Evaluation of the Degradation Activity of the Compounds of the Present Invention on Wild-Type ERα in MCF-7 Cells

The extent of ER degradation induced by the compounds of the present invention was analyzed by a cell-based high-content imaging method.

MCF-7 cell culture medium: 88% RPMI 1640, 10% FBS, 1% P/S and 1% GlutaMax; MCF-7 seeding medium: 88% RPMI 1640, 10% FBS, 1% P/S and 1% GlutaMax.

Day 1: Dilute cell suspension to 8.75×10⁴ cells/mL with cell seeding medium, then drop 40 μL of cell suspension into each well of the assay plate and place in a 37° C. CO₂ incubator. Day 3: Dilute compound 4-fold serial dilutions, transfer 500 μL, 10 concentration points. Dilute compound with 20 μL medium, transfer 10 μL to plate with Bravo, and incubate in a 37° C. incubator for 24 hours. Day 4: Add 50 μL of 8% paraformaldehyde to the assay plate and incubate at room temperature for 40 minutes. Wash the assay plate twice with 100 μL PBS per well, then add 50 μL of 0.1% TritonX-100 in PBS to the assay plate and incubate at room temperature for 15 minutes. Wash the assay plate 5 times with 100 μL PBS per well, then add 50 μL of blocking buffer containing 0.1% Tween 20 and incubate at room temperature for 1 hour. Discard the solution in the test plate, dilute the primary antibody with blocking buffer at a ratio of 1:1000, add 25 μL to the test plate, and incubate at 4° C. overnight. Day 5: Wash the test plate 5 times with 100 μL PBS per well, then dilute the secondary antibody (1:1000) and DRAQ5 (1:2000) with blocking buffer, add 25 μL to the test plate, and incubate at room temperature for 1 hour. Wash the test plate 5 times with 100 μL PBS per well and read the test plate on the Odyssey infrared imaging system. The data were then normalized by dividing the integrated intensity of the 800 channel (ER) by the integrated intensity of the 700 channel (DNA). Background subtraction was performed by subtracting the average of the normalized negative control wells (no primary antibody) from all normalized experimental values. The percentage response of the normalized/background subtracted data was then calculated by dividing each experimental value by the average of the DMSO control values (% response=(experimental value/DMSO control value)×100). Dose response curves, EC₅₀, and percent ERα remaining values were generated using GraphPad Prism v6.02 software (GraphPad, San Diego, CA).

TABLE 3
Degradation activity of the compounds of the present
invention on wild-type ERα in MCF-7 cells

| Compound | IC₅₀ (nM) |
|---|---|
| Fulvestrant | 1.1 |
| SAR439859 | 1.7 |
| Example 1 | 0.62 |
| Example 2 | 1.0 |
| Example 3 | 0.57 |
| Example 4 | 1.0 |
| Example 5 | 1.1 |
| Example 6 | 1.1 |
| Example 7 | 1.4 |
| Example 8 | 3.1 |
| Example 15 | 2.5 |
| Example 16 | 2.0 |
| Example 18 | 0.43 |
| Example 19 | 0.46 |

As can be seen from Table 3, the compounds of the present invention have a strong degradation effect on ERα, especially the compounds of Examples 1, 3, 18 and 19, whose ER degradation activity is about 2 times higher than that of the positive control fulvestrant and about 3 times higher than that of SAR439859, reaching the subnanomolar level.

### Test Example 4: The Antagonistic Activity of Compounds Against Wild-Type ERα to Detected by MCF-7 Cell Fluorescence Reporter Gene Assay

Principle of ERα reporter gene construction: HEK293/GAL4/ERα, the cell line uses molecular cloning methods to express the fusion protein of the estrogen receptor LBD region (compound binding region) and the GAL4 DBD region (DNA binding region). When the ligand activates ERα, the ERα-GAL4 fusion protein activates the expression of the downstream luciferase gene, and the plate reader detects the chemiluminescent signal. The ERα ligand stimulation concentration and the chemiluminescent signal are dose-dependent.

The HEK293/GAL4/ERα cell suspension was collected and centrifuged at 1000 rpm for 5 minutes. The supernatant was removed and the cells were resuspended with preheated culture medium (DMEM (phenol red-free), containing 10% carbon-adsorbed serum, i.e., 450 mL DMEM and 50 mL carbon-adsorbed serum in 500 mL cell culture medium. After counting, the cell suspension was diluted with culture medium and inoculated into a 96-well cell culture plate at 40,000 cells/well. 80 μL of cell suspension was inoculated into each well and incubated overnight at 37° C., 5% CO₂ incubator. On the day of the experiment, 10 μL of compound working solution was added to each well of the cell plate, and the incubation continued for 1 hour at 37° C., 5% CO₂ incubator. Then, 10 μL of culture medium containing agonist (10 nM Estradiol) was added to each well. The final concentration of Estradiol was 1 nM and the final concentration of DMSO was 0.5%. The cell plate was incubated for 24 hours at 37° C. and 5% CO₂ incubator. After the incubation, the cell supernatant was removed, and 50 μL Bright Glo detection reagent was added to each well of the cell plate, incubated at 25° C. for 2 minutes. After the incubation, EnVision was used to detect the luminescent signal.

The data were calculated according to the following formula to calculate the inhibition rate after compound treatment: % inhibition rate=100−(RFU compound−RFU blank control)/(RFU negative control−RFU blank control)×100%. Negative control: cells treated with agonists; blank control: cells not treated with agonists, and then Prism was used to plot and calculate the IC₅₀ value of the compound.

TABLE 4
In vitro antagonistic activity of representative compounds
of the present invention against wild-type ERα

| Compound | IC₅₀ (nM) |
|---|---|
| Fulvestrant | 1.3 |
| SAR439859 | 2.0 |
| Example 1 | 0.97 |
| Example 2 | 0.74 |
| Example 3 | 0.55 |
| Example 4 | 0.64 |
| Example 5 | 0.61 |
| Example 6 | 0.85 |
| Example 7 | 0.88 |
| Example 15 | 1.3 |
| Example 16 | 1.3 |
| Example 18 | 0.56 |
| Example 19 | 0.51 |

It can be seen from the experimental results in Table 4 that the compounds of the present invention have a strong antagonistic effect on wild-type ERα, which is superior to fulvestrant and clinical phase III drug SAR439859. Among them, the antagonistic activity of the compounds of Examples 3, 4, 5, 18, and 19 on wild-type ERα is about 3 times higher than that of SAR439859, and more than 2 times higher than that of fulvestrant.

### Test Example 5: Antagonistic Effect of the Compounds of the Present Invention on Mutant ERα Containing Y537S or D538G Mutations

15%-30% of breast cancer patients will have mutations in the ESR1 ligand binding region after receiving endocrine therapy, with D538G and Y537S mutations being the most common, especially in patients with estrogen receptor-positive metastatic breast cancer, and mutations are associated with poor treatment effects. Unlike wild-type ERα, Y537S and D538G mutations in the ligand binding domain of ERα lead to spontaneous recruitment of coactivators, such as peroxisome proliferator-activated receptor-γ coactivators and steroid receptor coactivators, in the absence of ligands, leading to constitutive activation of ERα and promoting the formation of an AF-2 agonist-like conformation. In this activated conformation, the mutant ERα has an increased affinity for estradiol and a reduced affinity for antagonists. This experiment evaluates the in vitro antitumor activity of the compounds of the present invention against endocrine therapy-resistant breast cancer by detecting the antagonistic activity of the compounds against mutant ERα containing the main mutation Y537S and D538G.

SK-BR-3 Cell Culture Medium: 89% 1640 without phenol red, 10% charcoal-treated FBS and 1% GlutaMax

Day 1: 1. Inoculate 80 μL of cell suspension and 30,000 cells into each well of the assay plate and incubate at 37° C. 5% CO₂ for 24 hours. Day 2: Prepare the transfection reagent and leave at room temperature for 15 minutes. Add 10 μL of transfection reagent to each well of the assay plate and incubate at 37° C. 5% CO₂ for 24 hours. Day 3: Add 10 μL of culture medium (100 nM β-estradiol, 10 μL of culture medium) to the assay plate and incubate at 37° C. 5% CO₂ for 24 hours. Day 4: 1. Remove 50 μL of culture medium from each well, add 50 μL of luciferase assay reagent to the assay plate, shake at room temperature for 20 minutes, and then read on Envision. 2. Add 50 μL of Stop&Glo reagent to the assay plate, shake at room temperature for 20 minutes, and then read on Envision. The data were analyzed using XL-fit software (supplier: ID Business Solutions Ltd., software version: XL fit 5.0), % Effect=(sample value−blank control)/(positive control−blank control)*100.

TABLE 5
In vitro antagonistic activity of the compounds
of the present invention against mutant ERα

Erα mutant

| Compound | Y537S (IC₅₀/nM) | D538G (IC₅₀/nM) |
|---|---|---|
| Fulvestrant | 15.3 | 13.3 |
| SAR439859 | 17.2 | 57.7 |
| Example 4 | 11.1 | 21.7 |
| Example 18 | 4.6 | 10.1 |
| Example 19 | 3.8 | 11.0 |

The data in Table 5 show that the compounds of the present invention have a strong antagonistic effect on mutant ERα (Y537S or D538G). Among them, for ERα mutants containing Y537S mutation, Example 4 has comparable antagonistic activity to Fulvestrant and SAR439859, Example 18 has 3.3-fold and 3.7-fold improvement over Fulvestrant and SAR439859, respectively, and Example 19 has 4-fold and 4.5-fold improvement over Fulvestrant and SAR439859, respectively; for ERα mutants containing D538G mutation, Examples 18 and 19 have similar antagonistic activity to Fulvestrant, but have 5.7-fold and 5.2-fold improvement over SAR439859, respectively.

The results of test examples 1 to 5 show that the compounds of the present invention have significant dual functions of antagonism/degradation of ERα, including significant antagonism of mutant ERα containing common mutation points, and significant antiproliferative activity against estrogen-dependent MCF-7 cells and tamoxifen-resistant MCF-7 Tam1 cells. Based on the results of these tests, it can be seen that the compounds of the present invention exhibit excellent in vitro antitumor efficacy, and are superior to the marketed drug Fulvestrant in multiple tests, and are significantly superior to the control compound SAR439859.

### Test Example 6: Evaluation of the Pharmacokinetic Properties of the Compounds of the Present Invention in Rats

The purpose of this experiment is to test the pharmacokinetic properties of the compounds of the present invention in rats.

The solvent is 5% DMSO+5% Solutol+90% (0.5% MC), which is a colorless and clear dosing solution. The dosing method and dosage are: oral, 10 mg/kg. SD rats were randomly divided into groups according to body weight, fasted but not watered for 12-14 hours one day before dosing, and fed 4 hours after dosing. 0.1 mL of blood was collected from each animal through the eye socket each time, anticoagulated with EDTAK2, and the collection time points were: 0, 5, 15, 30 minutes, 1, 2, 4, 6, 8, 24 hours after administration of the test compound. After blood sample collection, it was placed on ice and centrifuged within 30 minutes to separate plasma (centrifugation conditions: 5000 rpm, 10 minutes, 4° C.). It was stored at −80° C. before analysis. The concentration of compounds in rat plasma was determined by LC-MS/MS. The data acquisition and control system software was Analyst 1.5.1 software (Applied Biosystem). The peak integration method of the chromatogram sample was automatic integration; the ratio of the sample peak area to the internal standard peak area was used as an indicator and regressed with the concentration of the sample. Regression method: linear regression, weight coefficient is 1/X². Pharmacokinetic parameters were analyzed and processed by WinNonlin Professional v6.3 (Pharsight, USA) using non-compartmental model. Cₘₐₓ is the maximum measured blood drug concentration, the area under the blood drug concentration-time curve AUC (0→t) was calculated by the trapezoidal method, and Tₘₐₓ is the time when the blood drug concentration reaches the peak after administration. Experimental data are expressed as “mean±standard deviation” (Mean±SD, n≥3) or “mean” (Mean, n=2).

TABLE 6
Pharmacokinetic parameters of compounds of
Examples 18 and 19 of the present invention

| | T_{1/2} | Tₘₐₓ | Cₘₐₓ | AUC₀₋ₜ | MRT_{PO} |
|---|---|---|---|---|---|
| Compound | (h) | (h) | (ng/mL) | (h · ng · mL⁻¹) | (h) |
| Example 18 | 8.92 | 3.00 | 198 | 1365 | 13.4 |
| Example 19 | 4.61 | 4.00 | 197 | 1843 | 8.14 |

Table 6 shows that the compounds of the present invention have good oral absorption and good drug exposure, indicating that the compounds can be orally administered in animals for efficacy and clinical application.

### Test Example 7: Evaluation of the Effect of the Compound on Rat Uterine Weight Gain and Tissue Distribution

Estrogen receptor modulators (SERMs) such as tamoxifen in endocrine therapy for ER-positive breast cancer increase the risk of endometrial hyperplasia, polyps, and endometrial cancer due to their partial agonist effects. Therefore, when developing selective estrogen receptor degraders (SERDs), this effect needs to be eliminated to make them complete ER antagonists. By examining the effects of compounds on rat uterine weight (expressed as uterine wet weight/rat body weight) and endometrium, it can be determined whether the compound is a complete antagonist, which is of great significance for safety evaluation. On the other hand, the ability of drugs to penetrate the blood-brain barrier of animals and have sufficient exposure in brain tissue is the key to their effectiveness against brain metastases. Therefore, by analyzing the distribution of drugs in animal plasma and brain after administration, it can be determined whether the drug has the potential to have an anti-tumor effect in the brain in situ tumor model.

The uterine wet weight experiment and the tissue distribution experiment of the compound involved in this test were carried out in the same batch of rats.

Experimental animals: SPF female immature SD rats, 21 days old, provided by Changzhou Cavens Laboratory Animal Co., Ltd., raised in an SPF-grade breeding environment, with the indoor temperature controlled at 23±2° C., free access to food and water. A total of 30 animals. Adaptive breeding for 3 days before the experiment.

Experimental groups: Blank group: 2% Tween-80/0.5% hydroxymethylcellulose (10 mg/kg) was taken orally every day for 3 consecutive days. Blank+17α-ethinyl estradiol group: 17α-ethinyl estradiol (0.1 mg/kg) was taken orally every day for 3 consecutive days. 4-Hydroxytamoxifen group: 4-Hydroxytamoxifen (60 mg/kg) was taken orally every day for 3 consecutive days. Example 18 group: Example 18 (10 mg/kg) was taken orally every day for 3 consecutive days. Example 19 group: Example 19 (10 mg/kg) was taken orally every day for 3 consecutive days.

4 hours after the last administration, the rats were killed by carbon dioxide method, the uterus was dissected, irrelevant tissues were carefully removed, washed 2-3 times with D-Hanks solution to remove blood, drained and stored, and weighed. HE staining was used to detect the thickness of the endometrium. Plasma and brain tissue were sampled and the concentrations of Example 18 and Example 19 in the plasma and brain tissue were analyzed.

HE experiment: 1) Cut the fixed uterine tissue into 4 m thick slices and place the slices in an oven for 1 hour; 2) Dewax the dried paraffin slices with conventional xylene, hydrate with gradient ethanol, and wash with distilled water; 3) Add hematoxylin to stain for 10 min to 30 min, then wash away the hematoxylin stain with running water; 4) Decolorize with 1% hydrochloric acid ethanol until the slices turn red and lighter in color, and then put them in running water to restore them to blue. 5) Stain with eosin for 1 min and rinse with running water; 6) Dehydrate and dry the slices with gradient alcohol, make them transparent with xylene, and seal them with neutral gum. 7) Randomly select a field of view and take pictures with a microscope (200×). The statistical differences between the data of groups were analyzed using one-way ANOVA and Tukey's test, and P values less than 0.05 were considered significant.

LC-MS/MS detection of drug concentration: (1) Chromatographic conditions, chromatographic column: Waters BEH C18 Liquid Chromatography column (50 mm×2.1 mm, id 1.7 μm), column temperature 40° C.; mobile phase A: ultrapure water (containing 0.1% formic acid), mobile phase B: methanol. Elution gradient: 0-1 min, A-B (70:30), 1-5 min, A-B (10:90), 5-6 min, A-B (10:90), 6-6.1 min, A-B (70:30), 6.1-9 min, A-B (70:30). The elution time is 9 min, and the flow rate is 0.3 mL/min. (2) Mass spectrometry conditions: LC-MS/MS was used for determination, the ion source was ESI source, positive ion mode detection; the heating capillary temperature was 450° C.; CAD was 4; the curtain gas was 11; GS1 (N2) was 40; GS2 (N2) was 40; the scanning mode was multi-stage reaction monitoring (MRM). The ion reactions used for quantitative analysis were m/z 498.80→139.30 (Example 18) and m/z 504.70→116.20 (Example 19). (3) Standard curve: Accurately weigh the standard samples of Example 18 and Example 19, prepare a standard stock solution with a concentration of 1 mg/mL, dilute it to a standard working solution with a concentration of 10, 20, 50, 100, 200, 500, 1000, 2000, 5000, 10000, 20000, 50000 ng/mL, add 5 μL of each concentration of standard working solution to 45 μL of blank matrix, and prepare standard samples with a final concentration of 1, 2, 5, 10, 20, 50, 100, 200, 500, 1000, 2000, 5000 ng/mL, vortex mix for 3 min, add 150 μL of methanol containing internal standard (4-chlorophenylalanine), vortex oscillation for 3 min, centrifuge at 18000 rpm for 5 min, take 150 μL of supernatant and transfer it to a new Eppendorf tube, centrifuge at 18000 rpm for 5 min, take 100 μL of supernatant and transfer it to a sample injection bottle, and inject 5 μL. (4) Concentration determination of Examples 18 and 19 in samples: Take 50 μL of the plasma sample to be tested into an Eppendorf tube, vortex mix for 3 min, add 150 μL of methanol containing internal standard (4-chlorophenylalanine), vortex oscillation for 3 min, centrifuge at 18000 rpm for 5 min, take 150 μL of supernatant and transfer it to a new Eppendorf tube, centrifuge at 18000 rpm for 5 min, take 100 μL of supernatant and transfer it to a sample injection bottle, and inject 5 μL for analysis.

The results of the uterine wet weight test are as follows.

TABLE 7
Effects of representative compounds of the present
invention on uterine weight of immature rats

| | Blank | Blank + 17α- | 4-hydroxy- | Exam- | Exam- |
|---|---|---|---|---|---|
| Group | group | ethinylestradiol | tamoxifen | ple 18 | ple 19 |
| Mean body | 42.9 | 42.4 | 43.9 | 43 | 44.6 |
| weight (g) | | | | | |
| Mean uterine | 29 | 86.4 | 61.8 | 16.8 | 17.6 |
| wet weight | | | | | |
| (mg) | | | | | |
| Uterine wet | 0.068 | 0.204 | 0.14 | 0.04 | 0.038 |
| weight: body | | | | | |
| weight | | | | | |

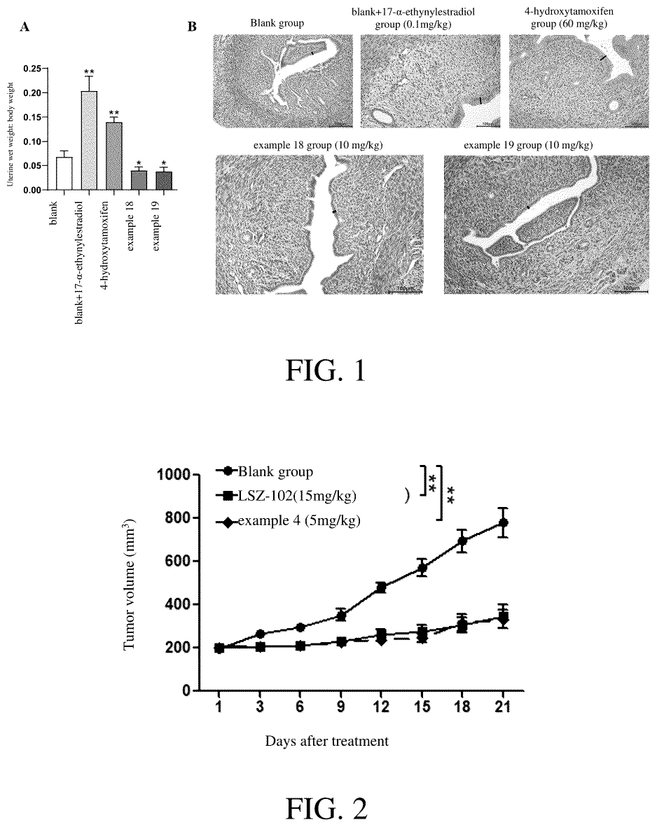

The experimental results are shown in Table 7 and FIG. 1. The ratio of uterine weight to body weight in the 17α-ethinyl estradiol group and the 4-hydroxytamoxifen (the active metabolic form of tamoxifen in vivo) group was 3 times and 2 times that of the blank group, indicating an agonistic effect on ERα of uterine tissue. However, Examples 18 and 19 of the present invention both showed an effective effect of reducing uterine weight, reducing the ratio of uterine wet weight to body weight to about 60% of that of the blank group, indicating an antagonistic/inverse agonistic effect on ERα. In addition, the endometrium was stained with hematoxylin and eosin (FIG. 1, B) for histological evaluation. 17α-ethinyl estradiol and 4-hydroxytamoxifen increased uterine wet weight, and epithelial cells showed a high columnar phenotype, while the epithelial cells of the blank control and Example 18 and Example 19 groups showed a low cubic phenotype, confirming that the example compound of the present invention is a complete antagonist and does not have the risk of endometrial cancer with estrogen receptor modulators such as tamoxifen.

TABLE 8
Tissue distribution of representative compounds of
the present invention in rats (PO-10 mg/kg, QD)

| Test | Concentration of Drug | Concentration of Drug | B/P |
|---|---|---|---|
| compound | in Plasma (ng/mL) | in Brain (ng/g) | ratio |
| Example 18 | 1230 | 17400 | 14.1 |
| Example 19 | 77.8 | 2258 | 29 |

As shown in Table 8, the compounds of the present invention exhibit excellent blood-brain barrier penetration ability and high drug exposure in the brain tissue of rats, among which Example 18 has very good brain tissue exposure, with a B/P value of 14.1, which is much higher than other estrogen degraders disclosed so far, and the B/P value of Example 19 is as high as 29. These results indicate that the compounds of the present invention can be used to treat brain metastatic breast cancer.

### Test Example 8: Growth Inhibition Experiment of the Compound of the Present Invention on MCF-7 Mouse Subcutaneous Tumor Model

Experimental reagents: fetal bovine serum (SH30070.03) (FBS, Hyclone, Logan, UT, USA); penicillin (I9532) (Sigma, St. Louis, MO, USA); streptomycin (85886) (Sigma, St. Louis, MO, USA); recombinant human insulin (91077C) (Sigma, St. Louis, MO, USA); EMEM medium (30-2003) (ATCC, Rockville, MD, USA); trypsin (15090046) (Gibco, Grand Island, NY, USA); HBSS (H6648), DMSO (D8418), PEG400 (8074851000), PEG300 (8074841000), PBS (806552), Solutol HS-15 (42966) (Sigma, St. Louis, MO, USA); Matrigel Matrix (BD Bioscience, USA); estrogen pellets (0.36 mg estradiol, 60-day release) (SE-121) (Innovative Research of America, Florida, USA); PVDF membrane (0.45 μm) (Millipore, Schwalbach, Germany); StarSignal Western Protein Marker (10-200 kDa) (M227-01) (GenStar, Beijing, China); Ponceau red, Tween 20, acrylamide, sodium dodecyl sulfate, PMSF (Solon, OH, USA); protein blotting membrane regeneration solution (ZN1923, Biolab, Beijing, China); protein lysis buffer (RIPA), 1.5 mol/L Tris HCl (pH 6.8), 1.5 mol/L Tris HCl (pH 8.8) (Beyotime, Shanghai, China); ECL luminescent solution (Thermo Fisher Scientific, Pittsburgh, PA, USA); Anti-ERα antibody (21244-1-AP) purchased from Proteintech (Proteintech, Hubei, China); Goat Anti-Rabbit IgG H&L (HRP) (ab6721) purchased from Abcam (Abcam, Cambridge, UK).

Experimental animals: Female athymic nude mice, provided by Changzhou Cavens Laboratory Animal Co., Ltd., Animal Certificate No.: SCXK(Su)2016-0010, were kept in an environment of 22±2° C. and had free access to food and water.

Cell culture conditions: MCF-7 cells were subcultured in EMEM medium containing penicillin (final concentration of 100 U/mL), streptomycin (final concentration of 100 μg/mL), human recombinant insulin (final concentration of 0.01 mg/mL) and 10% FBS. When the cells were fused to 90%, the old medium was discarded, and the cells were washed twice with 2 ml PBS. After discarding PBS, 2 mL 0.25% trypsin-0.02% EDTA mixed digestion solution was added and observed under a microscope for about 30 s. When the cells became round, 2 ml complete medium was quickly added to terminate digestion, and the cells were gently blown and collected, and centrifuged at 800 rpm, 4° C. for 5 min. The supernatant was discarded, the cells were resuspended with complete medium, cultured in bottles, and the medium was changed every other day.

Blank group: 10% PEG300+25% of 20% Solutol+65% PBS, after the mouse MCF-7 transplanted tumor model was established and the average tumor volume reached about 200 mm³, gavage administration was performed daily, the gavage volume was 0.1 ml/10 g, and the administration was continued for 21 days (N=8);

LSZ102 (15 mg/kg) group: After the MCF-7 transplant tumor model was established and the average tumor volume reached about 200 mm³, LSZ102 was administered by gavage daily at a dose of 15 mg/kg and a gavage volume of 0.1 mL/10 g for 21 consecutive days (N=8); LSZ-102 is a selective estrogen receptor degrader developed by Novartis in the clinical research stage and is one of the earliest oral SERDs to enter clinical research;

Example 4 (5 mg/kg) group: After the mouse MCF-7 transplant tumor model was established and the average tumor volume reached about 200 mm³, Example 4 was administered by gavage every day, with a dosage of 5 mg/kg and a gavage volume of 0.1 mL/10 g, for 21 consecutive days (N=8).

The mice in each group were weighed and the tumor volume (width²×length×π/6) was measured every 3 days in the morning at a fixed time. The tumor inhibition efficacy of the compound was evaluated by tumor growth inhibition rate TGI (%). TGI (%)=[(1−(average tumor volume at the end of administration in a treatment group−average tumor volume at the beginning of administration in the treatment group)/(average tumor volume at the end of administration in the solvent control group−average tumor volume at the beginning of administration in the solvent control group)]×100%.

TABLE 9
Tumor volume of subcutaneous transplanted
tumor model of representative compounds
of the present invention (p.o., QD)

| Test | dose | mean tumour volume (mm³) | | |
|---|---|---|---|---|
| compounds | mg/kg | 0 day | 21 days | TGI(%) |
| blank group | / | 197 | 779 | / |
| LSZ102 | 15 | 200 | 345 | 75% |
| Example 4 | 5 | 202 | 332 | 78% |

The experimental results (Table 9 and FIG. 2) show that in the mouse subcutaneous transplant tumor MCF-7 model, compared with the blank group, the Example 4 compound of the present application has a significant inhibitory effect on tumor growth when orally administered once a day at 5 mg/kg, with a TGI of 78%, which is equivalent to the in vivo antitumor effect of LSZ102, a clinical phase I drug, at a threefold dose (15 mg/kg). It can be seen that the compound of the present invention exhibits a strong in vivo antitumor activity, which is about 3 times that of the control drug LSZ102.

The above results show that the compounds of the present invention can treat or prevent various diseases related to estrogen by antagonizing/degrading estrogen receptors, such as cancer (breast cancer, ovarian cancer, colon cancer, prostate cancer, endometrial cancer), osteoporosis, neurodegenerative disease, cardiovascular disease, lupus erythematosus, endometriosis and obesity.

Although the present invention has been described in detail above, it is understood by those skilled in the art that various modifications and changes may be made to the present invention without departing from the spirit and scope of the present invention. The scope of the present invention is not limited to the detailed description above, but as set forth in the appended claims.

1. A compound represented by general formula (I), or a racemate, enantiomer, diastereomer, or mixture thereof, or pharmaceutically acceptable salt thereof: 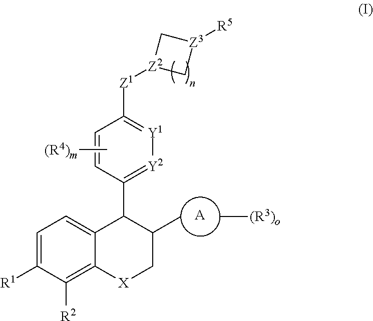
wherein,
R¹ is selected from OH, COOH, B(OH)₂, halogen, C₁-C₆ alkyl, halogenated C₁-C₆ alkyl or C₁-C₆ alkoxy; R² is selected from H, OH, COOH, halogen, cyano, C₁-C₆ alkyl, C₁-C₆ alkoxy, halogenated C₁-C₆ alkyl or hydroxy-substituted C₁-C₆ alkyl; or R¹, R² together with the connected benzene ring form a benzo 5-6 membered heteroaryl;
X is selected from S, S(O)₂ or O;
ring A is selected from C₃-C₆ cycloalkyl, 5-8 membered heterocyclyl, C₆-C₁₀ aryl or 5-8 membered heteroaryl;
each R³ is independently selected from hydrogen, halogen, cyano, C₁-C₆ alkylthio C₁-C₆ alkylCO—, C₁-C₆ alkylSO₂—, amino, —NH(C₁-C₆ alkyl), —N(C₁-C₆ alkyl)(C₁-C₆ alkyl), —SO₂NH₂, —C(O)NH₂, C₁-C₆ alkyl, C₁-C₆ alkoxy, halogenated C₁-C₆ alkyl, halogenated C₁-C₆ alkoxy or halogenated C₁-C₆ alkylthio;
o is 0, 1, 2, 3, or 4;
Y¹, Y² are independently selected from CR⁴ or N;
each R⁴ is independently selected from hydrogen, halogen, cyano, C₁-C₆ alkylthio, C₁-C₆ alkylCO—, C₁-C₆ alkylSO₂—, amino, —NH(C₁-C₆ alkyl), —N(C₁-C₆ alkyl)(C₁-C₆ alkyl), —SO₂NH₂, —C(O)NH₂, C₁-C₆ alkyl, C₁-C₆ alkoxy, halogenated C₁-C₆ alkyl, halogenated C₁-C₆ alkoxy or halogenated C₁-C₆ alkylthio;
m is 0, 1, 2, 3 or 4;
Z¹-Z² is selected from O—Z², NH—Z², S—Z², S(O)—Z², S(O)₂—Z², O—(C₁-C₆ alkylene)-Z², O-(halogenated C₁-C₆ alkylene)-Z², NH—(C₁-C₆ alkylene)-Z² or NH-(halogenated C₁-C₆ alkylene)-Z²;
Z², Z³ are independently selected from CH or N;
n is 1, 2 or 3;
R⁵ is C₁-C₆ alkyl, optionally substituted by one or more substituents selected from the group consisting of halogen, cyano, hydroxy, carboxyl, amino, methoxy or —SO₂CH₃;
the premise is that when X is S or S(O)₂, ring A is selected from C₃-C₆ cycloalkyl, 5-8 membered heterocyclyl, C₆-C₁₀ aryl or 5-8 membered heteroaryl; when X is O, ring A is selected from C₃-C₆ cycloalkyl, 5-8 membered heterocyclyl or 5-8 membered heteroaryl.

2. The compound represented by the general formula (I) of claim 1, or the racemate, enantiomer, diastereomer, or mixture thereof, or pharmaceutically acceptable salt thereof, wherein the compound represented by the general formula (I) is a compound represented by the general formula (Ia): 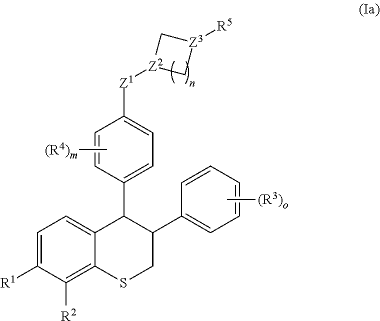
R¹, R², R³, o, R⁴, m, Z¹, Z², n, Z³, and R⁵ are defined as in claim 1.

3. The compound of the general formula (I) of claim 1, wherein the compound of the general formula (I) is a compound of the general formula (Ib): 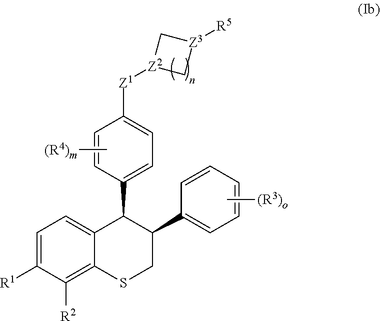
wherein, R¹, R², R³, o, R⁴, m, Z¹, Z², n, Z³, and R⁵ are defined as in claim 1.

4. The compound represented by the general formula (I) of claim 1, or the racemate, enantiomer, diastereomer, or mixture thereof, or pharmaceutically acceptable salt thereof, wherein
each R³ is independently selected from hydrogen, halogen, cyano, C₁-C₄ alkylthio, C₁-C₄ alkylCO—, C₁-C₄ alkylSO₂—, amino, —NH(C₁-C₄ alkyl), —N(C₁-C₄ alkyl)(C₁-C₄ alkyl), —SO₂NH₂, —C(O)NH₂, C₁-C₄ alkyl, C₁-C₄ alkoxy, halogenated C₁-C₄ alkyl, halogenated C₁-C₄ alkoxy or halogenated C₁-C₄ alkylthio;
o is 0, 1, 2, 3, or 4;
when X is S or S(O)₂, ring A is selected from C₃-C₆ cycloalkyl, 5-7 membered heterocyclyl, C₆-C₁₀ aryl or 5-7 membered heteroaryl; when X is O, ring A is selected from C₃-C₆ cycloalkyl, 5-7 membered heterocyclyl or 5-7 membered heteroaryl.

5. The compound represented by the general formula (I) of claim 1, or the racemate, enantiomer, diastereomer, or mixture thereof, or pharmaceutically acceptable salt thereof, wherein
Z¹-Z² is selected from O—Z², NH—Z², S—Z², S(O)—Z², S(O)₂—Z², O—(C₁-C₄ alkylene)-Z², O-(halogenated C₁-C₄ alkylene)-Z², NH—(C₁-C₄ alkylene)-Z² or NH-(halogenated C₁-C₄ alkylene)-Z²;
Z², Z³ are independently selected from CH or N;
n is 1, 2 or 3;
R⁵ is C₁-C₄ alkyl, optionally substituted by one or more substituents selected from the group consisting of fluorine, chlorine, bromine, cyano, hydroxyl, carboxyl, amino, methoxy or —SO₂CH₃.

6. The compound represented by the general formula (I) of claim 1, or the racemate, enantiomer, diastereomer, or mixture thereof, or pharmaceutically acceptable salt thereof, wherein
R¹ is selected from OH, COOH, B(OH)₂, R² is selected from H; or R¹, R² and the connected benzene ring form 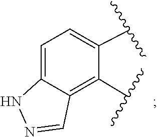 [SMILES: *c1ccc2[nH]ncc2c1*]
X is selected from S or S(O)₂; ring A is selected from C₃-C₆ cycloalkyl, 5-6 membered heterocyclyl, phenyl or 5-6 membered heteroaryl;
each R³ is independently selected from hydrogen, fluorine, chlorine, bromine, cyano, C₁-C₄ alkylthio, C₁-C₂ alkylCO—, C₁-C₂ alkylSO₂—, amino, —NH(C₁-C₂ alkyl), —N(C₁-C₂ alkyl)(C₁-C₂ alkyl), —SO₂NH₂, —C(O)NH₂, C₁-C₄ alkyl, C₁-C₄ alkoxy, halogenated C₁-C₄ alkyl, halogenated C₁-C₄ alkoxy or halogenated C₁-C₄ alkylthio;
o is 0, 1, 2, 3, or 4;
Y¹, Y² are independently selected from CR⁴ or N;
each R⁴ is independently selected from hydrogen, fluorine, chlorine, bromine, cyano, C₁-C₂ alkylthio, C₁-C₂ alkylCO—, C₁-C₂ alkylSO₂—, amino, —NH(C₁-C₂ alkyl), —N(C₁-C₂ alkyl)(C₁-C₂ alkyl), —SO₂NH₂, —C(O)NH₂, C₁-C₄ alkyl, C₁-C₄ alkoxy, halogenated C₁-C₄ alkyl, halogenated C₁-C₄ alkoxy or halogenated C₁-C₄ alkylthio;
m is 0, 1, 2 or 3;
Z¹-Z² is selected from O—Z², NH—Z², O—(C₁-C₄ alkylene)-Z², O-(halogenated C₁-C₄ alkylene)-Z², NH—(C₁-C₄ alkylene)-Z² or NH-(halogenated C₁-C₄ alkylene)-Z²;
Z², Z³ are independently selected from CH or N;
n is 1 or 2;
R⁵ is C₁-C₄ alkyl, optionally substituted by one or more substituents selected from the group consisting of fluorine, chlorine, bromine, cyano, hydroxyl or carboxyl.

7. The compound represented by the general formula (I) of claim 1, or the racemate, enantiomer, diastereomer, or mixture thereof, or pharmaceutically acceptable salt thereof, wherein the compound is selected from the following group:

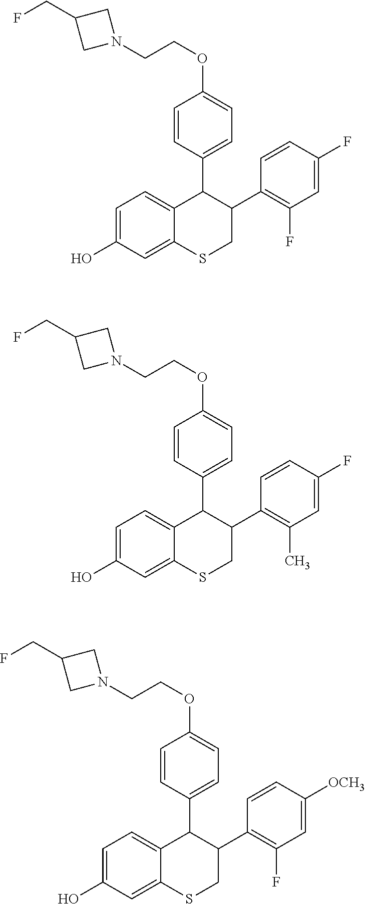

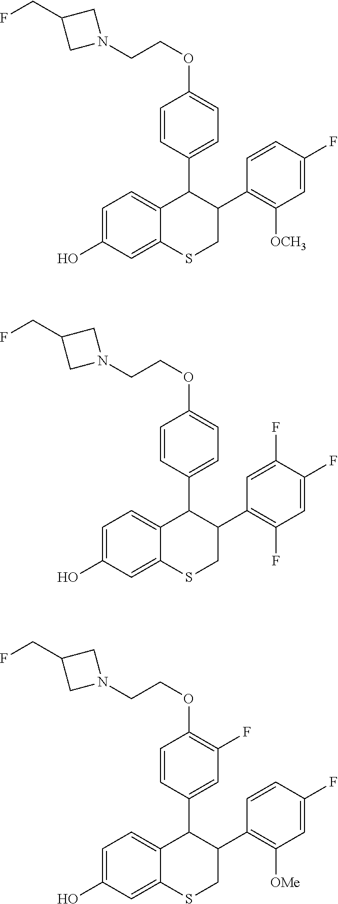

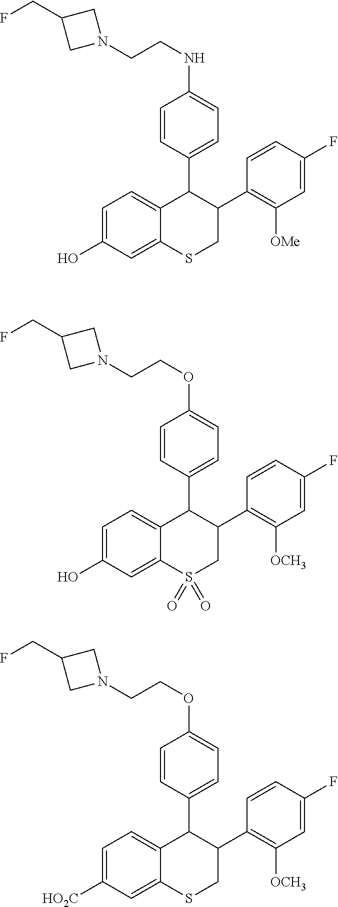

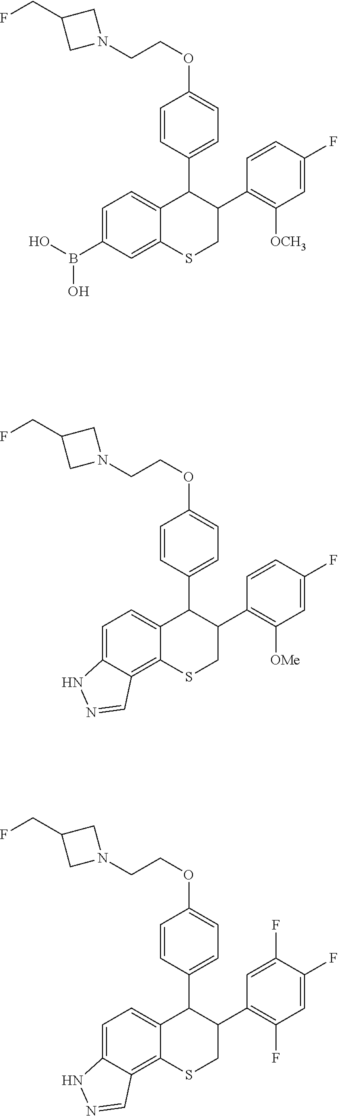

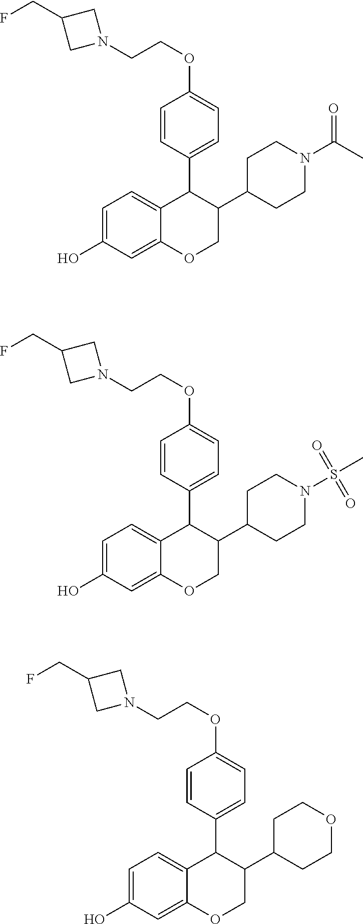

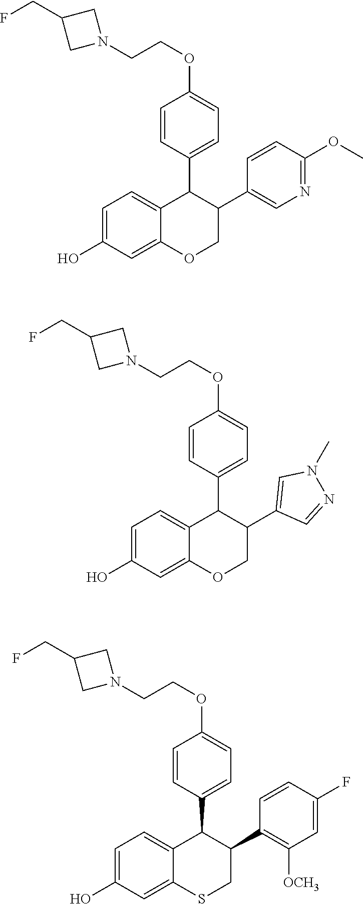

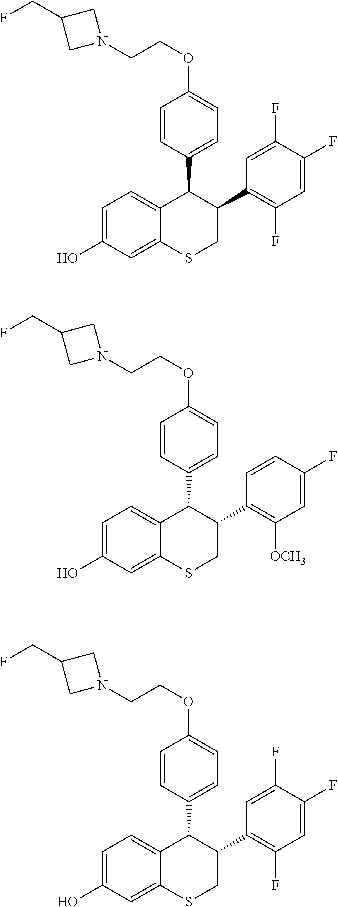

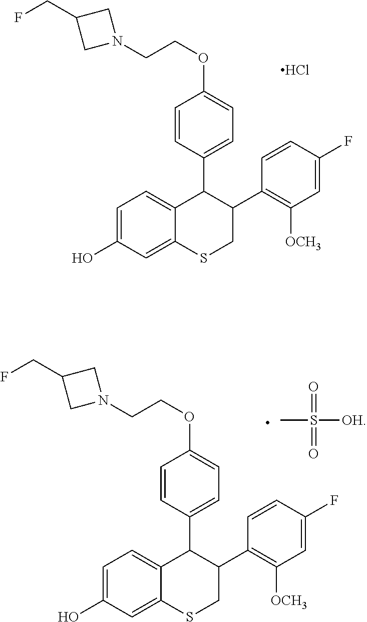

8. A method for preparing the compound represented by the general formula (I) of claim 1, wherein R¹ is OH, R² is hydrogen, Z¹ is OCH₂CH₂, and Z² is N, and the preparation method comprises the following steps: 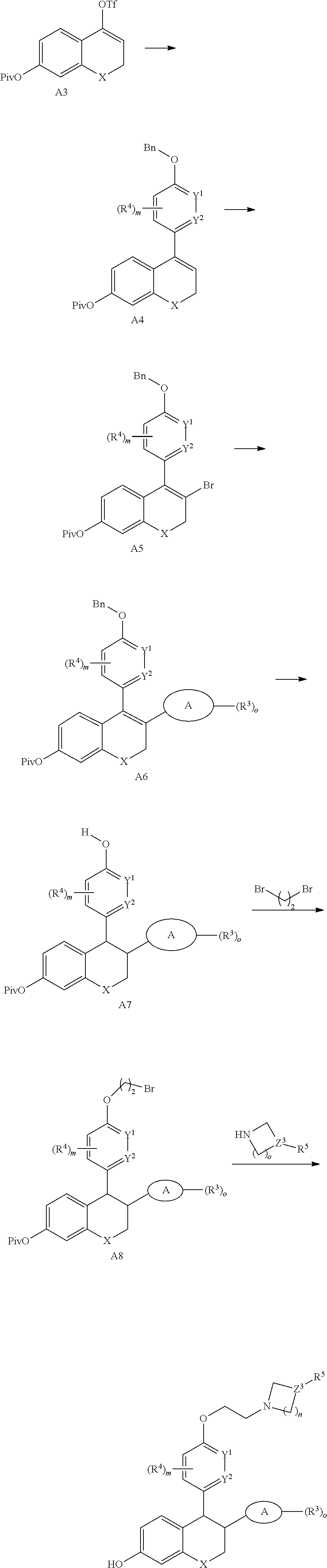
(i1) reacting A3 with an organic boron reagent through Suziki coupling reaction to obtain A4, wherein the organic boron reagent is selected from: boric acid 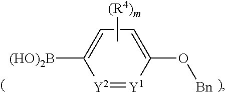
boric acid pinacol ester 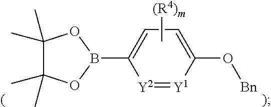
(i2) reacting A4 with a bromination reagent through bromination reaction to obtain an alkenyl bromide A5, wherein the bromination reagent is selected from: pyridinium tribromide, N-bromosuccinimide;
(i3) reacting A5 with an organic boron reagent through Suziki coupling reaction to obtain A6, wherein the organic boron reagent is selected from: boric acid 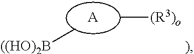
boric acid pinacol ester 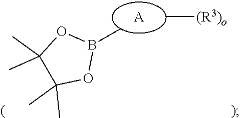
(i4) subjecting A6 to hydrogenation and hydrogenolysis reaction under the action of a palladium catalyst and hydrogen to obtain A7, wherein the palladium catalyst is selected from Pd/C and Pd(OH)₂/C;
(i5) subjecting A7 to nucleophilic substitution reaction to obtain A8;
(i6) subjecting A8 to nucleophilic substitution reaction under alkaline condition and hydrolysis to obtain a compound of formula (I), wherein a base used is selected from triethylamine, N,N-diisopropylethylamine, pyridine, carbonate, NaH, sodium hydroxide, potassium hydroxide, lithium hydroxide, sodium methoxide, and sodium ethoxide;
X, R⁴, Y², Y¹, ring A, R³, o, m, Z³, and R⁵ are as defined above;
or the preparation method comprises the following steps: 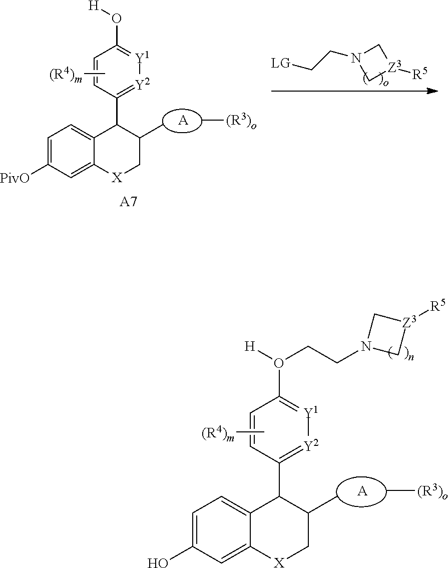
wherein, reacting A7 with 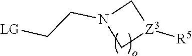
to obtain a compound of formula (I),
LG is a leaving group selected from Br, Cl, OTf, OTs or OMs;
X, R⁴, Y², Y¹, ring A, R³, o, m, Z³, and R⁵ are as defined above;
or R¹ is —COOH, R² is hydrogen, and the preparation method comprises the following steps: 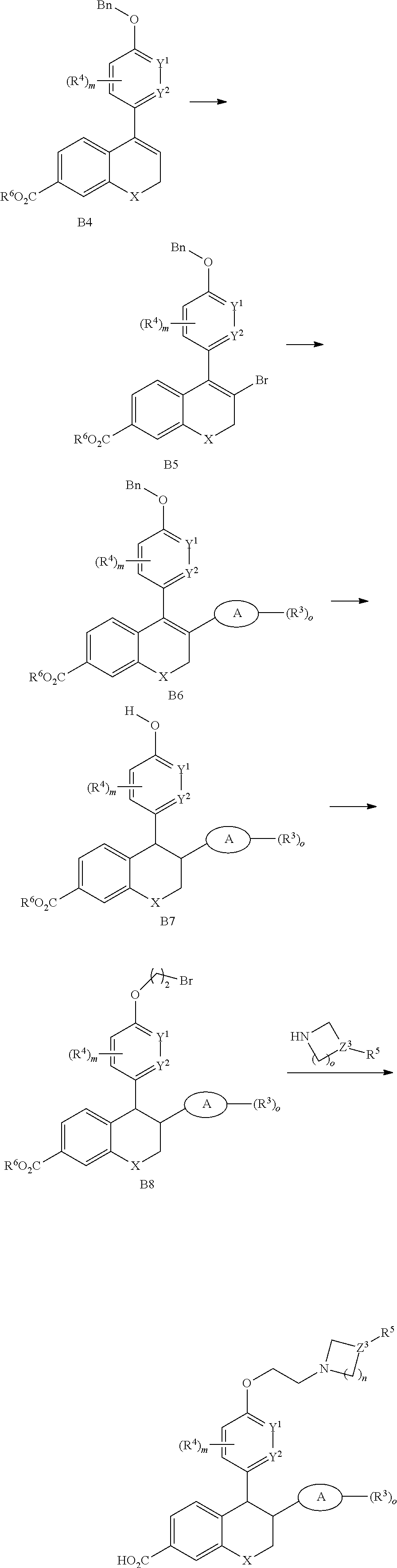
(ii1) reacting B4 with a bromination reagent through bromination reaction to obtain an alkenyl bromide B5, wherein the bromination reagent is selected from pyridinium tribromide, N-bromosuccinimide;
(ii2) reacting B5 with an organic boron reagent through Suziki coupling reaction to obtain B6, wherein the organic boron reagent is selected from boric acid 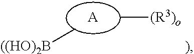
boric acid pinacol ester 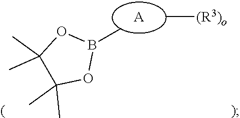
(ii3) subjecting B6 to hydrogenation and hydrogenolysis reaction under the action of a palladium catalyst and hydrogen to obtain B7, wherein the palladium catalyst is selected from Pd/C and Pd(OH)₂/C;
(ii4) subjecting B7 to nucleophilic substitution reaction to obtain B8;
(ii5) subjecting B8 to nucleophilic substitution reaction under alkaline condition and hydrolysis to obtain a compound of formula (I), wherein a base used is selected from triethylamine, N,N-diisopropylethylamine, pyridine, carbonate, NaH, sodium hydroxide, potassium hydroxide, lithium hydroxide, sodium methoxide, and sodium ethoxide;
X, R⁴, Y², Y¹, ring A, R³, o, m, Z³, and R⁵ are as defined above, and R⁶ is C₁-C₆ alkyl;
or when R¹, R² and the connected benzene ring form 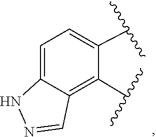 [SMILES: *c1ccc2[nH]ncc2c1*]
the preparation method comprises the following steps: 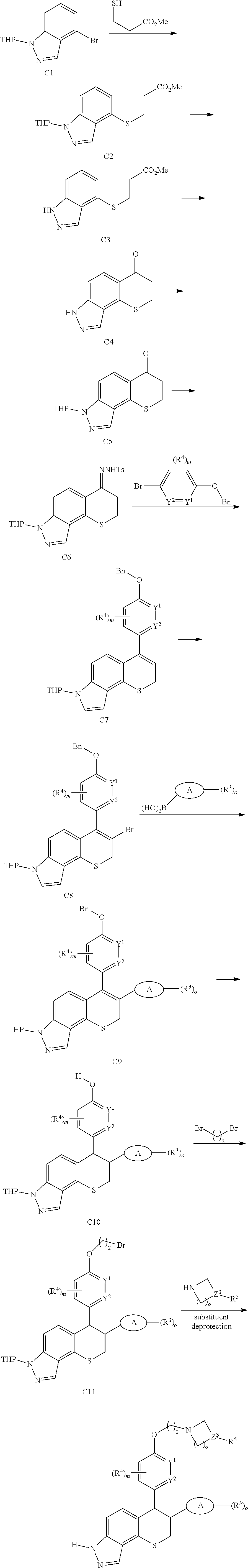
(iii1) reacting C1 and methyl 3-mercaptopropionate through C—S coupling under the action of a palladium catalyst to obtain C2, wherein the palladium catalyst is selected from [1,1′-bis(diphenylphosphino)ferrocene]palladium dichloride, tetrakis(triphenylphosphine) palladium, tris(dibenzylideneacetone)dipalladium, bis(dibenzylideneacetone)palladium, bistriphenylphosphinepalladium dichloride, di(tri-tert-butylphosphine)palladium, bis(tricyclohexylphosphine)palladium, and palladium acetate;
(iii2) hydrolyzing C2 under an acidic condition to obtain C3, wherein an acid is selected from sulfuric acid, hydrochloric acid, phosphoric acid, methanesulfonic acid, trifluoroacetic acid, acetic acid, and trifluoromethanesulfonic acid;
(iii3) subjecting C3 to Friedel-Crafts reaction under the action of an acid to obtain C4, wherein the acid is selected from the group consisting of trifluoromethanesulfonic acid, trifluoroacetic acid, Eaton's reagent, polyphosphoric acid, sulfuric acid, and hydrochloric acid;
(iii4) reacting C4 with dihydropyran in the presence of an acid to obtain C5, wherein the acid includes p-toluenesulfonic acid, methanesulfonic acid, and p-toluenesulfonic acid pyridinium salt;
(iii5) reacting C5 with p-toluenesulfonyl hydrazide to obtain hydrazone C6;
(iii6) reacting C6 with an aromatic bromide in the presence of a palladium catalyst to obtain C7, wherein the palladium catalyst is selected from [1,1′-bis(diphenylphosphino)ferrocene]palladium dichloride, tetrakis(triphenylphosphine) palladium, tris(dibenzylideneacetone)dipalladium, bis(dibenzylideneacetone)palladium, bistriphenylphosphinepalladium dichloride, di(tri-tert-butylphosphine)palladium, bis(tricyclohexylphosphine)palladium, and palladium acetate;
(iii7) reacting C7 with a bromination reagent through bromination reaction to obtain an alkenyl bromide C8, wherein the bromination reagent is selected from: pyridinium tribromide, N-bromosuccinimide;
(iii8) reacting C8 with an organic boron reagent through Suziki coupling reaction to obtain C9, wherein the organic boron reagent is selected from: boric acid 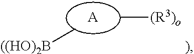
boric acid pinacol ester 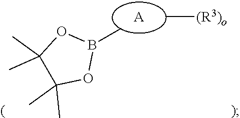
(iii9) subjecting C9 to hydrogenation and hydrogenolysis reaction under the action of a palladium catalyst and hydrogen to obtain C10, wherein the palladium catalyst is selected from Pd/C and Pd(OH)₂/C;
(iii10) subjecting C10 to nucleophilic substitution reaction to obtain C11;
(iii11) subjecting C11 to nucleophilic substitution reaction and hydrolysis under an acidic condition to obtain a compound of formula (I), wherein the acid is selected from trifluoromethanesulfonic acid, trifluoroacetic acid, sulfuric acid, hydrochloric acid, p-toluenesulfonic acid, and a solution of hydrogen chloride in an organic solvent, the organic solvent is methanol, ethanol, isopropanol, ethyl acetate, diethyl ether or 1,4-dioxane;
X, R⁴, Y², Y¹, ring A, R³, o, m, Z³, and R⁵ are defined as above.

9. A pharmaceutical composition comprising:
the compound represented by the general formula (I) of claim 1, or a racemate, enantiomer, diastereomer, or mixture thereof, or a pharmaceutically acceptable salt thereof; and
a pharmaceutically acceptable carrier.

10. A method for treating, preventing or diagnosing an estrogen receptor-related disease comprising administering the compound represented by the general formula (I) of claim 1, or a racemate, enantiomer, diastereoisomer, or mixture thereof, or pharmaceutically acceptable salt thereof to a subject in need thereof.

11. The method of claim 10, wherein the disease is selected from the group consisting of breast cancer, endometrial cancer, cervical cancer, skin cancer, prostate cancer, ovarian cancer, fallopian tube tumor, lung cancer, leukemia, osteoporosis, neurodegenerative disease, cardiovascular disease, lupus erythematosus, endometriosis and obesity.
